SEQUENCE LISTING (202 sequences)

SEQ ID NO: 1 - protein, 27 aa, synthetic construct
LHSGQNHLKEMAISVLEARACAAAGQS

SEQ ID NO: 2 - protein, 25 aa, synthetic construct
LWPKVTYHSPSYVYHQFERKTKYIR

SEQ ID NO: 3 - protein, 27 aa, synthetic construct
DTLSAMSNPRAMQVLLQIQQGLQTLAT

SEQ ID NO: 4 - protein, 27 aa, synthetic construct
YRGANLHLEETLAGFWARLLERLFKQL

SEQ ID NO: 5 - protein, 27 aa, synthetic construct
VILPQAPSGPSYATYLQPAQAQMLTPP

SEQ ID NO: 6 - protein, 11 aa, synthetic construct
NHLKEMAISVL

SEQ ID NO: 7 - protein, 9 aa, synthetic construct
SPSYVYHQF

SEQ ID NO: 8 - protein, 9 aa, synthetic construct
NPRAMQVLL

SEQ ID NO: 9 - protein, 10 aa, synthetic construct
GFWARLLERL

SEQ ID NO: 10 - protein, 9 aa, synthetic construct
SGPSYATYL

SEQ ID NO: 11 - protein, 15 aa, synthetic construct
NHLKEMAISVLEARA

SEQ ID NO: 12 - protein, 15 aa, synthetic construct
SYVYHQFERKTKYIR

SEQ ID NO: 13 - protein, 15 aa, synthetic construct
MSNPRAMQVLLQIQQ

SEQ ID NO: 14 - protein, 15 aa, synthetic construct
LEETLAGFWARLLER

SEQ ID NO: 15 - protein, 15 aa, synthetic construct
PSYATYLQPAQAQML

SEQ ID NO: 16 - protein, 21 aa, synthetic construct
MSYAEKSDEITKDEWMEKLNN

SEQ ID NO: 17 - protein, 30 aa, synthetic construct
NLGLDLRTAAYVNAIEKIFKVYNEAGVTFT

SEQ ID NO: 18 - protein, 21 aa, synthetic construct
KKLIYAAKLNTSLKALEGERN

SEQ ID NO: 19 - protein, 30 aa, synthetic construct
IAHMILGYRYWTGIGVLQSCESALTHYRLV

SEQ ID NO: 20 - protein, 30 aa, synthetic construct
EPTETFYYIDDPNGLEISIIAKFLYGKNVD

SEQ ID NO: 21 - protein, 30 aa, synthetic construct
RSVNSVLLFTILNPIYSRTTDVLYTICNPC

SEQ ID NO: 22 - protein, 29 aa, synthetic construct
EDVVMMSTPVVKAALKMGFSRSLVRQTVQ

SEQ ID NO: 23 - protein, 29 aa, synthetic construct
KRASSENERLQYKTPLPSYNSALTQPGVA

SEQ ID NO: 24 - protein, 29 aa, synthetic construct
LHSGQNHLKEMAISVLEARACAAAGQSGL

SEQ ID NO: 25 - protein, 29 aa, synthetic construct
VTLGKSFLQFSAMKMVHSPPGHHSVTGRP

SEQ ID NO: 26 - protein, 29 aa, synthetic construct
SLYYTTVRALTRHNTMLKAMFSGRMEVLT

SEQ ID NO: 27 - protein, 29 aa, synthetic construct
IEPVGLQPIRFVKASAVERGGPKKCALTG

SEQ ID NO: 28 - protein, 30 aa, synthetic construct
MSGFQCDLTANNSIALKSSKLLYIYGSLDS

SEQ ID NO: 29 - protein, 30 aa, synthetic construct
PPYSLEHAKITSETNTQLEHAKITQTELMR

SEQ ID NO: 30 - protein, 29 aa, synthetic construct
EVEVVPSNMWGDQGLLGASVRFCSFRRAS

SEQ ID NO: 31 - protein, 27 aa, synthetic construct
GPGTTYNFPQSPSGNEMTNNQPKTAKA

SEQ ID NO: 32 - protein, 29 aa, synthetic construct
WKEVRHDNKVIWLVSWTENIQGSIKYIML

SEQ ID NO: 33 - protein, 29 aa, synthetic construct
PKGEPGIPGDQDLQGPPGIPGIVGPSGPI

SEQ ID NO: 34 - protein, 30 aa, synthetic construct
AIPSPAVQDGCTHYTRDPRLRACSEQTACR

SEQ ID NO: 35 - protein, 9 aa, synthetic construct
SYAEKSDEI

SEQ ID NO: 36 - protein, 9 aa, synthetic construct
AYVNAIEKI

SEQ ID NO: 37 - protein, 10 aa, synthetic construct
IYAAKLNTSL

SEQ ID NO: 38 - protein, 9 aa, synthetic construct
RYWTGIGVL

SEQ ID NO: 39 - protein, 11 aa, synthetic construct
YYIDDPNGLEI

SEQ ID NO: 40 - protein, 9 aa, synthetic construct
IYSRTTDVL

SEQ ID NO: 41 - protein, 9 aa, synthetic construct
KMGFSRSLV

SEQ ID NO: 42 - protein, 10 aa, synthetic construct
TPLPSYNSAL

SEQ ID NO: 43 - protein, 10 aa, synthetic construct
NHLKEMAISV

SEQ ID NO: 44 - protein, 11 aa, synthetic construct
SFLQFSAMKMV

SEQ ID NO: 45 - protein, 9 aa, synthetic construct
RHNTMLKAM

SEQ ID NO: 46 - protein, 10 aa, synthetic construct
RGGPKKCALT

SEQ ID NO: 47 - protein, 9 aa, synthetic construct
KLLYIYGSL

SEQ ID NO: 48 - protein, 9 aa, synthetic construct
PYSLEHAKI

SEQ ID NO: 49 - protein, 11 aa, synthetic construct
VPSNMWGDQGL

SEQ ID NO: 50 - protein, 11 aa, synthetic construct
FPQSPSGNEMT

SEQ ID NO: 51 - protein, 11 aa, synthetic construct
VIWLVSWTENI

SEQ ID NO: 52 - protein, 11 aa, synthetic construct
EPGIPGDQDLQ

SEQ ID NO: 53 - protein, 9 aa, synthetic construct
LRACSEQTA

SEQ ID NO: 54 - protein, 16 aa, synthetic construct
EKSDEITKDEWMEKLN

SEQ ID NO: 55 - protein, 15 aa, synthetic construct
IEKIFKVYNEAGVTF

SEQ ID NO: 56 - protein, 17 aa, synthetic construct
KKLIYAAKLNTSLKALE

SEQ ID NO: 57 - protein, 15 aa, synthetic construct
YWTGIGVLQSCESAL

SEQ ID NO: 58 - protein, 15 aa, synthetic construct
IDDPNGLEISIIAKF

SEQ ID NO: 59 - protein, 15 aa, synthetic construct
LFTILNPIYSRTTDV

SEQ ID NO: 60 - protein, 19 aa, synthetic construct
VMMSTPVVKAALKMGFSRS

SEQ ID NO: 61 - protein, 19 aa, synthetic construct
NERLQYKTPLPSYNSALTQ

SEQ ID NO: 62 - protein, 15 aa, synthetic construct
NHLKEMAISVLEARA

SEQ ID NO: 63 - protein, 19 aa, synthetic construct
GKSFLQFSAMKMVHSPPGH

SEQ ID NO: 64 - protein, 15 aa, synthetic construct
SLYYTTVRALTRHNT

SEQ ID NO: 65 - protein, 19 aa, synthetic construct
EPVGLQPIRFVKASAVERG

SEQ ID NO: 66 - protein, 19 aa, synthetic construct
CDLTANNSIALKSSKLLYI

SEQ ID NO: 67 - protein, 15 aa, synthetic construct
NTQLEHAKITQTELM

SEQ ID NO: 68 - protein, 17 aa, synthetic construct
MWGDQGLLGASVRFCSF

SEQ ID NO: 69 - protein, 16 aa, synthetic construct
TYNFPQSPSGNEMTNN

SEQ ID NO: 70 - protein, 19 aa, synthetic construct
DNKVIWLVSWTENIQGSIK

SEQ ID NO: 71 - protein, 19 aa, synthetic construct
QDLQGPPGIPGIVGPSGPI

SEQ ID NO: 72 - protein, 15 aa, synthetic construct
RDPRLRACSEQTACR

SEQ ID NO: 73 - protein, 30 aa, synthetic construct
ESKNILAVSFAPLVQLSKNDNGTPDSVGLP

SEQ ID NO: 74 - protein, 30 aa, synthetic construct
WQQVTPMAPAAATLTFEGMAGSQRLGDWGK

SEQ ID NO: 75 - protein, 30 aa, synthetic construct
LPGGGAGHLDQNVFLRVRALCYVISGQGQR

SEQ ID NO: 76 - protein, 30 aa, synthetic construct
PQDMHKCLARSHVARNRFVQILQREHYVMQ

SEQ ID NO: 77 - protein, 30 aa, synthetic construct
QGFNVFGMPSTSGASNTTPHLGFHLGSKGT

SEQ ID NO: 78 - protein, 30 aa, synthetic construct
AKLWACNFCYQRNQLPPTYAGISELNQPAE

SEQ ID NO: 79 - protein, 27 aa, synthetic construct
DTLMYVYEMLGAGLLSNLYDKLGRLLT

SEQ ID NO: 80 - protein, 27 aa, synthetic construct
GANQLVVLSVYDMYWMNEYTSSIGIGV

SEQ ID NO: 81 - protein, 27 aa, synthetic construct
TLLSPEELKAASRGNGEYVWYYEGRNG

SEQ ID NO: 82 - protein, 27 aa, synthetic construct
KSEAQCKNFYFNYKRRPNLDNLLQQHK

SEQ ID NO: 83 - protein, 27 aa, synthetic construct
RLVPVMNDCYILLFNQDTNMLHSIQDR

SEQ ID NO: 84 - protein, 27 aa, synthetic construct
TYSFSQVPRYLSGFWSEFSTRSENFLK

SEQ ID NO: 85 - protein, 30 aa, synthetic construct
IDLKAPVIVIPQSSLPTNAVVVDLGLIRVH

SEQ ID NO: 86 - protein, 30 aa, synthetic construct
MTTSTSPAAMPLRRLRRLSWGSTAVQLFIL

SEQ ID NO: 87 - protein, 30 aa, synthetic construct
NHSQSLALVIEDKMKRYKISGNNPFFGKLQ

SEQ ID NO: 88 - protein, 30 aa, synthetic construct
RNVDSTCEDREDKFNFSVMSYNILSQDLLE

SEQ ID NO: 89 - protein, 30 aa, synthetic construct
TAKHGLNRTDYYIMNGPQLSFLDAYRNYAQ

SEQ ID NO: 90 - protein, 30 aa, synthetic construct
LVVQLLQFQEEVFAKHVSNAPLTKLPIKCF

SEQ ID NO: 91 - protein, 30 aa, synthetic construct
SYLPSQVTEMFNQGRAFAAVRLPFCGHKNI

SEQ ID NO: 92 - protein, 30 aa, synthetic construct
QTPQSPHPNAEKGPFEFVSSPAGNTSVMLV

SEQ ID NO: 93 - protein, 30 aa, synthetic construct
EGGGGSGTYRVGNAQPSLADCLDAGDLAQR

SEQ ID NO: 94 - protein, 30 aa, synthetic construct
NRQDPFTVHMAARIIATLAAWGKELMEGSD

SEQ ID NO: 95 - protein, 30 aa, synthetic construct
INRQKSKYNLPLTKIISAKRNESDFWQDSA

SEQ ID NO: 96 - protein, 30 aa, synthetic construct
YDPAADRWEPRAPMRAPRVLHAMLGAAGRI

SEQ ID NO: 97 - protein, 30 aa, synthetic construct
AGDGPAGLTTSEGAMARATVSEQDSLNNNE

SEQ ID NO: 98 - protein, 30 aa, synthetic construct
KHIKAFDRTFANNPGPMVVFATPGMLHAGQ

SEQ ID NO: 99 - protein, 30 aa, synthetic construct
YDQIRYISQTQGLPAGYLLSAGTKTTRFFN

SEQ ID NO: 100 - protein, 30 aa, synthetic construct
GPPVNNSGSISPSAHPMPSEAKMRLKATLH

SEQ ID NO: 101 - protein, 30 aa, synthetic construct
AQEQAFLAQLKGSKALATSQPLTDSEPSQK

SEQ ID NO: 102 - protein, 30 aa, synthetic construct
AFLSHILSLVSQSNPKAAQTQIDAVCCRRC

SEQ ID NO: 103 - protein, 27 aa, synthetic construct
CVDCLDRTNTAQVMVGKCALAYQLYSL

SEQ ID NO: 104 - protein, 28 aa, synthetic construct
LFSMAFPDNQRPFLKAESKCPLNEEDTL

SEQ ID NO: 105 - protein, 27 aa, synthetic construct
PSLVRFGGKAREYSPTARIRSWMGYEL

SEQ ID NO: 106 - protein, 27 aa, synthetic construct
LVKLTFNAFTLEEPSLPGKCTFDYVQI

SEQ ID NO: 107 - protein, 27 aa, synthetic construct
PQPSKRVGSLTPPKSPRRSDSIKFQHR

SEQ ID NO: 108 - protein, 27 aa, synthetic construct
NDSILFVNEVDVQEVTHSAAVEALKEA

SEQ ID NO: 109 - protein, 30 aa, synthetic construct
LQKRLRSNWKIQSLKAEITSEKLIGVKLWI

SEQ ID NO: 110 - protein, 30 aa, synthetic construct
KPLEAVLRYLETHPRLPKPDPVKSSSSVLS

SEQ ID NO: 111 - protein, 30 aa, synthetic construct
NRQAQIEVVPSASALINKALKEPPRDRKKQ

SEQ ID NO: 112 - protein, 30 aa, synthetic construct
WILGDVFIGSYYTVFDRDNNRVSFANAVVL

SEQ ID NO: 113 - protein, 30 aa, synthetic construct
QAQESYEKAMDKAKREHERSNASPAIFPEY

SEQ ID NO: 114 - protein, 30 aa, synthetic construct
EELAWHTNLSRKILRMSPLLAKFHQFLVSE

SEQ ID NO: 115 - protein, 30 aa, synthetic construct; identical to SEQ ID NO: 73

SEQ ID NO: 116 - protein, 9 aa, synthetic construct
VSFAPLVQL

SEQ ID NO: 117 - protein, 8 aa, synthetic construct
ATLTFEGM

SEQ ID NO: 118 - protein, 8 aa, synthetic construct
VFLRVRAL

SEQ ID NO: 119 - protein, 9 aa, synthetic construct
VARNRFVQI

SEQ ID NO: 120 - protein, 10 aa, synthetic construct
SGASNTTPHL

SEQ ID NO: 121 - protein, 10 aa, synthetic construct
CNFCYQRNQL

SEQ ID NO: 122 - protein, 10 aa, synthetic construct
YVYEMLGAGL

SEQ ID NO: 123 - protein, 8 aa, synthetic construct
SVYDMYWM

SEQ ID NO: 124 - protein, 11 aa, synthetic construct
KAASRGNGEYV

SEQ ID NO: 125 - protein, 9 aa, synthetic construct
FNYKRRPNL

SEQ ID NO: 126 - protein, 10 aa, synthetic construct
ILLFNQDTNM

SEQ ID NO: 127 - protein, 9 aa, synthetic construct
QVPRYLSGF

SEQ ID NO: 128 - protein, 10 aa, synthetic construct
SSLPTNAVVV

SEQ ID NO: 129 - protein, 8 aa, synthetic construct
TSPAAMPL

SEQ ID NO: 130 - protein, 11 aa, synthetic construct
ISGNNPFFGKL

SEQ ID NO: 131 - protein, 11 aa, synthetic construct
FNFSVMSYNIL

SEQ ID NO: 132 - protein, 9 aa, synthetic construct
YYIMNGPQL

SEQ ID NO: 133 - protein, 10 aa, synthetic construct
FAKHVSNAPL

SEQ ID NO: 134 - protein, 8 aa, synthetic construct
QGRAFAAV

SEQ ID NO: 135 - protein, 9 aa, synthetic construct
KGPFEFVSS

SEQ ID NO: 136 - protein, 9 aa, synthetic construct
AQPSLADCL

SEQ ID NO: 137 - protein, 9 aa, synthetic construct
MAARIIATL

SEQ ID NO: 138 - protein, 9 aa, synthetic construct
YNLPLTKII

SEQ ID NO: 139 - protein, 9 aa, synthetic construct
RAPMRAPRV

SEQ ID NO: 140 - protein, 8 aa, synthetic construct
GAMARATV

SEQ ID NO: 141 - protein, 10 aa, synthetic construct
RTFANNPGPM

SEQ ID NO: 142 - protein, 9 aa, synthetic construct
AGYLLSAGT

SEQ ID NO: 143 - protein, 8 aa, synthetic construct
ISPSAHPM

SEQ ID NO: 144 - protein, 9 aa, synthetic construct
KALATSQPL

SEQ ID NO: 145 - protein, 9 aa, synthetic construct
AAQTQIDAV

SEQ ID NO: 146 - protein, 9 aa, synthetic construct
VMVGKCALA

SEQ ID NO: 147 - protein, 10 aa, synthetic construct
KCPLNEEDTL

SEQ ID NO: 148 - protein, 8 aa, synthetic construct
KAREYSPT

SEQ ID NO: 149 - protein, 10 aa, synthetic construct
NAFTLEEPSL

SEQ ID NO: 150 - protein, 9 aa, synthetic construct
KSPRRSDSI

SEQ ID NO: 151 - protein, 10 aa, synthetic construct
VQEVTHSAAV

SEQ ID NO: 152 - protein, 9 aa, synthetic construct
KIQSLKAEI

SEQ ID NO: 153 - protein, 9 aa, synthetic construct
RYLETHPRL

SEQ ID NO: 154 - protein, 8 aa, synthetic construct
SALINKAL

SEQ ID NO: 155 - protein, 9 aa, synthetic construct
VSFANAVVL

SEQ ID NO: 156 - protein, 9 aa, synthetic construct
KAMDKAKRE

SEQ ID NO: 157 - protein, 8 aa, synthetic construct
KILRMSPL

SEQ ID NO: 158 - protein, 9 aa, synthetic construct
VSFAPLVQL

SEQ ID NO: 159 - protein, 16 aa, synthetic construct
KNILAVSFAPLVQLSK

SEQ ID NO: 160 - protein, 16 aa, synthetic construct
AATLTFEGMAGSQRLG

SEQ ID NO: 161 - protein, 19 aa, synthetic construct
LDQNVFLRVRALCYVISGQ

SEQ ID NO: 162 - protein, 19 aa, synthetic construct
QDMHKCLARSHVARNRFVQ

SEQ ID NO: 163 - protein, 16 aa, synthetic construct
GFNVFGMPSTSGASNT

SEQ ID NO: 164 - protein, 16 aa, synthetic construct
RNQLPPTYAGISELNQ

SEQ ID NO: 165 - protein, 19 aa, synthetic construct
VYEMLGAGLLSNLYDKLGR

SEQ ID NO: 166 - protein, 19 aa, synthetic construct
SVYDMYWMNEYTSSIGIGV

SEQ ID NO: 167 - protein, 19 aa, synthetic construct
TLLSPEELKAASRGNGEYV

SEQ ID NO: 168 - protein, 16 aa, synthetic construct
NFYFNYKRRPNLDNLL

SEQ ID NO: 169 - protein, 16 aa, synthetic construct
YILLFNQDTNMLHSIQ

SEQ ID NO: 170 - protein, 16 aa, synthetic construct
YLSGFWSEFSTRSENF

SEQ ID NO: 171 - protein, 16 aa, synthetic construct
VIVIPQSSLPTNAVVV

SEQ ID NO: 172 - protein, 16 aa, synthetic construct
MTTSTSPAAMPLRRLR

SEQ ID NO: 173 - protein, 16 aa, synthetic construct
MKRYKISGNNPFFGKL

SEQ ID NO: 174 - protein, 16 aa, synthetic construct
REDKFNFSVMSYNILS

SEQ ID NO: 175 - protein, 16 aa, synthetic construct
TDYYIMNGPQLSFLDA

SEQ ID NO: 176 - protein, 16 aa, synthetic construct
EEVFAKHVSNAPLTKL

SEQ ID NO: 177 - protein, 19 aa, synthetic construct
SQVTEMFNQGRAFAAVRLP

SEQ ID NO: 178 - protein, 19 aa, synthetic construct
AEKGPFEFVSSPAGNTSVM

SEQ ID NO: 179 - protein, 19 aa, synthetic construct
GGSGTYRVGNAQPSLADCL

SEQ ID NO: 180 - protein, 19 aa, synthetic construct
RQDPFTVHMAARIIATLAA

SEQ ID NO: 181 - protein, 15 aa, synthetic construct
KSKYNLPLTKIISAK

SEQ ID NO: 182 - protein, 16 aa, synthetic construct
EPRAPMRAPRVLHAML

SEQ ID NO: 183 - protein, 19 aa, synthetic construct
DGPAGLTTSEGAMARATVS

SEQ ID NO: 184 - protein, 19 aa, synthetic construct
TFANNPGPMVVFATPGMLH

SEQ ID NO: 185 - protein, 19 aa, synthetic construct
TQGLPAGYLLSAGTKTTRF

SEQ ID NO: 186 - protein, 19 aa, synthetic construct
VNNSGSISPSAHPMPSEAK

SEQ ID NO: 187 - protein, 16 aa, synthetic construct
FLAQLKGSKALATSQP

SEQ ID NO: 188 - protein, 19 aa, synthetic construct
SHILSLVSQSNPKAAQTQI

SEQ ID NO: 189 - protein, 19 aa, synthetic construct
DCLDRTNTAQVMVGKCALA

SEQ ID NO: 190 - protein, 19 aa, synthetic construct
LFSMAFPDNQRPFLKAESK

SEQ ID NO: 191 - protein, 19 aa, synthetic construct
LVRFGGKAREYSPTARIRS

SEQ ID NO: 192 - protein, 16 aa, synthetic construct
VKLTFNAFTLEEPSLP

SEQ ID NO: 193 - protein, 16 aa, synthetic construct
PSKRVGSLTPPKSPRR

SEQ ID NO: 194 - protein, 16 aa, synthetic construct
VDVQEVTHSAAVEALK

SEQ ID NO: 195 - protein, 19 aa, synthetic construct
RLRSNWKIQSLKAEITSEK

SEQ ID NO: 196 - protein, 19 aa, synthetic construct
AVLRYLETHPRLPKPDPVK

SEQ ID NO: 197 - protein, 16 aa, synthetic construct
QIEVVPSASALINKAL

SEQ ID NO: 198 - protein, 16 aa, synthetic construct
FDRDNNRVSFANAVVL

SEQ ID NO: 199 - protein, 16 aa, synthetic construct
KAKREHERSNASPAIF

SEQ ID NO: 200 - protein, 16 aa, synthetic construct
LSRKILRMSPLLAKFH

SEQ ID NO: 201 - protein, 16 aa, synthetic construct
KNILAVSFAPLVQLSK

SEQ ID NO: 202 - protein, 5 aa, synthetic construct
GGGGS

(19) United States
(12) Patent Application Publication
(10) Pub. No.: US 2026/0166133 A1
(43) Pub. Date: Jun. 18, 2026

(54) Engineered Microbes Comprising Tumor Neoantigen Vectors for Cancer Immunotherapy

(71) Applicant: The Trustees of Columbia University in the City of New York, New York, NY (US)

(72) Inventors: Andrew REDENTI, New York, NY (US); Tal DANINO, Brooklyn, NY (US); Jongwon IM, New York, NY (US); Nicholas ARPAIA, New York, NY (US)

(73) Assignee: The Trustees of Columbia University in the City of New York, New York, NY (US)

(21) Appl. No.: 19/357,671

(22) Filed: Oct. 14, 2025

Related U.S. Application Data
(63) Continuation of application No. PCT/US2024/024327, filed on Apr. 12, 2024.
(60) Provisional application No. 63/459,400, filed on Apr. 14, 2023.
(60) Provisional application No. 63/541,624, filed on Sep. 29, 2023.
(60) Provisional application No. 63/552,806, filed on Feb. 13, 2024.

(51) Int. Cl. A61K 39/00; A61P 37/04; C12N 15/70; C12Q 1/6886; G16B 15/30

(52) U.S. Cl. CPC A61K 39/001102; A61P 37/04; C12N 15/70; C12Q 1/6886; G16B 15/30; A61K 2039/523; C12N 2800/101

(57) ABSTRACT

Engineered Escherichia coli Nissle bacteria comprising one or more heterologous nucleic acids that encode one or more neoantigen epitopes derived from antigenic epitopes present on the surface of a tumor cell and related compositions and methods.

### CROSS-REFERENCE TO RELATED APPLICATIONS

This is a continuation of International Patent Application No. PCT/US2024/024327 filed on 12 Apr. 2024, which claims the benefit of U.S. Provisional Patent Applications Nos. 63/459,400, filed 14 Apr. 2023, 63/541,624, filed 29 Sep. 2023, and 63/552,806, filed 13 Feb. 2024, each of which are hereby incorporated by reference in their entirety.

### STATEMENT REGARDING FEDERALLY SPONSORED RESEARCH OR DEVELOPMENT

This invention was made with government support U01CA247573 awarded by the National Institutes of Health. The government has certain rights in the invention.

### SEQUENCE LISTING

The instant application contains a Sequence Listing which has been submitted in XML format via Patent Center and is hereby incorporated by reference in its entirety. Said XML copy, created on 14 Oct. 2025, is named 39700059USO1SEQLXML, and is 177,733 bytes in size.

### TECHNICAL FIELD OF THE INVENTION

This disclosure generally relates to the fields of personalized medicine, microbiology, immunology, and synthetic biology. More specifically, the disclosure relates to engineered bacteria cells (e.g., E. coli Nissle 1917 bacteria) that contain inter alia at least one neoantigen construct that encode a variable number of neoantigen epitopes related to neoantigens expressed on tumor cells obtained from a subject for delivery to the subject as a vaccine, as well as related compositions and methods.

### BACKGROUND OF THE INVENTION

Synthetic biology is creating the next generation of cancer therapy through the programming of living cells. As bacteria uniquely sense tissue-level signatures of the tumor microenvironment (TME), microbial engineering allows development of intelligent anti-tumor therapeutics. Through optimization of synthetic tumor neoantigen arrays, genetic ablation of microbial machinery, and immunomodulator assimilation, disclosed herein is a microbial tumor neoantigen vaccine which directly modulates the TME and drives therapeutic anti-tumor immunity. The synthetic microbial modifications synergize to achieve enhanced production of tumor neoantigens, susceptibility to blood clearance, and interface with antigen-presenting cells (APCs), as well as ability to drive recombinant antigen-specific cellular immunity.

As described herein, various sets of tumor neoantigens delivered by living anti-tumor vaccines induce potent tumor control and extend survival in advanced murine primary and metastatic solid tumors. Engineered microbes increase local tumor neoantigen density and persist selectively within the TME. The anti-tumor response involves extensive immune education within the TME, with neoantigen-specific activation of CD4⁺ and CD8⁺ T cells, broader activation of both T and B cells, and reduction of immunosuppressive myeloid and regulatory T cell populations. Accordingly, the inventions disclosed herein constitute a new class of therapeutic cancer vaccines, leveraging the advantages of living medicines to deliver arrays of tumor-specific neoantigens within the optimal context to induce effective anti-tumor immunity.

### BRIEF SUMMARY OF THE INVENTION

The present disclosure generally relates to Escherichia coli Nissle bacteria (EcN) that are engineered to comprise one or more heterologous nucleic acids that encode one or more neoantigen epitopes derived from (i.e., related to) neoepitopes that either over-expressed on the surface of tumor cells or are unique to tumor cells (EcNc). In particular embodiments, the heterologous nucleic acids are present in one or more therapeutic vectors.

In some embodiments, neoantigen epitopes comprise between about 5 to 50 amino acids. Exemplary embodiments of such neoantigen epitopes are set forth in SEQ ID NOs: 1-201. In some embodiments, neoantigen epitopes are determined using exome sequencing or transcriptome sequencing of a cancer cell. In some embodiments, neoantigen epitopes are screened for immunosuppressive ability and excluded from the one or more therapeutic vectors. In some embodiments, the heterologous nucleic acids are codon optimized for expression and secretion in EcN.

In some embodiments, each therapeutic vector of the one or more therapeutic vectors comprises one, two, three, four, five, six, seven, eight, nine, ten, or more heterologous nucleic acids that each encode a neoantigen epitope. In some embodiments, the total number of neoantigen epitopes encoded by the heterologous nucleic acids ranges from as few as five to as many as five hundred. In some embodiments, the heterologous nucleic acids are separated by a nucleic acid sequence encoding a spacer. In particular embodiments, the spacer is between three and seven amino acids in length. In specific embodiments, the spacer is four glycine residues and a serine residue (i.e., GGGGS; SEQ ID NO: 202).

In some embodiments, the EcNc are engineered to remove (i.e., lack) cryptid plasmids pMUT1 and pMUT2. In some embodiments, the EcNc are engineered to delete (i.e., lack) one or more proteases. In particular embodiments, the protease is the Ion protease (EcNc^{ΔIon}). In other embodiments, the protease is the OmpT protease (EcNc^{ΔOmpT}). In some embodiments, both proteases are deleted (EcNc^{ΔIon/ΔOmpT}) In some embodiments, the EcNc are engineered to constitutively co-express cytosolic listeriolysin (LLO). In some embodiments, both proteases are deleted and the EcNc are engineered to constitutively co-express listeriolysin (EcNc^{ΔIon/ΔOmpT/LLO+}).

In other embodiments, the present disclosure relates to methods of fabricating a personalized vaccine for a subject, comprising: (a) optionally obtaining a biological sample from the subject, wherein the biological sample comprises one or more nucleic acids derived from a cancer cell present in the subject; (b) identifying one or more potential neoantigens from the one or more nucleic acids by comparing nucleic acid sequences of the one or more nucleic acids to nucleic acid sequences from a non-cancerous cell of the same type; (c) optionally screening peptides comprising the one or more potential neoantigens for an immunogenic response; (d) optionally identifying one or more epitopes on one or more neoantigens that produced an immunogenic response; and (e) transforming a Escherichia coli Nissle bacteria with one or more vectors comprising one or more heterologous nucleic acids that encode one or more neoantigen epitopes, producing the personalized vaccine thereby. In some embodiments, the nucleic acid sequences from a non-cancerous cell of the same type are obtained from a database. In some embodiments, the non-cancerous cell of the same type is obtained from the subject.

In other embodiments, the present disclosure relates to methods of treating a subject, comprising: (a) optionally obtaining a biological sample from the subject, wherein the biological sample comprises one or more nucleic acids derived from a cancer cell present in the subject; (b) identifying one or more potential neoantigens from the one or more nucleic acids by comparing nucleic acid sequences of the one or more nucleic acids to nucleic acid sequences in a non-cancerous cell of the same type; (c) optionally screening peptides comprising the one or more potential neoantigens for an immunogenic response; (d) optionally identifying one or more epitopes on one or more neoantigens that produced an immunogenic response; (e) transforming a Escherichia coli Nissle bacteria with one or more vectors comprising one or more heterologous nucleic acids that encode one or more neoantigen epitopes, producing a personalized vaccine thereby; and (f) administering the personalized vaccine to the subject. In some embodiments, the nucleic acid sequences from a non-cancerous cell of the same type are obtained from a database. In some embodiments, the non-cancerous cell of the same type is obtained from the subject.

In some embodiments, the methods of treatment described herein comprise a second personalized vaccine, comprising (g) obtaining a second biological sample from the subject after the subject exhibits an immune response, wherein the second biological sample comprises one or more tumor-infiltrating lymphocytes (TILs); (h) screening the one or more TILs with one or more peptides comprising the epitopes on one or more neoantigens that produced an immunogenic response; (i) selecting one or more neoantigens, wherein the selected neoantigens are different from the neoantigens in the personalized vaccine; (j) transforming a second Escherichia coli Nissle bacteria with one or more vectors comprising one or more heterologous nucleic acids that encode one or more neoantigen epitopes present on the selected neoantigens; and (k) administering the second personalized vaccine to the subject.

In some embodiments, the EcNc described herein may be administered to a subject or delivered to a tumor in the form of a pharmaceutical composition, which may comprise one or more pharmaceutically acceptable carriers, diluents, or excipients. In some embodiments, the EcNc are formulated as a pharmaceutical composition for intravenous delivery. In some embodiments, the EcNc are formulated as a pharmaceutical composition for intratumoral delivery. In some embodiments, the pharmaceutical composition comprises a plurality of different EcNc strains, wherein each strain expresses a different combination of neoantigen epitopes.

The present disclosure also relates to articles of manufacture useful for treating a tumor. In some embodiments, the articles of manufacture comprise a container comprising EcNc described herein, or pharmaceutical compositions comprising the same, as well as instructional materials for using the same to treat a colorectal tumor. In some embodiments, the articles of manufacture are part of a kit that comprises a bacterial culture vessel and/or bacterial cell growth media.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description of the Invention. This summary is not intended to identify key features or essential features of the claimed subject matter, nor is it intended to be used as an aid in determining the scope of the claimed subject matter.

### BRIEF DESCRIPTION OF THE SEVERAL VIEWS OF THE DRAWINGS

FIGS. 1A-1F provide an overview of microbial tumor neoantigen immunotherapeutics in accordance with the inventions described herein. FIG. 1A: Design of microbial tumor neoantigen vectors, with Circos plot of CT26 mutanome. FIG. 1B:Upper: optimized synthetic neoantigen construct schematic. Middle: relative immunoblot chemiluminescent intensity of neoantigen construct MHCIIa expressed from parent (EcN) vs. derivative strains (n=3 replicates per group). Lower: representative immunoblot of neoantigen construct MHCIIa expression in wildtype EcN, EcNc, EcNc^{ΔIon}, EcNc^{ΔompT}, or EcNc^{ΔIon/ΔompT}. FIG. 1C: Percent GFP⁺ BMDM after incubation with EcN or EcNc^{ΔIon/ΔompT} expressing constitutive GFP (n=3 replicates per group, ****P<0.0001, unpaired student's t-test); Lat. A=Latrunculin A. FIG. 1D: Left: representative image of EcN or EcNc^{ΔIon/ΔompT} spotted on LB agar plate after incubation in human blood, Right: microbial burden quantified as CFU/mL (n=3 replicates per group, **P=0.0039, unpaired student's t-test with Welch's correction). Limit of detection (LOD)=2×10² CFU/mL. FIG. 1E: IL-12p70 quantification in culture supernatants of BMDC pulsed with indicated condition (n=3 replicates per group, **P=0.0018, ****P<0.0001, one-way ANOVA with Tukey's multiple comparisons test). FIG. 1F: Naïve OT-I T cells were incubated with BMDC's pulsed with the indicated condition. Left: IFN-γ quantification in supernatants of OT-I cultures (n=3 replicates per group, ****P<0.0001, one-way ANOVA with Tukey's multiple comparisons test), Middle: IL-2 quantification in supernatants of OT-I cultures (n=3 replicates per group, ****P<0.0001, one-way ANOVA with Tukey's multiple comparisons test). Right: representative histogram depicting CFSE dilution of stimulated OT-I T cells. FIGS. 1B-1F: Data are presented as mean±s.e.m.

FIGS. 2A-2H illustrate the efficacy of microbial tumor neoantigen vectors in primary and metastatic colorectal carcinoma. FIGS. 2A-2D: BALB/c mice with established hind-flank CT26 tumors were treated when average tumor volume was 150-200 mm³. FIG. 2A: Mice received an intratumoral injection of PBS, EcNc^{ΔIon/ΔompT} or EcNc^{ΔIon/ΔompT/LLO+} without neoantigens (NC) or the 3 neoantigen-expressing strain combination (nAg¹⁹) on day 0. Tumor growth curves (n=5-7 mice per group, *P=0.0469, **P=0.0096, ****P<0.0001, ns=not significant (P>0.05), two-way ANOVA with Tukey's multiple comparisons test). FIG. 2B: Mice received an intratumoral injection of PBS, EcNc^{ΔIon/ΔompT} or EcNc^{ΔIon/ΔompT/LLO+}. Intratumoral IL-12p70 levels post treatment (n=6 mice per group, ***P=0.0004, ****P<0.0001, ns=not significant (P>0.05), one-way ANOVA with Tukey's multiple comparisons test). FIG. 2C: Mice received an intravenous injection of EcNc^{ΔIon/ΔompT} NC, EcNc^{ΔIon/ΔompT/LLO+} NC, or EcNc^{ΔIon/ΔompT/LLO+} nAg¹⁹ on day 0 and day 8. Tumor growth curves (n=8-9 mice per group, ****P<0.0001, ns=not significant (P>0.05), two-way ANOVA with Tukey's multiple comparisons test). FIG. 2D: Mice (n=5) received an intravenous injection of EcNc^{ΔIon/ΔompT/LLO+}. Microbial tissue burden quantified as colony-forming units (CFU) per gram of tissue (CFU/g), LOD=1×10³ CFU/g. FIG. 2E: BALB/c mice were treated when average hind-flank CT26 tumor volume was 120 mm³. Mice (n=8 per group) received intravenous injections of PBS, EcNc^{ΔIon/ΔompT/LLO+} OVA, or EcNc^{ΔIon/ΔompT/LLO+} nAg¹⁹, or subcutaneous injections of nAg¹⁹-SLP vaccine every 3-6 days. Tumor growth curves (n=8 mice per group, ****P<0.0001, ns=not significant (P>0.05), two-way ANOVA with Tukey's multiple comparisons test), FIGS. 2F-2H. BALB/c mice were injected intravenously with CT26 Luc cells. Beginning day 4 post tumor cell injection, mice (n=5 per group) received intravenous injections of PBS, EcNc^{ΔIon/ΔompT/LLO+} NC, or EcNc^{ΔIon/ΔompT/LLO+} nAg¹⁹ every 3-5 days. FIG. 2F: Representative images of lung metastases luminescence in each mouse (M1-M5) per group on day 22 post engraftment. FIG. 2G: Mean total flux from lung metastases (n=5 mice per group, ****P>0.0001, ns=not significant (P>0.05), two-way ANOVA with Dunnett's multiple comparisons test). FIG. 2H: Kaplan-Meier survival curve for mice with CT26-Luc lung metastases (n=5 mice per group, **P=0.0017, **P=0.0018, Log-Rank Mantel-Cox test). FIGS. 2A-E, 2G, 2H: Data are presented as mean±s.e.m.

FIGS. 3A-3H illustrate how the microbial tumor neoantigen vaccines drive anti-tumor immunity and remodel the tumor immune microenvironment. FIG. 3A: Immunoblot (anti-6×His) of tumors (n=5) and TDLNs (n=5) from mice (M1-M5) treated intravenously with strain mixture nAg^{19-His} expressing neoantigen constructs MHCIa_{(His6)}, MHCIIa_{(His6)}, and MHCI/II^{v}_{(His6)}. FIGS. 3B-3G: BALB/c mice with established hind-flank CT26 tumors received an intravenous injection of indicated therapeutic or control. 2 days (FIGS. 3B, 3C) or 8 days (FIGS. 3D-3G) after treatment, tumors and TDLNs were extracted. FIG. 3B: Left: Percentage CD80⁺ of cDC1 in TDLNs (n=9 mice per group, *P=0.0189, *P=0.0415, ns=not significant (P>0.05), one-way ANOVA with Tukey's multiple comparisons test). Right: Percentage CD86⁺ of cDC1 in TDLNs (n=9 mice per group, **P=0.0061, ***P=0.0002, ns=not significant (P>0.05), one-way ANOVA with Tukey's multiple comparisons test). FIG. 3C: Left: Percentage CD80⁺ of cDC2 in TDLNs (n=9 mice per group, ****P<0.0001, ns=not significant (P>0.05), one-way ANOVA with Tukey's multiple comparisons test). Right: Percentage CD86⁺ of cDC2 in TDLNs (n=9 mice per group, ***P=0.0001, ****P<0.0001, ns=not significant (P>0.05), one-way ANOVA with Tukey's multiple comparisons test). FIG. 3D: TILs were stimulated with pooled peptides representing the neoantigens encoded in EcNc^{ΔIon/ΔompT/LLO+} nAg¹⁹ in the presence of brefeldin A. Left: Frequency of IFN-γf Foxp3⁻CD4⁺ post-stimulation (n=5 mice per group, *P=0.0238, *P=0.0147, ns=not significant (P>0.05), one-way ANOVA with Tukey's multiple comparisons test). Right: Frequency of IFN-γf CD8⁺ T cells post-stimulation (n=5 mice per group, **P=0.0015, ****P<0.0001, ns=not significant (P>0.05), one-way ANOVA with Tukey's multiple comparisons test). FIG. 3E: TILs were stimulated with PMA and ionomycin in the presence of brefeldin A. Left: Frequency of IFN-γf Foxp3⁻CD4⁺ post-stimulation (n=9 mice per group, *P=0.0461, **P=0.0014, ns=not significant (P>0.05), one-way ANOVA with Tukey's multiple comparisons test). Right: frequency of IFN-γf CD8⁺ T cells post-stimulation (n=9 mice per group, *P=0.0486, **P=0.0040, ****P<0.0001, one-way ANOVA with Tukey's multiple comparisons test). FIG. 3F: Left: Percentage PD-L1⁺ of Ly6G⁺ CD11b⁺ in tumors (n=8-9 mice per group, **P=0.0037, **P=0.0059, one-way ANOVA with Dunnett's multiple comparisons test). Right: Percentage PD-L1⁺ of CD11b⁺ F4/80⁺ in tumors (n=8-9 mice per group, **P=0.0010, ns=not significant (P>0.05), One-way ANOVA with Dunnett's multiple comparisons test). FIG. 3G: Left: Number of Foxp3⁺CD4⁺ T cells per mg tumor (n=9 mice per group *P=0.0131, *P=0.0241, one-way ANOVA with Holm-Šidák's multiple comparisons test). Right: Number of MHCII^{lo+}F4/80⁺ CD11b⁺ macrophages per mg tumor (n=8-9 mice per group, *P=0.0385, *P=0.0407, one-way ANOVA with Dunnett's multiple comparisons test). H. Immunologic mechanism. FIGS. 3B-3G: Data are presented as mean±s.e.m.

FIGS. 4A-4I demonstrate the efficacy of microbial anti-tumor vaccines in orthotopic melanoma. FIG. 4A: Design of microbial neoantigen therapeutics for melanoma. Left: Circos plot depicting mutanome of B16F10. Right: immunoblot of B16F10 neoantigen constructs. FIGS. 4B-4E, 4G-4I: C57BL/6 mice with established hind-flank B16F10 melanoma tumors were treated starting 9 days after tumor engraftment. FIG. 4B: Mice received an intratumoral injection of EcNc^{ΔIon/ΔompT/LLO+} OVA or EcNc^{ΔIon/ΔompT/LLO+} nAg⁴² every 3-5 days. Tumor growth curves (n=7 mice per group, ****P<0.0001, ns=not significant (P>0.05), two-way ANOVA with Šidák's multiple comparisons test). FIG. 4C: Mice received an intravenous injection of either PBS, EcNc^{ΔIon/ΔompT/LLO+} OVA or EcNc^{ΔIon/ΔompT/LLO+} nAg⁴² every 3-5 days. Tumor growth curves (n=5-7 mice per group, ****P<0.0001, ns=not significant (P>0.05), two-way ANOVA with Tukey's multiple comparisons test). FIG. 4D: Kaplan-Meier survival curves for orthotopic B16F10 tumor-bearing mice from (c) (n=5-7 mice per group, ***P=0.0001, Log-rank Mantel-Cox test). FIG. 4E: Mice (n=6 per group) received intravenous injection of the 7-strain combination EcNc^{ΔIon/ΔompT/LLO+} nAg⁴². Microbial tissue burden quantified by CFU per gram of tissue (CFU/g), LOD=1×10³ CFU/g. FIG. 4F: Naïve, tumor free C57BL/6 mice were vaccinated intravenously with the designated treatment. Specific lysis of B16F10-Luc cells by purified splenic T cells from mice (n=5 per group) at specified effector-to-target cell (E:T) ratios (***P=0.001, ****P<0.0001, ns=not significant (P>0.05), one-way ANOVA with Tukey's multiple comparisons test). FIG. 4G: Mice received intravenous injection of either EcNc^{ΔIon/ΔompT/LLO+} OVA or EcNc^{ΔIon/ΔompT/LLO+} nAg⁴² every 3-5 days, intraperitoneal injection of antibody was started 2 days before treatment and every 2-3 days thereafter. Tumor growth curves (n=6-8 mice per group, **P=0.0082, ****P<0.0001, ns=not significant (P>0.05), two-way ANOVA with Tukey's multiple comparisons test). FIGS. 4H-4I: Mice received intravenous injection of either PBS, EcNc^{ΔIon/ΔompT/LLO+} OVA or EcNc^{ΔIon/ΔompT/LLO+} nAg⁴² on day 9 and 12 post orthotopic B16F10 engraftment. FIG. 4H: Left: Number of CD103⁺XCR1⁺ cDC1 cells per mg tumor (n=7-8 mice per group, *P=0.0103, **P=0.0030, ns=not significant (P>0.05), one-way ANOVA with Tukey's multiple comparisons test). Right: Number of CD310b⁺ cDC2 cells per mg tumor (n=7-8 mice per group, **P=0.0038, **P=0.0064, ns=not significant (P>0.05), one-way ANOVA with Tukey's multiple comparisons test). FIG. 4I: Left: Number of Foxp3⁻CD4⁺ T cells per mg tumor (n=7-8 mice per group, **P=0.0015, ***P=0.0008, ns=not significant (P>0.05), one-way ANOVA with Tukey's multiple comparisons test). Right: Number of CD8⁺ cytotoxic T cells per mg tumor (n=7-8 mice per group, **P=0.0022, **P=0.0047, ns=not significant (P>0.05), one-way ANOVA with Tukey's multiple comparisons test). 4B-4I: Data are presented as mean±s.e.m.

FIGS. 5A-5G demonstrate how microbial neoantigen vectors restructure the tumor immune microenvironment and suppress established metastatic melanoma. FIGS. 5A-5D: Mice received an intravenous injection of either PBS, EcNc^{ΔIon/ΔompT/LLO+} OVA or EcNc^{ΔIon/ΔompT/LLO+} nAg⁴² on day 9 and 12 post orthotopic B16F10 engraftment. FIG. 5A: Tumor-infiltrating lymphocytes (TILs) were stimulated with PMA and ionomycin in the presence of brefeldin A. Left: experimental schematic. Middle: Frequency of IFN-γ⁺Foxp3⁻CD4⁺ post-stimulation (n=7-8 mice per group, *P=0.0335, **P=0.0040, ns=not significant (P>0.05), one-way ANOVA with Tukey's multiple comparisons test). Right: frequency of IFN-γ⁺CD8⁺ T cells in tumors (n=7-8 mice per group, ****P<0.0001, ns=not significant (P>0.05), one-way ANOVA with Tukey's multiple comparisons test). FIG. 5B: Left: Frequency of Granzyme-B⁺Foxp3⁻CD4⁺ in tumors (n=7-8 mice per group, **P=0.0024, **P=0.0041, ns=not significant (P>0.05), one-way ANOVA with Tukey's multiple comparisons test). Middle: Frequency of Granzyme-B⁺CD8⁺ T cells in tumors (n=7-8 mice per group, *P=0.0495, **P=0.0014, ns=not significant (P>0.05), one-way ANOVA with Tukey's multiple comparisons test). Right: Frequency of Granzyme-B⁺NK1.1⁺ NK cells in tumors (n=7-8 mice per group, ****P<0.0001, ns=not significant (P>0.05), one-way ANOVA with Tukey's multiple comparisons test). FIG. 5C: Left: Median fluorescence intensity (MFI) of TIM-1 on CD19⁺ B cells in tumors (n=7-8 mice per group, *P=0.0457, **P=0.0029, ****P<0.0001, one-way ANOVA with Tukey's multiple comparisons test). Right: Frequency of TIM-1+CD19⁺ B cells of CD45⁺ cells in tumors (n=7-8 mice per group, *P=0.0442, ns=not significant (P>0.05), one-way ANOVA with Dunnett's multiple comparisons test). FIG. 5D: Left: Frequency of Foxp3⁺CD4⁺ T cells of CD4⁺ cells in tumors (n=7-8 mice per group, **P=0.0035, **P=0.0038, one-way ANOVA with Dunnett's multiple comparisons test). Right: Frequency of MHCII⁻Ly6c⁺ MDSC of CD45⁺ cells in tumors (n=7-8 mice per group, *P=0.0440, ns=not significant (P>0.05), one-way ANOVA with Dunnett's multiple comparisons test). FIGS. 5E-5G: Mice received intravenous injection of either PBS, EcNc^{ΔIon/ΔompT/LLO+} OVA or EcNc^{ΔIon/ΔompT/LLO+} nAg⁴² every 3-5 days starting 2 days after intravenous injection of B16F10-Luc cells. FIG. 5E: Representative images of lung metastases luminescence in each mouse (M1-M5) per group on day 22 post engraftment. FIG. 5F: Mean total flux from systemic metastases (n=5 mice per group, ***P>0.0003, ns=not significant (P>0.05), two-way ANOVA with Dunnett's multiple comparisons test). FIG. 5G: Kaplan-Meier survival curve for mice with B16F10-Luc systemic metastases (n=5 mice per group, **P=0.0015, Log-Rank Mantel-Cox test). FIGS. 5A-5D, 5F, 5G: Data are presented as mean±s.e.m.

FIGS. 6A-6F illustrate neoantigen prediction and synthetic construct design. FIG. 6A: Percentage of predicted CT26 neoantigens containing mutant-epitope(s) with ≤500 nM MHC-I affinity (MHC-I), MHC-II affinity (MHC-II), both MHC-I and MHC-II affinity (Shared), or no epitope meeting affinity criteria (Neither). Previously validated neoantigens within the set are labeled. FIG. 6B:Upper: prototype neoantigen construct design, Lower: immunoblot of EcN expressing prototype neoantigen constructs. FIG. 6C: Upper: immunoblot of EcN expressing prototype neoantigen constructs with or without GS-linkers. Lower: ELISA quantification of neoantigen construct in soluble fraction with or without GS-linkers in DH5a (n=3 per group). NeoAg^{p}=prototype neoantigen construct, G4S1=5-mer GS-linker, pTac^{LO-}=pTac without Lac operator; pTac^{LO+}=with Lac operator. FIG. 6D: Upper: neoantigen construct design with GS-linkers, Lower-left: Relative immunoblot chemiluminescent intensity for prototype construct with or without interspersing glycine-serine linkers (n=6 per group). Lower-right: relative expression of prototype neoantigen construct with GS-linkers under selected promoters (n=12 samples). FIG. 6E: Upper: immunoblot of EcN expressing alternate prototype neoantigen constructs. Lower: ELISA quantification of alternative neoantigen construct in soluble fraction (n=3 per group). Neo^{mE1}=minimal epitope, Neo¹=1 neoantigen LP in construct, Neo²=2 neoantigen LP in construct, G8S2=10-mer GS-linker, CsL=immunoprotease sensitive linker. FIG. 6F: Upper: Immunoblot of neoantigen constructs (NeoAg^{p}, MHCIa, MHCIIa, MHCI/II^{v}), expressed in BL21 or EcN. Lower-left: relative immunoblot chemiluminescent intensity for neoantigen construct expression in EcN vs. BL21 (n=4 per group), Lower-right: relative immunoblot chemiluminescent intensity of predicted neoantigen constructs vs. prototype in BL21 (n=3 samples). FIG. 6C-6F: Data are presented as mean±s.e.m.

FIGS. 7A-7I provide additional evidence of microbial tumor neoantigen vaccine functioning and immunologic activity in vitro. FIG. 7A: Plasmid copy number in wildtype EcN or cryptic plasmid cured EcNc (n=3 per group). FIG. 7B:Upper: relative immunoblot chemiluminescent intensity of synthetic neoantigen construct MHCI/II^{v} expression in wildtype EcN vs. derivative strains (n=3 replicates), Lower: representative immunoblot of construct MHCI/II^{v} expressed in wildtype EcN and derivative strains. FIG. 7C: Upper: relative immunoblot chemiluminescent intensity of synthetic neoantigen construct MHCIa expression in wildtype EcN vs. derivative strains (n=3 replicates), Lower: representative immunoblot of construct MHCIa expressed in wildtype EcN and derivative strains. FIG. 7D: Biofilm formation quantified for wildtype EcN, and derivative strains by crystal violet stain assay. (****P<0.0001, ns=not significant P>0.05, One-way ANOVA with Tukey's multiple comparison test, n=9-12 per group). Median fluorescence intensity (MFI) of H2k^{b}-SIINFEKL complex (FIG. 7E, left) and MHCII (FIG. 7E, right) or CD80 (FIG. 7F, left) and PD-L1 (FIG. 7F, right) for BMDM incubated with the indicated live microbial strain or culture media for 6 hours (*P=0.0231, *P=0.0414, ***P=0.0002, ***P=0.0005, ****P<0.0001, one-way ANOVA with Tukey's multiple comparisons test, n=4 per group). FIG. 7G: Left: sheep red blood cells (RBCs) were incubated with lysate from EcNc^{ΔIon/ΔompT} with (LLO⁺) or without (LLO⁻) cytosolic LLO expression. Absorbance at 541 nm (n=3 per group). Right: percentage of live BMDM after incubation with indicated live microbial strain or control for 6 hours (n=4 per group). FIG. 7H: Immunoblot depicting expression of neoantigen constructs MHCIa, MHCIIa, and MHCI/II^{v} in EcNc^{ΔIon/ΔompT} with (LLO⁺) or without (LLO⁻) co-expression of cytosolic LLO. FIG. 7I: Naïve OT-II T cells were incubated with BMDC's pulsed with the indicated condition. Left: IFN-γ quantification in supernatant of OT-II cultures (****P<0.0001, one-way ANOVA with Tukey's multiple comparisons test, n=3 replicates per group), Middle: IL-2 quantification in supernatant of OT-II culture (****P<0.0001, one-way ANOVA with Tukey's multiple comparisons test, n=3 replicates per group). Right: representative histogram depicting CFSE dilution of stimulated OT-II T cells. FIGS. 7A-7I: Data are presented as mean±s.e.m.

FIGS. 8A-8G characterize intratumoral treatment with microbial tumor neoantigen vaccines. BALB/c mice with established hind-flank CT26 tumors were treated when tumor volume was ˜150-200 mm³. FIGS. 8A-8B: Mice received a single intratumoral injection of EcN WT, EcNc^{ΔIon/ΔompT} or EcNc^{ΔIon/ΔompT/LLO+}. FIG. 8A: Representative image of tumors colonized by microbes with a genome-integrated luminescence cassette. FIG. 8B: Average radiance of microbe colonized tumors in designated groups post intratumoral injection (n=4-5 tumors per group). FIG. 8C: Mice received intratumoral injections of PBS or EcN WT. Tumor growth curves (n=5 mice per group, ns=not significant (P>0.05), two-way ANOVA with Tukey's multiple comparisons test). FIG. 8D: Mice received intratumoral injections of EcN WT without therapeutic expression (NC), expressing construct MHCIa, MHCIIa, or MHCI/II, or EcN nAg¹⁹. Tumor growth curves (n=5 mice per group, ns=not significant (P>0.05), two-way ANOVA with Tukey's multiple comparisons test). FIGS. 8E-8F: Mice (n 5-7 per group) received a single intratumoral injection of PBS, EcNc^{ΔIon/ΔompT} NC, EcNc^{ΔIon/ΔompT} nAg¹⁹, or EcNc^{ΔIon/ΔompT/LLO+} expressing either construct MHCIa, MHCIIa, MHCI/II^{v} or the equal-parts combination of all 3 strains nAg¹⁹. Tumor growth curves (FIG. 8E). and tumor growth rate (FIG. 8F) for the respective treatment group. FIG. 8G: Individual tumor trajectories (n=5-7 mice per group) after intratumoral treatment with PBS or indicated microbial therapeutic. FIGS. 8B-8F: Data are presented as mean±s.e.m.

FIGS. 9A-9D provide a comparative profile of intratumoral treatment with engineered microbial neoantigen vaccines. BALB/c mice with established hind-flank CT26 tumors were treated when average tumor volume was 150-200 mm³. FIGS. 9A-9B: Mice received intratumoral injection of wildtype EcN, EcNc^{ΔIon/ΔompT} or EcNc^{ΔIon/ΔompT/LLO+} either without neoantigen expression (NC) or the strain mixture nAg19 on day 0. FIG. 9A: Relative body weight of CT26 tumor-bearing mice (n=5-7 mice per group, **P=0.0034, ****P<0.0001, ns=not significant (P>0.05), two-way ANOVA with Tukey's multiple comparisons test). FIG. 9B: Mice received intratumoral injection on day 0 and 8. Tumor growth curves (n=7-8 mice per group, **P=0.0020, ****P<0.0001, two-way ANOVA with Tukey's multiple comparisons test). FIG. 9C: Kaplan-Meier survival curves for CT26 tumor-bearing mice (n=7-8 mice per group, **P=0.0061, **P=0.0076, Log-rank Mantel-Cox test). FIG. 9D: Individual tumor trajectories (n=7-8 mice per group) after intratumoral treatment with indicated microbial strain. 9A-9B: Data are presented as mean±s.e.m.

FIGS. 10A and 10B demonstrate the induction of systemic anti-tumor immunity after treatment of a single tumor. BALB/c mice were implanted with CT26 cells on both hind flanks. When average tumor volumes were ˜100-150 mm³, mice received an intratumoral injection of PBS, EcNc^{ΔIon/ΔompT/LLO+} (NC), or EcNc^{ΔIon/ΔompT/LLO+} nAg¹⁹ into a single tumor. FIG. 10A: Tumor growth curves (n=5-6 mice per group, **P=0.0014, ****P<0.0001, two-way ANOVA with Tukey's multiple comparisons test). FIG. 10B: CFU/g of tumor (n=5-6 mice per group), LOD 1×10³ CFU. Data are presented as mean±s.e.m.

FIGS. 11A-11F illustrate the effects of intravenous engineered microbial treatment in primary and metastatic solid tumors. BALB/c mice with established hind-flank CT26 tumors were treated when average tumor volume was ˜120-200 mm³. FIG. 11A: Kaplan-Meier survival curves for CT26-tumor-bearing mice treated with indicated therapeutic (n=8-9 mice per group, ****P<0.0001, **P=0.0021, Log-rank Mantel-Cox test). FIG. 11B: Relative body weight of mice (n=8-9 per group) after intravenous treatment with indicated microbial therapeutic. FIG. 11C: Individual tumor trajectories (n=8-9 mice per group) after intravenous treatment with the indicated microbial therapeutic. FIG. 11D: Mice received an intravenous injection of EcNc^{ΔIon/ΔompT} or EcNc^{ΔIon/ΔompT/LLO+}. Upper: representative luminescent signature of tumors colonized with EcNc^{ΔIon/ΔompT} (LLO−) or EcNc^{ΔIon/ΔompT/LLO+} (LLO+), 48 hours post-injection. Lower: average radiance of colonized tumors (n=7 mice per group). FIGS. 11B-11D: Data are presented as mean±s.e.m. FIGS. 11E-11F: BALB/c mice with established hind-flank CT26 tumors were treated when average tumor volume was ˜120 mm³. nAg¹⁹-SLP was injected subcutaneously. FIG. 11E: Kaplan-Meier survival curves for CT26 tumor-bearing mice treated with indicated therapeutic (n=8 mice per group, **P=0.0055, **P=0.0018, *P=0.0210, Log-rank Mantel-Cox test). FIG. 11F: Individual tumor trajectories (n=8 mice per group) after intravenous treatment with the indicated therapeutic.

FIGS. 12A-12D show the effects of intravenous engineered microbial treatment in metastatic tumors. BALB/c mice were injected intravenously with CT26-Luc cells. FIG. 12A: Upper-left: In vivo, or Upper-right: ex vivo bioluminescent images of mice (n=3) lungs 96-hours post-intravenous injection of CT26-Luc cells. Lower: Histology of metastatic lung foci 96-hours post-intravenous injection of CT26-Luc cells. FIGS. 12B-12D: Every 3-5 days, mice (n=5 per group) received intravenous injection of PBS, EcNc^{ΔIon/ΔompT/LLO+} without therapeutic (NC), or EcNc^{ΔIon/ΔompT/LLO+} nAg¹⁹ starting 4 days after CT26-Luc engraftment. FIG. 12B: Microbial tissue burden quantified as CFU/g tissue, LOD 4×10² CFU (n=3 mice). FIG. 12C: Relative body weight of mice (n=5 per group) after intravenous treatment with indicated therapeutic. FIG. 12D: Individual lung metastases luminescence trajectories (n=5 mice per group). FIGS. 12B, 12C: Data are presented as mean±s.e.m.

FIGS. 13A-13I demonstrate the modulation of the tumor-immune microenvironment by engineered microbial tumor neoantigen vaccines. FIGS. 13A-13F, 13H, 13I: BALB/c mice with established hind-flank CT26 tumors received intravenous injections of indicated therapeutic or control. 2 days (FIG. 13A) or 8 days (FIGS. 13B-13G) after treatment, tumors and TDLNs were extracted. FIG. 13A: Frequency of cDC2 in TDLNs (n=9 mice per group, **P=0.0042, **P=0.0099, ns=not significant (P>0.05), One-way ANOVA with Tukey's multiple comparisons test). FIGS. 13B-13C: Lymphocytes from TDLNs were stimulated ex vivo with PMA and ionomycin in the presence of brefeldin A. FIG. 13B: Left: Frequency of IFN-γ⁺Foxp3-CD4⁺ post-stimulation (n=3 mice per group, *P=0.0313, *P=0.0246, ns=not significant (P>0.05), One-way ANOVA with Tukey's multiple comparisons test). Right: Frequency of TNF-α⁺Foxp3⁻CD4⁺ T cells post-stimulation (n=3 mice per group, *P=0.0445, ns=not significant (P>0.05), One-way ANOVA with Tukey's multiple comparisons test). FIG. 13C: Left: Frequency of IFN-γ⁺CD8⁺ poststimulation (n=3 mice per group, *P=0.0257, ns=not significant (P>0.05), One-way ANOVA with Tukey's multiple comparisons test). Right: Frequency of TNF-α⁺CD8⁺ T cells post-stimulation (n=3 mice per group, **P=0.0017, ***P=0.0008, ns=not significant (P>0.05), One-way ANOVA with Tukey's multiple comparisons test). FIG. 13D: TILs were stimulated with individual 29-mer neoantigen peptides. Number IFN-γ spots (n=8 mice per group, *P=0.0439, *P=0.0364, *P=0.0281, *P=0.0200, Kruskal-Wallis test with Dunn's multiple comparisons test). Data are presented as mean±s.e.m. of background (medium control) subtracted responses. FIG. 13E: TILs were stimulated ex vivo with PMA and ionomycin in the presence of brefeldin A. Frequency of IFN-γ+B220⁺ B cells post-stimulation (n=9 mice per group, *P=0.0351, **P=0.0010, ns=not significant (P>0.05), One-way ANOVA with Tukey's multiple comparisons test). FIG. 13F: Left: Percentage Ki-67⁺ of Foxp3⁻CD4⁺ T cells in tumors (n=9 mice per group, *P=0.0183, ***P=0.0008, one-way ANOVA with Dunnett's multiple comparisons test). Right: Percentage Ki-67⁺ of CD8⁺ T cells in tumors (n=9 mice per group, *P=0.0453, ns=not significant (P>0.05), One-way ANOVA with Dunnett's multiple comparisons test). FIG. 13G: Naïve, tumor free BALB/c mice were vaccinated intravenously with the designated treatment. CT26 was engrafted on a single hind-flank after the final vaccination. Tumor growth curves (n=8 mice per group, ****P<0.0001, two-way ANOVA with Šidák's multiple comparisons test). FIG. 13H: Left: Frequency of FoxP3+CD4⁺ regulatory T cells in tumors (n=9 mice per group, *P=0.0491, **P=0.0072, one-way ANOVA with Holm-Šidák's multiple comparisons test), Right: Frequency of MHCII^{lo+}F4/80⁺ CD11b⁺ macrophages in tumors (n=8-9 mice per group, *P=0.0173, **P=0.0057, one-way ANOVA with Dunnett's multiple comparisons test). FIG. 13I: Left: Percentage PD-L1⁺ of cDCl in TDLN (n=5 mice per group, **P=0.0074, ns=not significant (P>0.05), one-way ANOVA with Dunnett's multiple comparisons test), Right: Percentage PD-L1⁺ of cDC2 in TDLN (n=5 mice per group, *P=0.0103, *P=0.0244, one-way ANOVA with Dunnett's multiple comparisons test). 13A-13C, 13E-13H: Data are presented as mean±s.e.m.

FIGS. 14A-14I concern an assessment of engineered microbial neoantigen therapeutics in B16F10 melanoma. FIG. 14A: Percentage of predicted B16F10 neoantigens containing mutant-epitope(s) with ≤500 nM MHC-I affinity (MHC-I), MHC-II affinity (MHC-II), both MHC-I and MHC-II affinity (Shared), or no epitope meeting affinity criteria (Neither). Previously validated neoantigens within the set are labeled. FIG. 14B: Immunoblot of B16F10 neoantigen construct expression in EcNc^{ΔIon/ΔompT/LLO+}. FIGS. 14C-14I: C57BL/6 mice with established hind flank B16F10 melanoma tumors were treated 9 days after tumor engraftment.

FIGS. 14C-14D: Every 3-5 days, mice received an intravenous injection of PBS, EcNc^{ΔIon/ΔompT/LLO+} OVA, or the 7-strain combination EcNc^{ΔIon/ΔompT/LLO+} nAg⁴². FIG. 14C: Individual tumor trajectories after intravenous treatment with indicated therapeutic (n=5-7 mice per group). FIG. 14D: Relative body weight of B16F10-tumor bearing mice (n=5-7 per group, ns=not significant (P>0.05), two-way ANOVA with Tukey's multiple comparisons test). FIG. 14E: Individual tumor trajectories after intravenous treatment with indicated therapeutic and intraperitoneal treatment with monoclonal antibody. 14F-14I: On day 9 and 12 post-engraftment, B16F10 tumor-bearing mice received an intravenous injection of PBS, EcNc^{ΔIon/ΔompT/LLO+} OVA, or EcNc^{ΔIon/ΔompT/LLO+} nAg⁴². FIG. 14F: Left: Frequency of CD103⁺XCR1⁺ cDC1 in tumors (n=7-8 mice per group, *P=0.0132, ns=not significant (P>0.05), one-way ANOVA with Tukey's multiple comparisons test). Right: Frequency of CD301b⁺ cDC2 in tumors (n=7-8 mice per group, *P=0.0162, ns=not significant (P>0.05), one-way ANOVA with Tukey's multiple comparisons test). FIG. 14G: Left: Frequency of Foxp3⁻CD4⁺ T cells in tumors (n=7-8 mice per group, ***P=0.0001, ****P<0.0001, ns=not significant (P>0.05), one-way ANOVA with Tukey's multiple comparisons test). Right: Frequency of CD8⁺ cytotoxic T cells in tumors (n=7-8 mice per group, **P=0.0097, ***P=0.0002, ns=not significant (P>0.05), one-way ANOVA with Tukey's multiple comparisons test). FIG. 14H: Left: Number of NK1.1⁺ NK cells per mg tumor (n=7-8 mice per group, *P=0.0243, *P=0.0224, ns=not significant (P>0.05), one-way ANOVA with Tukey's multiple comparisons test). Right: Frequency of NK1.1⁺ NK cells in tumors (n=7-8 mice per group, *P=0.0189, *P=0.0389, ns=not significant (P>0.05), one-way ANOVA with Tukey's multiple comparisons test). FIG. 14I: Left: Number of MHCII⁺CD64⁺Ly6c⁺ monocytes per mg tumor (n=7-8 mice per group, **P=0.0041, **P=0.0073, ns=not significant (P>0.05), one-way ANOVA with Tukey's multiple comparisons test). Right: Frequency of MHCII⁺CD64⁺Ly6c⁺ monocytes in tumors (n=7-8 mice per group, *P=0.0230, *P=0.0495, ns=not significant (P>0.05), one-way ANOVA with Tukey's multiple comparisons test). 14D, 14F-14I: Data are presented as mean±s.e.m.

FIGS. 15A-15E. On day 9 and 12 post-engraftment, B16F10 tumor-bearing mice received an intravenous injection of PBS, EcNce^{ΔIon/ΔompT/LLO+} OVA, or EcNc^{ΔIon/ΔompT/LLO+} nAg⁴² FIG. 15A: Left: Percentage CD69⁺ of Foxp3⁻CD4⁺ T cells in tumors (n=7-8 mice per group, *P=0.0228, **P=0.0092, ns=not significant (P>0.05), one-way ANOVA with Tukey's multiple comparisons test). Right: Percentage CD69⁺ of CD8⁺ T cells in tumors (n=7-8 mice per group, **P=0.0021, ****P<0.0001, ns=not significant (P>0.05), one-way ANOVA with Tukey's multiple comparisons test). FIG. 15B: Left: Percentage Ki-67⁺ of Foxp3⁻CD4⁺ T cells in tumors (n=7-8 mice per group, *P=0.0188, **P=0.0020, ns=not significant (P>0.05), one-way ANOVA with Tukey's multiple comparisons test). Middle: Percentage Ki-67⁺ of CD8⁺ T cells in tumors (n=7-8 mice per group, **P=0.0048, **P=0.0086, ns=not significant (P>0.05), one-way ANOVA with Tukey's multiple comparisons test). Right: Percentage Ki-67⁺ of NK1.1⁺ NK cells in tumors (n=7-8 mice per group, *P=0.0366, **P=0.0070, ****P<0.0001, ns=not significant (P>0.05), one-way ANOVA with Tukey's multiple comparisons test). FIG. 15C: Left: Representative histogram of TIM-1 expression on CD19⁺ B cells. Right: Percentage Ki-67⁺ of CD19⁺ B cells in tumors (n=7-8 mice per group, **P=0.0015, ****P<0.0001, ns=not significant (P>0.05), one-way ANOVA with Tukey's multiple comparisons test). FIG. 15D: Frequency of MHC-II^{lo+}F4/80⁺ macrophages in tumors (n=7-8 mice per group, *P=0.0130, ns=not significant (P>0.05), one-way ANOVA with Dunnett's multiple comparisons test). FIG. 15E: Left: MHC-II MFI of CD64⁺Ly6c⁺ monocytes in tumors (n=7-8 mice per group, *P=0.0171, **P=0.0041, ns=not significant (P>0.05), one-way ANOVA with Tukey's multiple comparisons test). Right: MHC-II MFI of CD301b⁺ cDC2 in tumors (n=7-8 mice per group, **P=0.0090, ns=not significant (P>0.05), one-way ANOVA with Tukey's multiple comparisons test). Data are presented as mean±s.e.m.

FIGS. 16A-16C show that microbial neoantigen vaccines suppress metastatic B16F10 melanoma. C57BL/6 mice were injected intravenously with B16F10-Luc cells. FIG. 16A: Upper-left: In vivo, or Upper-right: ex vivo bioluminescent images of mice (n=3) lungs 48-hours post-intravenous injection of B16F10-Luc cells. Lower: Histology of metastatic lung foci 48-hours post-intravenous injection of B16F10-Luc cells. FIGS. 16B-16C: Mice received intravenous injection of either PBS, EcNc^{ΔIon/ΔompT/LLO+} OVA, or nAg⁴² every 3-5 days starting 2 days post intravenous injection of B16F10-Luc cells. FIG. 16B: Individual systemic metastases luminescence trajectories (n=5 mice per group). FIG. 16C: Relative body weight of mice (n=5 per group) after intravenous treatment with indicated therapeutic. Data are presented as mean±s.e.m. Images of systemic metastases luminescence in each mouse not shown.

### DETAILED DESCRIPTION OF THE INVENTION

While the present invention may be embodied in many different forms, disclosed herein are specific illustrative embodiments thereof that exemplify the principles of the invention. It should be emphasized that the present invention is not limited to the specific embodiments illustrated. Moreover, any section headings used herein are for organizational purposes only and are not to be construed as limiting the subject matter described.

Unless otherwise defined herein, scientific, and technical terms used in connection with the present invention shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like.

In addition, ranges provided in the specification and appended claims include both end points and all points between the end points. Therefore, a range of 1.0 to 2.0 includes 1.0, 2.0, and all points between 1.0 and 2.0.

The term “about” as used herein when referring to a measurable value such as an amount, a temporal duration, and the like, is meant to encompass variations of .+−0.20%, .+−0.10%, .+−0.5%, .+−0.1%, or .+−0.0.1% from the specified value, as such variations are appropriate to perform the disclosed methods.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or lists of elements, and, optionally, additional unlisted items. Only terms clearly indicated to the contrary, such as “only one of” or “exactly one of” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either”, “one of”, “only one of”, or “exactly one of”.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art.

The inventions described herein relate to Escherichia coli Nissle bacteria (EcN) that are engineered to comprise one or more heterologous nucleic acids that encode one or more neoantigen epitopes derived from (i.e., related to) neoepitopes that either over-expressed on the surface of tumor cells or are unique to tumor cells (EcNc).

Engineered Escherichia coli Nissle (EcN)

The inventions described herein generally relate to Escherichia coli Nissle bacteria (EcN) that are engineered to comprise one or more heterologous nucleic acids that encode one or more neoantigen epitopes derived from (i.e., related to) neoepitopes that either over-expressed on the surface of tumor cells or are unique to tumor cells (EcNc).

The term “heterologous nucleic acid sequence” refers to a nucleic acid derived from a different organism that encodes for a protein and which has been recombinantly introduced into a cell. In some embodiments, the heterologous nucleic acid sequence is introduced by transformation in order to produce a recombinant bacterial cell. Methods for creating recombinant bacterial cells are well known to those of skill in the art. Such methods include, but are not limited to, different chemical, electrochemical and biological approaches, for example, heat shock transformation, electroporation, liposome-mediated transfection, DEAE-Dextran-mediated transfection, or calcium phosphate transfection. Multiple copies of the heterologous nucleic acid sequence (e.g., between 2 and 10,000 copies) may be introduced into the cell.

In some embodiments, the heterologous nucleic acid sequences are in a plasmid. In some embodiments, the heterologous nucleic acid sequences are in a single operon and are integrated into the genome of the recombinant bacterial cells. In some embodiments, the recombinant bacterial cells comprise at least one exogenous promoter that is in operable linkage with one or more of the heterologous nucleic acid sequences.

As used herein, the term “promoter” means at least a first nucleic acid sequence that regulates or mediates transcription of a second nucleic acid sequence through some manner of operable linkage. A promoter may comprise nucleic acid sequences near the start site of transcription that are required for proper function of the promoter. As an example, a TATA element for a promoter of polymerase II type. Promoters can include distal enhancer or repressor elements that may lie in positions from about 1 to about 500 base pairs, from about 1 to about 1,000 base pairs, from 1 to about 5,000 base pairs, or from about 1 to about 10,000 base pairs or more from the initiation site.

An “operable linkage” refers to an operative connection between nucleic acid sequences, such as for example between a control sequence (e.g., a promoter) and another nucleic acid sequence that codes for a protein, i.e., a coding sequence. If a promoter can regulate transcription of the nucleic acid sequence, then it is in operable linkage with the nucleic acid sequence.

Some aspects of the inventions described herein implicitly relate to culturing the engineered bacterial cells described herein. In some embodiments, a culture comprises the engineered bacterial cells and a medium, for example, a liquid medium, which may also comprise: a carbon source, for example, a carbohydrate source, or an organic acid or salt thereof; a buffer establishing conditions of salinity, osmolarity, and pH, that are amenable to survival and growth; additives such as amino acids, albumin, growth factors, enzyme inhibitors (for example protease inhibitors), fatty acids, lipids, hormones (e.g., dexamethasone and gibberellic acid), trace elements, inorganic compounds (e.g., reducing agents, such as manganese), redox-regulators (e.g., antioxidants), stabilizing agents (e.g., dimethyl sulfoxide), polyethylene glycol, polyvinylpyrrolidone (PVP), gelatin, antibiotics (e.g., Brefeldin A), salts (e.g., NaCl), chelating agents (e.g., EDTA, EGTA), and enzymes (e.g., cellulase, dispase, hyaluronidase, or DNase). In some embodiments, the culture may comprise an agent that induces or inhibits transcription of one or more genes in operable linkage with an inducible promoter, for example doxicycline, tetracycline, tamoxifen, IPTG, hormones, or metal ions. Methods and culture conditions for the generation of microbial cultures are well known to those of skill in the art.

### Therapeutic Methods and Compositions

The inventions described herein also encompass methods of fabricating a personalized vaccine for a subject, comprising: (a) optionally obtaining a biological sample from the subject, wherein the biological sample comprises one or more nucleic acids derived from a cancer cell present in the subject; (b) identifying one or more potential neoantigens from the one or more nucleic acids by comparing nucleic acid sequences of the one or more nucleic acids to nucleic acid sequences from a non-cancerous cell of the same type; (c) optionally screening peptides comprising the one or more potential neoantigens for an immunogenic response; (d) optionally identifying one or more epitopes on one or more neoantigens that produced an immunogenic response; and (e) transforming a Escherichia coli Nissle bacteria with one or more vectors comprising one or more heterologous nucleic acids that encode one or more neoantigen epitopes, producing the personalized vaccine thereby. In some embodiments, the nucleic acid sequences from a non-cancerous cell of the same type are obtained from a database. In some embodiments, the non-cancerous cell of the same type is obtained from the subject.

The inventions described herein also encompass methods of treating a subject, comprising: (a) optionally obtaining a biological sample from the subject, wherein the biological sample comprises one or more nucleic acids derived from a cancer cell present in the subject; (b) identifying one or more potential neoantigens from the one or more nucleic acids by comparing nucleic acid sequences of the one or more nucleic acids to nucleic acid sequences in a non-cancerous cell of the same type; (c) optionally screening peptides comprising the one or more potential neoantigens for an immunogenic response; (d) optionally identifying one or more epitopes on one or more neoantigens that produced an immunogenic response; (e) transforming a Escherichia coli Nissle bacteria with one or more vectors comprising one or more heterologous nucleic acids that encode one or more neoantigen epitopes, producing a personalized vaccine thereby; and (f) administering the personalized vaccine to the subject. In some embodiments, the nucleic acid sequences from a non-cancerous cell of the same type are obtained from a database. In some embodiments, the non-cancerous cell of the same type is obtained from the subject.

In some embodiments, the methods of treatment described herein comprise a second personalized vaccine, comprising (g) obtaining a second biological sample from the subject after the subject exhibits an immune response, wherein the second biological sample comprises one or more tumor-infiltrating lymphocytes (TILs); (h) screening the one or more TILs with one or more peptides comprising the epitopes on one or more neoantigens that produced an immunogenic response; (i) selecting one or more neoantigens, wherein the selected neoantigens are different from the neoantigens in the personalized vaccine; (j) transforming a second Escherichia coli Nissle bacteria with one or more vectors comprising one or more heterologous nucleic acids that encode one or more neoantigen epitopes present on the selected neoantigens; and (k) administering the second personalized vaccine to the subject.

As used interchangeably herein, “treatment” or “treating” or “treat” refers to all processes wherein there may be a slowing, interrupting, arresting, controlling, stopping, alleviating, or ameliorating symptoms or complications, or reversing of the progression of cancer, but does not necessarily indicate a total elimination of all disease or all symptoms. Non-limiting examples of treatment include reducing the rate of growth of a tumor, reducing the size of a tumor, or preventing the metastases of a tumor.

Engineered bacterial cells described herein are preferably administered in one or more therapeutically effective doses. As used herein the terms “therapeutically effective dose” means the number of cells per dose administered to a subject in need thereof that is sufficient to treat the hyperproliferative disorder. In some embodiments, a therapeutically effective dose can be at least about 1×10⁴ cells, at least about 1×10⁵ cells, at least about 1×10⁶ cells, at least about 1×10⁷ cells, at least about 1×10⁸ cells, at least about 1×10⁹ cells, or at least about 1×10¹⁰ cells.

In some embodiments, engineered bacterial cells may be delivered to a subject in the form of a pharmaceutical composition, which may comprise one or more pharmaceutically acceptable carriers, diluents, or excipients. Pharmaceutical compositions may be formulated as desired using art recognized techniques. Various pharmaceutically acceptable carriers, which include vehicles, adjuvants, and diluents, are readily available from numerous commercial sources. Moreover, an assortment of pharmaceutically acceptable auxiliary substances, such as pH adjusting and buffering agents, tonicity adjusting agents, stabilizers, wetting agents, and the like, are also available. Certain non-limiting exemplary carriers include saline, buffered saline, dextrose, water, glycerol, ethanol, and combinations thereof. Pharmaceutical compositions may be frozen and thawed prior to administration or may be reconstituted in WFI with or without additional additives (e.g., albumin, dimethyl sulfoxide). Engineered bacterial cells described herein are preferably formulated for oral, intravenous or intratumoral administration, but other routes of administration known in the art may be utilized.

Particular dosage regimens, i.e., dose, timing, and repetition, will depend on the particular subject being treated and that subject's medical history. Empirical considerations such as pharmacokinetics will contribute to the determination of the dosage. Frequency of administration may be determined and adjusted over the course of therapy and is based on reducing the number of tumor cells or tumor mass, maintaining the reduction of such tumor cells or tumor mass, reducing the proliferation of tumor cells or an increase in tumor mass, or delaying the development of metastasis. A therapeutically effective dose may depend on the mass of the subject being treated, his or her physical condition, the extensiveness of the condition to be treated, and the age of the subject being treated.

### Articles of Manufacture

The inventions disclosed herein also encompass articles of manufacture useful for treating a tumor comprising a container comprising programmable bacterial cells described herein, or a pharmaceutical composition comprising the same, as well as instructional materials for using the same to treat the tumor. In some embodiments, the articles of manufacture are part of a kit that comprises a bacterial culture vessel and/or bacterial cell growth media.

### EXAMPLES

The following examples have been included to illustrate aspects of the inventions disclosed herein. In light of the present disclosure and the general level of skill in the art, those of skill appreciate that the following examples are intended to be exemplary only and that numerous changes, modifications, and alterations may be employed without departing from the scope of the disclosure.

### Example 1

### Cell Lines

The B16F10 (ATCC® CRL-6475) melanoma cell line and CT26 (ATCC® CRL-2638) colon carcinoma cell line were purchased from ATCC®. CT26-Luc cells were lentivirally transduced. Stock and working cell banks were produced, of which fourth to fifth generation passages were used for tumor engraftment. Cells were reauthenticated and confirmed mycoplasma free. Cell culture media and additives were purchased from Gibco. Cells were cultured in incubators at 37° C., with atmosphere of humidified 5% CO₂. B16F10 cells were grown in DMEM supplemented with 10% (vol/vol) FBS, 1× GLUTAMAX™, 1% (vol/vol) MEM Non-essential amino acids solution (Gibco-11140050), and 100 U/mL penicillin-streptomycin. CT26 cells were grown in RPMI-1640 supplemented with 10% (vol/vol) FBS, 1× GLUTAMAX™, 1% (vol/vol) MEM Non-essential amino acids solution (Gibco-11140050), and 100 U/mL penicillin-streptomycin.

### Example 2

### Exome Sequencing

Paired tumor and tail DNA from BALB/c mice bearing subcutaneous CT26 tumors or C57BL/6 mice bearing subcutaneous B16F10 tumors was extracted in triplicate using Qiagen DNEASY® Blood & Tissue Minikit, per manufacturer's instructions. Exome capture from mouse tumor and tail tissue triplicates was conducted using Agilent SureSelect^{XT}. All Exon kit for target enrichment DNA library preparation, according to manufacturer's recommendations (Agilent). Genomic DNA was fragmented by acoustic shearing with a Covaris S220 instrument. Fragmented DNAs were cleaned, end repaired, and adenylated at the 3′-end. Adapters were ligated to DNA fragments, and adapter-ligated DNA fragments enriched with limited cycle PCR. Adapter-ligated DNA fragments were validated using Agilent TAPESTATION® (Agilent) and quantified using QUBIT™ 2.0 Fluorometer (ThermoFisher Scientific) and Real-Time PCR (KAPA Biosystems). Sequencing libraries were clustered onto a lane of a flow cell. After clustering, the flow cell was loaded on an Illumina HISEQ™ 4000 Instrument per manufacturer's instructions. Samples were sequenced using 2×150 bp Paired End configuration. Image analysis and base calling was conducted by the HISEQ™ Control Software. Raw sequence data (.bcl files) generated from Illumina HISEQ™ was converted into fastq files and de-multiplexed using Illumina's bcl2fastq2.17. Sequence reads were trimmed to remove adapter sequences and nucleotides with poor quality. Sequence reads were trimmed to remove adapter sequences and nucleotides with poor quality using Trimmomatic v.0.39. Trimmed reads were aligned to the GRCm38 reference genome using the Illumina DRAGEN™ Bio-IT platform v3.7.5. Alignments were sorted, and PCR/optical duplicates marked, for generation of BAM files. Somatic single nucleotide variants (SNV) and insertion/deletion (indel) variants were called using Illumina DRAGEN™ and GATK Mutect2. All variants from paired-normal tissue and murine variants from dbSNP were removed during the process. VCF files were left aligned and normalized, with splitting of multiallelic sites into multiple sites, using bcftools 1.13. Only tumor-specific variants called by both algorithms were used for further analysis.

### Example 3

### RNA Sequencing

Tumor RNA from BALB/c mice bearing subcutaneous CT26 tumors or C57BL/6 mice bearing advanced subcutaneous primary B16F10 was extracted in triplicate using Qiagen RNEASY® Minikit, per manufacturer's instructions. Extracted RNA samples were quantified using QUBIT™ 2.0 Fluorometer (Life Technologies) and RNA integrity checked using Agilent TAPESTATION® 2400 (Agilent). RNA sequencing libraries were prepared using the NEBNext Ultra RNA library Prep Kit for Illumina per manufacturer's instructions (New England Biolabs). mRNA's were first enriched with Oligo(dT) beads. Enriched mRNAs were fragmented for 15 minutes at 94° C. First strand and second strand cDNAs were synthesized subsequently. cDNA fragments were end-repaired and adenylated at 3′-ends, and universal adapters were ligated to cDNA fragments, followed by index addition and library enrichment by limited-cycle PCR. Sequencing libraries were validated on Agilent TAPESTATION® (Agilent), and quantified using QUBIT™ 2.0 Fluorometer (Invitrogen) and quantitative PCR (KAPA biosystems). Library loading, sequencing, and read trimming were done as for exome sequencing. Trimmed reads were aligned to mm10 reference transcriptome using STAR aligner v.2.5.2b. Unique gene hit counts were calculated using feature Counts from Subread Package v.1.5.2. Unique reads that fell within exon regions were counted. The gene hit counts table was used for expression analysis using DESeq2.

### Example 4

### Neoantigen Prediction and Selection

Somatic VCF files were annotated with mutation-specific RNA expression and allele fraction using Bam-readcount and VAtools, and annotated with the Ensembl Variant Effect Predictor (VEP Ensembl version 104). Only PASS variants from the VCFs were considered. Annotated VCFs were analyzed using pVacSeq for neoepitope discovery. MHC-I affinities were predicted with NetMHCpan version 4.1 and NetMHC version 4.1, while MHC-II affinities were predicted with NetMHCIIpan version 4.1 and NNalign version 2.0. Both median and best predicted affinities were considered in neoantigen selection for construct design. Exonic mutations based on single-nucleotide polymorphisms or insertions/deletions with predicted neoantigens were selected and classified based on the following criteria: 1) present in all tumor sample triplicates and none of normal tissue triplicates (DNA-VAF≥0.05), 2) non-synonymous mutation resulting from either SNP or indel, 3) confirmed exonic mutation transcription (RNA-VAF≥0.05) and gene expression by RNA-sequencing in tumor sample triplicates (TPM≥1), 4) mutant-residue containing LP with at least 1 epitope of predicted MHC-I or MHC-II IC₅₀≤6000 nM. Predicted neoantigens fulfilling all prior constraints and which satisfied the following Summative Affinity Index (SAI) calculation were considered for selection and incorporation into therapeutics:

$$1 - ( \frac{MHCI {IC}_{50} WT ( nM )}{MHCI {IC}_{50} MT ( nM )} ) + 1 - ( \frac{MHCII {IC}_{50} WT ( nM )}{MHCII {IC}_{50} MT ( nM )} ) < 0$$

Where MHC-I IC₅₀ MT and WT are the predicted MHC-I IC₅₀ of the highest affinity MHC-I binding mutant and corresponding wildtype epitope, and MHC-II IC₅₀ MT and WT are the predicted MHC-II IC₅₀ of the highest affinity MHC-II binding mutant and corresponding wildtype epitope contained within the neoantigen-derived LP.

### Example 5

### Strains and Plasmids

Plasmids were constructed using restriction-enzyme mediated and Gibson assembly cloning methods. Neoantigen construct iterations were designed and created as Geneblocks (IDT) encoding a constitutive promoter with 5′-UTR containing ribosome-binding site computationally selected using The Ribosome Binding Site Calculator, followed by coding region comprised of mutant-reside containing LPs connected in tandem or by various described linkers. 5′-BamHI and 3′-XbaI restriction endonuclease sites were added to all constructs. Coding sequences were codon optimized for E. coli. Constructs were cloned between BamHI and XbaI restriction sites on a stabilized p246-luxCDABE plasmid where luxCDABE had been cloned out, and flanked by 3′-λ-phage transcription terminator, with high-copy pUC origin. For protein expression assessment studies, a 6×-histidine tag was added at the C-terminal before the stop-codon of the neoantigen construct coding sequence by PCR amplification of full construct plasmids with oligonucleotide containing 6×-histag sequence followed by kinase, ligase, DpnI enzyme mix protocol (NEB). Neoantigen construct plasmids were transformed into chemically competent E. coli DH5α or BL21(DE3) (New England Biolabs), or electrocompetent EcN parental strain and genetic derivatives. The parental EcN strain and derivatives used in this study bear an integrated luciferase cassette within the genome. Plasmid encoding LLO was constructed by cloning in hok/sok stabilization system to pCG02-p15a backbone, PCR amplification of backbone with SLC cloned out, and Gibson assembly of Geneblock encoding LLO under constitutive promoter and backbone. Constitutive LLO plasmids were transformed into electrocompetent EcN parental and genetic derivative strains. Strains were cultured in lysogeny broth (LB) medium with antibiotics for plasmid retention (pUC:kanamycin 50 g/mL; p15a:spectinomycin 100 g/mL) in a 37° C. orbital incubator.

### Example 6

### Construction of Cryptic Plasmid Cured EcN

Cryptic plasmids in EcN were cured with Cas9-mediated double-strand break. EcN was transformed with pFREE or pCryptDel4.8 to cure pMUT1 or pMUT2, respectively. The transformants were grown overnight and diluted into 1:1000 the next day with rhamnose and anhydrotetracycline. After 24 hours of incubation, the culture was streaked on the LB plate without antibiotics. The colonies were screened with colony PCR for verification of the loss of cryptic plasmids.

### Example 7

### Construction of Genetic Knockout Strains

Gene knockout was carried out with the lambda red recombination system. In brief, EcNc was transformed with pKD46. Transformants were made electrocompetent by growing at 30° C. with ampicillin and L-arabinose. The chloramphenicol resistance cassette with corresponding overhangs for each target gene deletion was prepared by PCR amplification of pKD3. Electroporation was performed using 100 μL of competent cells and 50-300 ng of amplified DNA. After 2 hours of recovery, cells were plated on LB with 17 μg/mL chloramphenicol and incubated at 37° C. overnight. The next day, gene deletion was verified by colony PCR. For the excision of the antibiotic resistance marker, pCP20 was transformed, plated on LB with ampicillin, and incubated at 30° C. overnight. The clone was then cultured at 43° C. overnight for the induction of flippase and plasmid curing. Antibiotic resistance marker-free clones were screened for the second round of gene deletion.

### Example 8

qPCR for Plasmid Copy Number

Copy number variant plasmids were constructed from the pUC-GFP plasmid. This backbone plasmid was PCR-amplified and Gibson assembled with sc101*, p15A, or ColE1 origin of replication insert. The respective inserts were prepared from PCR amplification of template plasmid pCG02_sc101*, pCG02_p15A, or pTH05. The plasmid copy number (PCN) was determined by the relative abundance of plasmid DNA compared to the genomic DNA measured with qPCR. The strains with the plasmid of interest were grown at 37° C. overnight. The next day, cells were harvested by centrifugation at 3,000 g for 10 minutes and resuspended in distilled water to make the OD into 1. Resuspended cells were then further 5-fold serially diluted and used for generating a standard curve for determining PCR efficiency, E. 25-fold diluted samples were used for the measurement of Ct values. Samples were denatured at 95° C. for 10 min and 2 μL was added into 18 μL of NEB Luna Universal qPCR Master Mix in each well of the 96-well reaction plate. E was defined from the slope, S, of each standard curve with the equation E=5(−1/S), and plasmid copy number (PCN) was determined with the equation:

$$PCN = \frac{{E}_{G}^{{Ct}_{\text{?}}}}{{E}_{P}^{{Ct}_{\text{?}}}}$$

$$\text{?} \text{indicates text missing or illegible when filed}$$

where respective values for genomic DNA are denoted by subscript G and plasmid DNA by subscript P.

### Example 9

### Immunoblot and ELISA

Strains expressing neoantigen constructs with 6×-HisTag located on C-terminus of protein were grown overnight in LB media with appropriate antibiotics. Optical density (OD) measurement at 600 nm (OD₆₀₀) was used to match CFU concentration between all cultures. Microbial CFU concentration-matched cultures were centrifuged at 3000 rcf at 4° C. for 10 minutes, and resuspended in PBS. Samples were sonicated on ice for 2 minutes with 5-second on/5-second off pulses. Sonicated samples were centrifuged at 10,000 rcf for 20 minutes at 4° C. to separate soluble and insoluble fractions, or complete lysate used directly. Sample fractions were mixed with SDS-loading buffer with 5 mM dithiothreitol, boiled, and subject to immunoblot analysis. For relative quantification of immunoblot chemiluminescent intensity, all target proteins bands on the same blot were normalized to the loading control band DnaK for each respective sample. Normalized values were then divided to provide relative intensity values. Mouse anti-6×His (aTHE) was purchased from Genscript, mouse anti-DnaK was purchased from Abcam (8E2/2). For histidine-tag ELISA, soluble sample fractions were analyzed using GenScript His Tag ELISA Detection Kit, per manufacturer's instructions. For in vivo immunoblot analysis, BALB/c mice bearing a 12-day established hind-flank CT26 tumor were injected intravenously with 100 μL of 5×10⁷ CFU/mL mixture of EcNc^{ΔIon/ΔompT/LLO+} strains expressing neoantigen constructs MHCIa, MHCIIa, and MHCIII^{v} that all had a C-terminal 6×-histidine tag before the stop codon. 48 hours after treatment, tumor and TDLN were extracted from five mice and placed in B-PER lysis reagent (ThermoFisher Scientific) with 250 U/mL benzonase nuclease (Millipore Sigma), and homogenized using a GENTLEMACS® tissue dissociator (Miltenyi Biotec; C-tubes). Tissue homogenate was then sonicated on ice for 3 minutes with 5-second on/5-second off pulses. Sonicated samples were centrifuged at 10,000 rcf for 20 minutes at 4° C. to separate soluble and insoluble fractions. Sample fractions were mixed with SDS-loading buffer with 5 mM dithiothreitol, boiled, and subject to immunoblot analysis.

### Example 10

### Blood Bactericidal Assay

EcN cryptic plasmid cured wildtype (EcNc WT) or double protease mutant (EcNceA^{on}l^{ΔOmpT}) were cultured overnight in LB media. Cultures were centrifuged at 3000 rcf for 10 minutes, and resuspended in 1 mL of ice-cold sterile PBS. Cultures were normalized to OD₆₀₀ of 1 in sterile PBS. 50 μL of OD₆₀₀ microbes was added to 1 mL of single donor human whole blood (Innovative Research) in triplicate and incubated in a 37° C. stationary incubator. At various time points, a sample was taken from each and serial dilution prepared in PBS. The dilutions were plated on LB agar with erythromycin. After incubation overnight at 37° C., colonies were counted.

### Example 11

### Biofilm Assay

Microbial biofilm formation assays were conducted as described previously. Briefly, EcN wildtype, cryptic plasmid cured (EcNc), Ion mutant (EcNc^{ΔIon}), OmpT mutant (EcNc^{ΔOmpT}) or double protease mutant (EcNc^{ΔIon/ΔOmpT}) were cultured for 48 hours in LB media with 50 μg/mL erythromycin in borosilicate glass tubes in a 30° C. stationary incubator, with tubes caps wrapped tightly with parafilm to prevent evaporation. At 48 hours, cultures were discarded and borosilicate tubes washed 3-times with PBS. Tubes were inverted and let dry for 6-hours. Biofilms left on borosilicate tubes were stained with 0.1% (vol/vol) crystal violet for 15 minutes. Crystal violet stain was discarded, tubes were washed 3-times with PBS, then inverted and let dry overnight. Crystal violet-stained biofilms were dissolved with 95% ethanol, and transferred to 96-well plates for measurement of absorbance at 590 nm.

### Example 12

### Phagocytosis Assay

Bone marrow derived macrophages (BMDM) from BALB/c or C57BL/6 mice were cultured on 15-cm non-tissue culture treated Petri dishes, in RPMI with 20% FBS, 25 ng/mL M-CSF (R&D Systems), and 100 U/mL penicillin-streptomycin. Media was replaced with fresh media after 4 days of culture. After 7 days in culture, BMDM plates were washed with PBS and adherent macrophages were dissociated using trypsin-EDTA. Macrophages were washed in PBS, resuspended at a density of 2×10⁵/mL in media, and 1 mL transferred to each well of 24-well plates. 24-well plates were incubated overnight in a 37° C. incubator with humidified 5% CO₂. EcN wildtype or cryptic plasmid cured, double protease mutant (EcNc^{ΔIon/ΔOmpT}), with or without constitutive GFP-expressing plasmid were cultured overnight in LB media with appropriate antibiotics. Cultures were centrifuged at 3000 rcf for 10 minutes, washed 3-times with PBS, and resuspended at a density of 4×10⁸ bacteria/mL in sterile PBS. Media from wells in 24-well plate containing macrophages was aspirated, wells washed three times with PBS, and 1 mL of RPMI with 10% mouse serum, without antibiotics was added to each well. Latrunculin A was added at a concentration of 1 μM to selected wells to inhibit phagocytosis. 2×10⁷ microbes were added to each well, with each condition replicated in triplicate. Microbial strains were incubated with BMDMs for 30-minutes, in a 37° C. incubator at 20 rpm. After 30 minutes, media was aspirated and wells were washed 6-times with PBS. Adherent macrophages were dissociated using non-enzymatic cell dissociation buffer (Gibco), and analyzed by flow cytometry.

### Example 13

### In Vitro BMDM Activation

BMDM and bacteria were cultured as described above. Briefly, BMDM were washed in PBS, resuspended at a density of 2×10⁵/mL in media, and 1 ml transferred to each well of 24-well plates. 24-well plates were incubated overnight in a 37° C. incubator with humidified 5% CO₂. EcN wildtype (EcN) or cryptic plasmid cured, double protease mutant (EcNc^{ΔIon/ΔOmpT}) with constitutive OVA expression on a pUC origin plasmid were cultured overnight in LB media with appropriate antibiotics. Cultures were centrifuged at 3000 rcf for 10 minutes, washed 3-times with PBS, and resuspended at a density of 4×10⁸ bacteria/mL in sterile PBS. Media from wells in 24-well plate containing macrophages was aspirated, wells washed gently 3-times with PBS, and 1 mL of RPMI with 10% mouse serum without antibiotics was added to each well. 5×10⁶ live microbes were added to each well, with each condition replicated in triplicate. Live microbial strains were incubated with BMDMs for 6-hours in a 37° C. incubator. After 6-hours, media was aspirated and wells washed 6-times with PBS. Adherent macrophages were dissociated using non-enzymatic cell dissociation buffer (Gibco), and analyzed by flow cytometry. DRAQ7 cell viability reagent was used to exclude dead cells (diluted 1:1000 in FACS buffer). Extracellular antibodies for myeloid activation panel included CD80 (16-10A1, Biolegend), MHC-II (M5/114.15.2, Biolegend), PD-L1 (10F.9G2, Biolegend), and H2K^{b}-SIINFEKL (25-D1.16, Biolegend).

### Example 14

### BMDC Stimulation In Vitro

Bone marrow derived dendritic cells from C57BL/6 mice were cultured on 15-cm non-tissue culture treated Petri dishes, in RPMI with 20% FBS, 20 ng/mL GM-CSF (Biolegend), and 100 U/ml penicillin-streptomycin. Every 1-2 days for the first four days, plates were gently washed and non-adherent granulocytes removed by aspirating 50% of the culture media, with replacement of fresh media each time. On day four, media was gently aspirated and replaced with fresh culture media with 20 ng/mL GM-CSF. On day six, BMDC plates were washed with PBS and loosely adherent and non-adherent cells were harvested by washing with PBS. Cells were centrifuged at 300 rcf×5 minutes, resuspended in fresh culture media, and replated on 15-cm non-tissue culture treated Petri dishes. On days seven and eight, plates were washed with PBS and loosely adherent and non-adherent cells were harvested by washing with PBS. Cells were centrifuged at 300 rcf×5 min, resuspended in fresh culture media at a density of 2.5×10⁵/mL, and 200 μL transferred to 96-well plates and incubated overnight in a 37° C. incubator. The next day, media from wells containing BMDCs was aspirated, and 1 mL of RPMI with 10% mouse serum, without antibiotics was added to each well. BMDCs were pulsed with bacterial strains at an MOI of 10 for 2-hours, plates were centrifuged at 300 rcf×5 minutes, media aspirated and replaced with fresh RPMI with 10% FBS and 30 g/mL gentamicin and 100 U/mL penicillin-streptomycin. Plates were incubated for 48 hours in a 37° C. incubator, after which time supernatant was assessed for IL-12p70 using ELISA (Mouse IL-12p70 QUANTIKINE® ELISA Kit, R&D systems) according to the manufacturer's instructions.

### Example 15

### OT-I and OT-II T Cell Stimulation and Proliferation

BMDC were cultured as above, resuspended at a density of 2.5×10⁵/mL, and 5×10⁴ BMDC transferred to 96-well plates and incubated overnight in a 37° C. incubator. The next day, media from wells containing BMDCs was aspirated, and 1 mL of RPMI with 10% mouse serum, without antibiotics was added to each well. BMDCs were pulsed with 2×10⁶ CFU of the respective bacterial strain for 2.5-hours, plates were centrifuged at 300 rcf×5 minutes, media aspirated and replaced with fresh RPMI with 10% FBS and 30 g/mL gentamicin and 100 U/mL penicillin-streptomycin. Spleens from naïve OTI and OTII mice were extracted, filtered through 100 μm cell strainers and washed in complete RPMI. OTI and OTII T cells were isolated from single-cell suspensions of the spleens of the respective transgenic mice using the EASYSEP™ Mouse T Cell Isolation Kit (StemCell Technologies) according to the manufacturer's instructions. Purified OT-I and OT-II T cells were resuspended in T cell media (RPMI-1640 supplemented with 10% (vol/vol) FBS, 1× GLUTAMAX™, 1% (vol/vol) MEM Non-essential amino acids solution (Gibco-11140050), 5 μM β-mercaptoethanol, and 100 U/mL penicillin-streptomycin) at a density of 5×10⁵/mL and 5×10⁴ T cells incubated with BMDC pulsed with the respective microbial strains. For cytokine secretion assessment, T cells were incubated with BMDC for 24 hours, after which time supernatant was assessed for IFNγ and IL-2 using ELISA (Mouse IFNgamma QUANTIKINE® ELISA Kit, Mouse IL-2 QUANTIKINE® ELISA Kit, R&D systems) according to the manufacturer's instructions.

For CFSE proliferation assays, 1×10⁷ OT-I or OTII T cells were resuspended in 1 mL of room temperature PBS, and 1 μL of 5 mM CFSE (Biolegend) was added to cells while vortexing. T cells were incubated in CFSE solution for 5 minutes at room-temperature protected from light, after which time the staining was quenched by adding 10-times staining volume of cell culture media. T cells were centrifuged at 300 rcf×5 minutes, resuspended in T cell media at a density of 5×10⁵/mL and incubated for an additional 10 minutes. 5×10⁴ T cells were incubated with 5×10⁴ BMDC pulsed with the respective microbial strains for 96 hours. At 72 hours, 50% of media from each well was gently aspirated and replaced with fresh T cell media. At 96 hours, OT-I and OT-II T cells were harvested and CFSE staining assessed via flow cytometry.

### Example 16

### Listeriolysin Hemolytic Activity Assay

Bacteria were grown overnight in LB containing appropriate antibiotics. Cultures were centrifuged at 3000 rcf for 10 minutes and 10 OD of bacteria resuspended in 0.1% BSA in PBS titrated to pH of 5.3 with 1M HCl. Bacteria were sonicated for 2 minutes with 5 second on/4-second off pulses. After sonication the soluble fraction was isolated by centrifugation at 10000 rcf for 20 minutes at 4° C. Sheep RBCs were washed 3-times with PBS and resuspended at a final concentration of 6×10⁸/mL in 0.1% BSA in PBS titrated to pH of 5.3. 500 μL bacterial supernatant was added to 500 μL sheep RBC suspension and incubated for 15 minutes at 37° C. After incubation RBC mixtures were centrifuged at 1000 rcf for 1 minute, and absorbance of supernatant at 541 nm measured.

### Example 17

### Animal Experiments

All animal experiments were approved by the Institutional Animal Care and Use Committee (Columbia University, protocol AABQ5551). Male and female 6-7-week-old BALB/c (Taconic) and C57BL/6 (Taconic) mice were kept in accordance with rules for animal research at Columbia University. For subcutaneous tumor models, either 5×10⁶ CT26 cells in 100 μL were inoculated subcutaneously on the hind-flank of BALB/c mice, or 5×10⁵ B16F10 melanoma cells on the hind flank (orthotopic) of C57BL/6 mice, using a 26G-needle on a 1 cc syringe. CT26 tumors were allowed to establish for 12 days or until average tumor volume reached 200 mm³ as indicated for each experiment, and mice distributed between groups to equate average starting tumor volume in all groups before initial treatment. B16F10 tumors were allowed to establish for 10 days, and initial tumor volume equated between groups in the same manner before treatment. Tumor sizes were measured unblinded with a caliper every one to three days for calculating tumor volumes using the equation (a²×b)/2 (a=width, b=length). Group tumor sizes were computed as mean±s.e.m. Bodyweight was measured each time tumor measurements were taken. Animals were euthanized when either: exhibiting clinical signs of impaired health, tumor burden of 2 cm for subcutaneous tumors, 20% body weight loss, or as otherwise recommended by veterinary staff.

For lung metastases model, 6×10⁵ CT26-Luc cells were slowly injected in 100 μL sterile PBS through the lateral tail vein with a 27 G-needle on 1 cc syringe. Lung metastases were allowed to establish for 6 days prior to treatment. Mice were randomly distributed between groups for lung metastases experiments after lung-metastases engraftment. For in vivo luminescence tracking of lung metastases burden, mice were injected i.p. with 150 μL aqueous solution of D-Luciferin (1 mg/mL) 7-minutes prior to imaging then placed under isoflurane anesthesia for imaging using an in vivo imaging system (IVIS). Total flux from lungs using bioluminescence from region of interest (ROI) was used to quantify tumor burden. For all tumor experiments, subcutaneous or metastases models, a minimum of 5 mice per group were used.

### Example 18

### Microbial Administration for In Vivo Experiments

For therapeutic administration, strains were grown overnight in LB media containing the specific antibiotics. Overnight cultures were centrifuged for 10-minutes at 3000 rcf and washed 3-times with ice-cold, sterile PBS. Microbes were delivered intratumorally at a concentration of 2.5×10⁸ CFU/mL in PBS, with 20 μL injected using a 1 cc syringe with 29 G-needle. For intravenous treatment, 100 μL of microbes were delivered at a concentration of 5×10⁷ CFU/mL, through the lateral tail vein using a 1 cc syringe with 29 G-needle.

### Example 19

### Biodistribution and In Vivo Bacterial Dynamics

For biodistribution experiments, BALB/c mice bearing a 12-day established hind-flank CT26 tumor were injected intravenously with 100 μL of 5×10⁷ CFU/mL EcNc^{ΔIon/ΔOmpT}. 72-hours after injection, tumors, and organs (TDLN, lungs, spleen, liver, heart, kidneys) were extracted from 5 mice, weighed, and homogenized using a GENTLEMACS™ tissue dissociator (Miltenyi Biotec; C-tubes). Homogenates were serially diluted and plated on LB-agar plates at 37° C. overnight. Colonies were quantified per organ and computed as CFU per gram of tissue. For tracking bacterial colonization of subcutaneous tumors, tumor bearing mice treated intratumorally or intravenously with EcN parental strain or derivates were imaged using IVIS at various time points.

### Example 20

### Flow Cytometry Immunophenotyping

Tumors and TDLN were extracted for immunophenotyping on day 8 after intravenous treatment with vehicle control (PBS) or respective microbial strain(s). Lymphoid and myeloid immune subsets were isolated from tumor tissue by mechanical homogenization of tumor or TDLN tissue, followed by digestion with collagenase A (1 mg/mL; Roche) and DNase I (0.5 μg/mL; Roche) in isolation buffer (RPMI 1640 with 5% FBS, 1% L-glutamine, 1% penicillin-streptomycin, and 10 mM HEPES) for 1-hour at 37° C. for tumors or 30 minutes at 37° C. for TDLN, on a shaker platform at 150 rpm. For ex vivo lymphocyte stimulation with PMA and ionomycin, TDLN were not digested prior to use. Tumor and TDLN homogenates were filtered through 100 μm cell strainers and washed in isolation buffer.

To measure cytokine production by T cells, cells were stimulated for 3-hours with PMA (50 ng/mL; Sigma-Aldrich) and ionomycin (1 nM; Calbiochem) in the presence of brefeldin A (1 g/mL). To measure neoantigen-specific cytokine production by T cells, cells were stimulated for five hours with pools of peptides (2 g/mL) representing the neoantigens encoded in therapeutic strains in the presence of brefeldin A (1 g/mL). Cells were stained in FACS buffer (PBS containing 2% FBS, 2 mM EDTA, and 0.09% sodium azide). Ghost Dye cell viability reagent was used to exclude dead cells (diluted 1:1000 in PBS). Extracellular antibodies for lymphoid immunophenotyping included: CD4 (RM4-5, Biolegend), NKp46 (29A1.4, BD Biosciences), CD45 (30-F11, BD Biosciences), B220 (RA3-6B2, BD Biosciences), CD8a (53-6.7, Tonbo). After extracellular staining, cells were washed with FACS buffer, and fixed using the FOXP3/transcription factor staining buffer set (Tonbo), as per manufacturer's instructions. Intracellular antibodies for lymphoid immunophenotyping included: Foxp3 (FJK-16s, eBioscience), CD3ε (145-2C11, Tonbo), TCRβ (H57-507, BD Biosciences), Ki67 (SolA15, eBioscience), TNFα (MP6-XT22, eBioscience), and IFN7 (XMG1.2, Tonbo). For myeloid immunophenotyping, extracellular antibodies included: Ly6C (HK1.4, Biolegend), I-A/I-E (M5/114.15.2, Biolegend), XCR1 (ZET, Biolegend), CD11b (M1/70, Biolegend), CD103 (M290, Biolegend), CD45 (30-F11, BD Biosciences), F4/80 (BM8.1, Tonbo), CD11c (B418, Tonbo), CD172a/SIRPα (P84, Biolegend), Ly6G (1A8, Biolegend), and PD-L1 (10F.9G2, Biolegend). After staining, cells were washed and resuspended with FACS buffer for flow cytometry analysis using a BD LSRFORTESSA™ cell analyzer. FACS Diva software was used for data acquisition. Collected flow cytometry data were analyzed using FLOWJO®.

### Example 21

### Synthetic Peptides

Synthetic peptides representing neoantigen sequences for lymphocyte restimulation assays were synthesized by and purchased from Peptide 2.0. All peptides were ≥95′(purty.

### Example 22

### Statistics

Statistical analyses were performed using GRAPHPAD PRISM® 9. For each experiment, the particular statistical analysis is detailed in the respective figure legend. Unpaired Student's T-test, one-way ANOVA, or two-way ANOVA with appropriate post-hoc test was used for data that were approximately normally distributed. For analysis of Kaplan-Meier survival experiments, a log-rank (Mantel-Cox) test was used.

### Example 23

### Neoantigen Construct Design and Fabrication

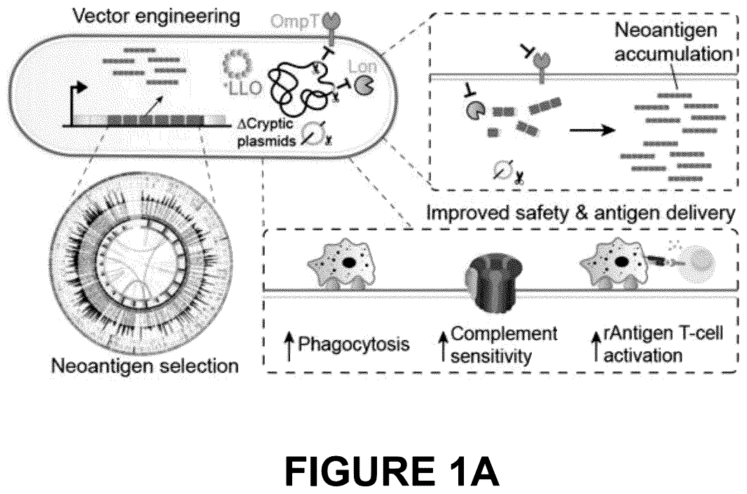

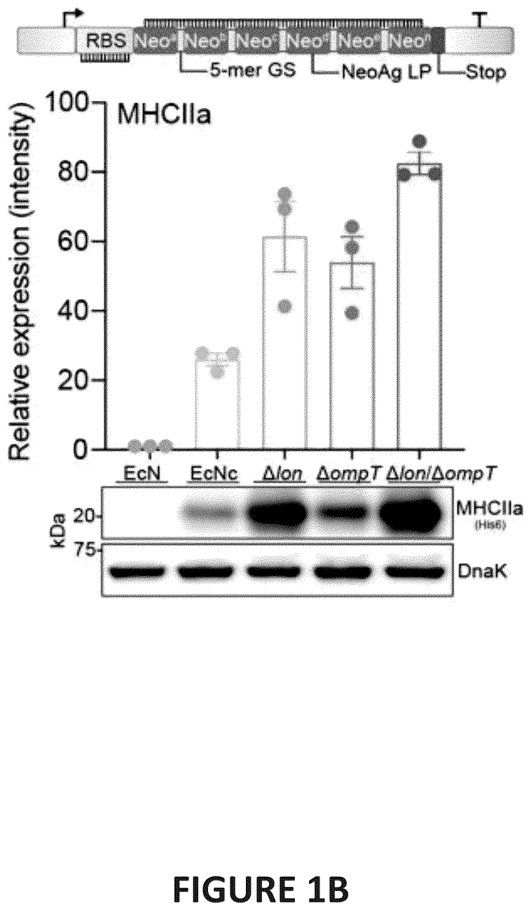

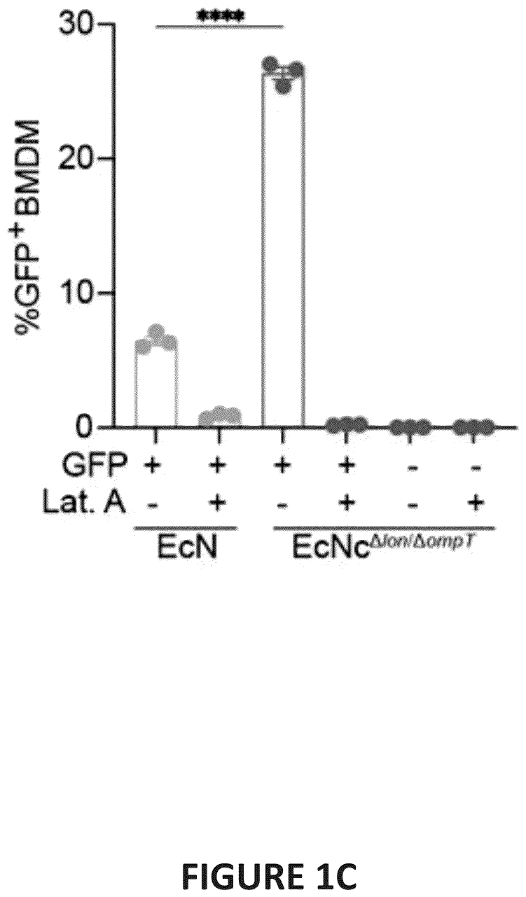

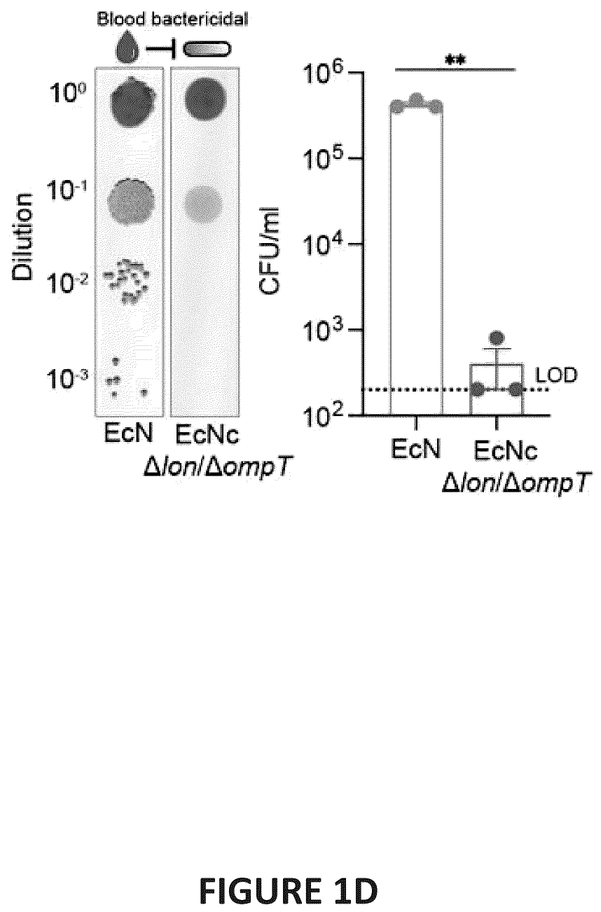

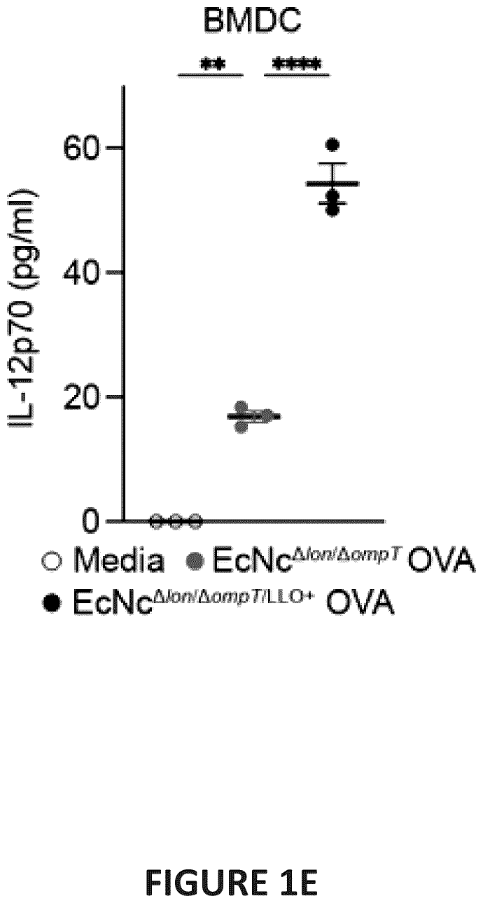

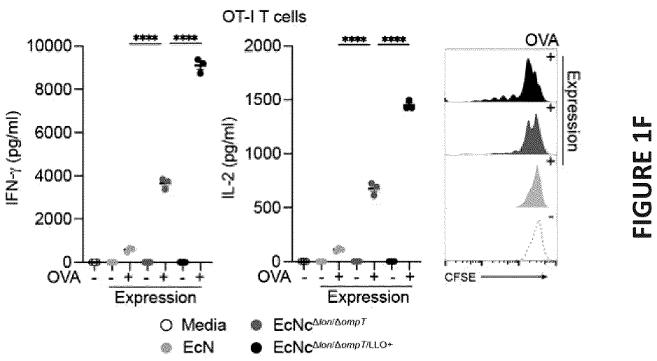

To enable effective cancer vaccination, an engineered bacterial system was developed in probiotic EcN to enhance expression, delivery, and immune-targeting of arrays of tumor exonic mutation-derived epitopes highly expressed by tumor cells and predicted to bind major histocompatibility complex (MHC) class I and II (FIG. 1A). This system incorporates several key design elements that enhance therapeutic utility: (1) optimization of synthetic neoantigen construct form with (2) removal of cryptic plasmids and deletion of Lon and OmpT proteases to increase neoantigen accumulation, (3) increased susceptibility to phagocytosis for enhanced uptake by antigen-presenting cells (APCs) and presentation of MHC class II-restricted antigens, (4) expression of Listeriolysin O (LLO) to induce cytosolic entry for presentation of recombinant encoded neoantigens by MHC class I molecules and Th1-type immunity, and (5) improved safety for systemic administration due to reduced survival in the blood and biofilm formation.

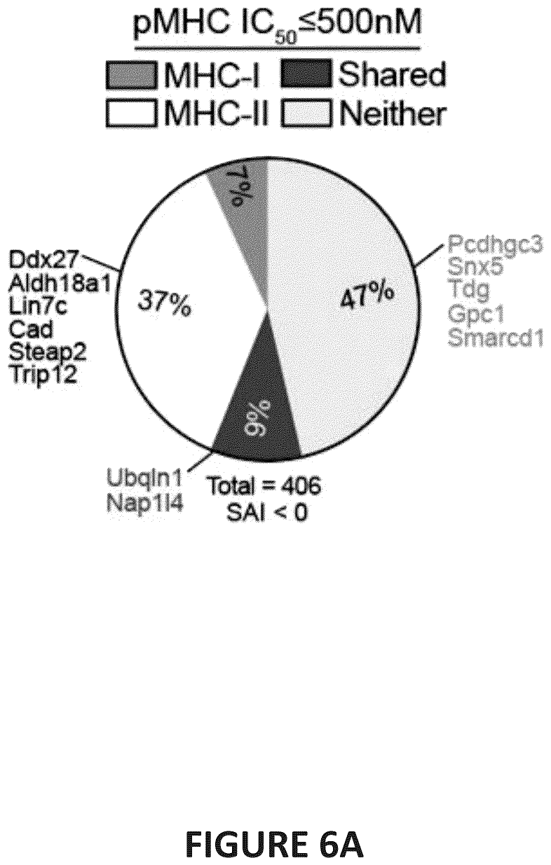

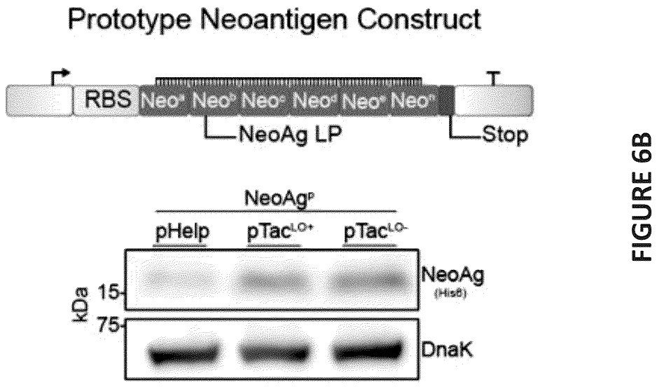

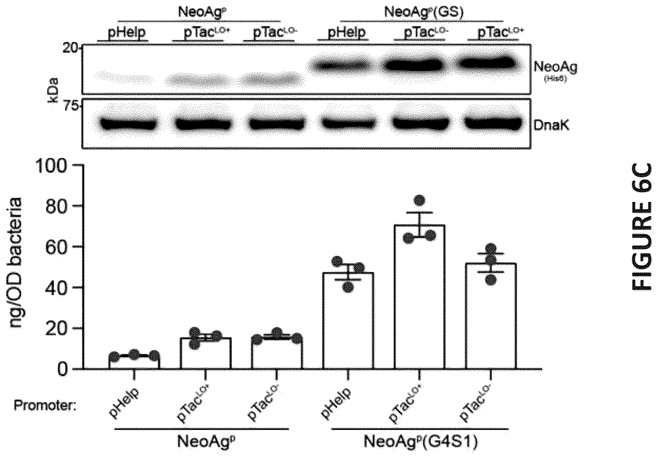

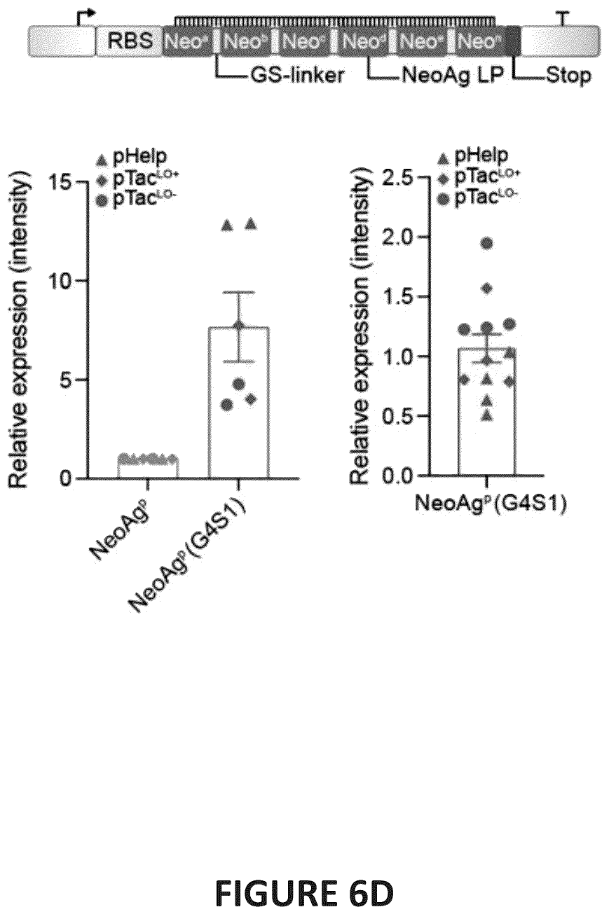

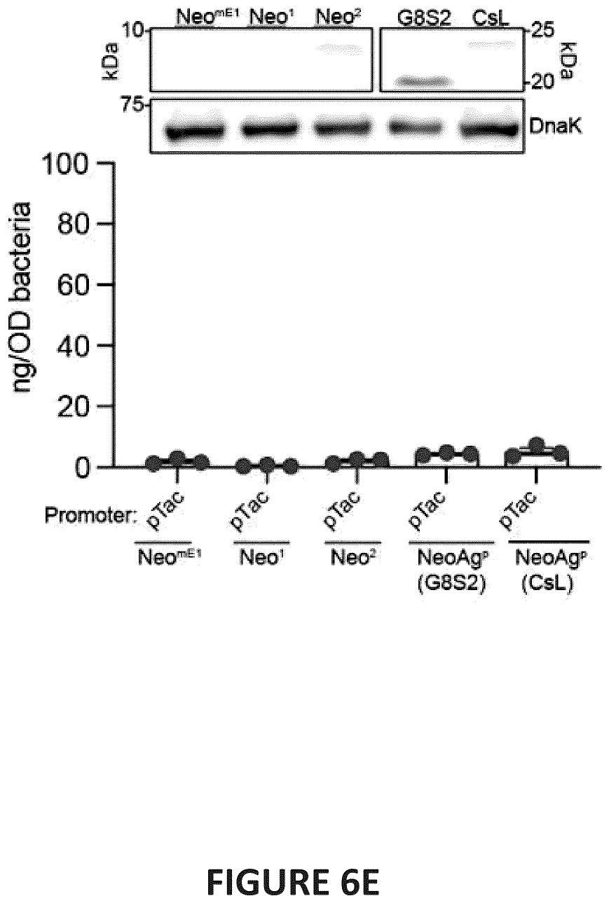

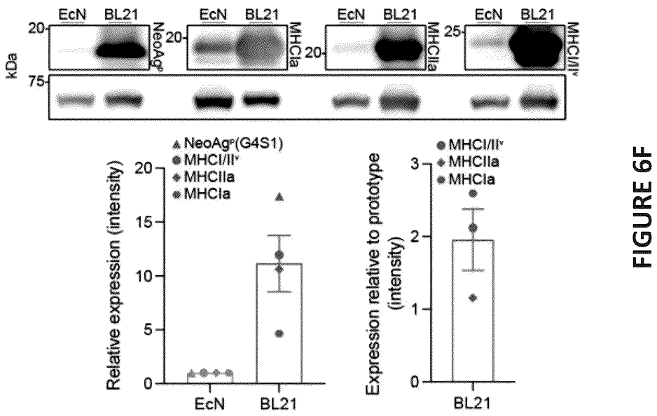

To assemble a repertoire of neoantigens, exome and transcriptome sequencing of subcutaneous CT26 tumors was conducted. Neoantigens were predicted from highly expressed tumor-specific mutations using established methods, with selection criteria inclusive of putative neoantigens across a spectrum of MHC affinity. Given the importance of both MHC class I and MHC class II binding epitopes in anti-tumor immunity, a measure of wildtype-to-mutant MHC affinity ratio—agretopicity or MHC amplitude—was built into both epitope types derived from a given mutation to estimate the ability of adaptive immunity to recognize a neoantigen. Predicted neoantigens were selected from the set of tumor-specific mutations satisfying all criteria, notably encompassing numerous recovered, previously validated CT26 neoantigens (FIG. 6A).

A microbial system that could accommodate the production and delivery of diverse sets of neoantigens to lymphoid tissue and the tumor microenvironment (TME) was subsequently created. For the purpose of assessing neoantigen production capacity, a prototype gene encoding a synthetic neoantigen construct (NeoAg^{p}) was created by concatenating long peptides (LPs) encompassing linked CD4⁺ and CD8⁺ T cell mutant epitopes—previously shown as an optimal form for stimulating cellular immunity—derived from CT26 neoantigens (FIG. 6B, Table 1).

TABLE 1
CT26 peptides in prototype neoantigen construct

| Construct | Gene | AA Mutation | Mutant Sequence in Construct | SEQ ID NO |
|---|---|---|---|---|
| NeoAg^{p} | Aldh18a1 | P154S | LHSGQNHLKEMAISVLEARACAAAGQS | 1 |
| (Prototype) | mULV-gp70 | ERV antigen | LWPKVTYHSPSYVYHQFERKTKYIR | 2 |
| | Ubqln1 | A456V | DTLSAMSNPRAMQVLLQIQQGLQTLAT | 3 |
| | Gpc1 | E165G | YRGANLHLEETLAGFWARLLERLFKQL | 4 |
| | E2f8 | I522T | VILPQAPSGPSYATYLQPAQAQMLTPP | 5 |

| | Mutant MHC-I | SEQ | Predicted MHC-I | MHC-I |
|---|---|---|---|---|
| Construct | Epitope | ID NO | IC₅₀ (nM) | Allele |
| NeoAg^{p} | NHLKEMAISVL | 6 | 4523.3 | H2Kd |
| (Prototype) | SPSYVYHQF | 7 | 98.4 | H2Ld |
| | NPRAMQVLL | 8 | 284 | H2Ld |
| | GFWARLLERL | 9 | 5834.18 | H2Kd |
| | SGPSYATYL | 10 | 36.51 | H2Dd |

| | Mutant MHC-II | SEQ | Predicted MHC-II | MHC-II |
|---|---|---|---|---|
| Construct | Epitope | ID NO | IC₅₀ (nM) | Allele |
| NeoAg^{p} | NHLKEMAISVLEARA | 11 | 53.96 | H2IAd |
| (Prototype) | SYVYHQFERKTKYIR | 12 | 689.64 | H2IEd |
| | MSNPRAMQVLLQIQQ | 13 | 189.26 | H2IAd |
| | LEETLAGFWARLLER | 14 | 556.85 | H2IAd |
| | PSYATYLQPAQAQML | 15 | 82.18 | H2IAd |

The construct was cloned into a stabilized plasmid under constitutive expression and transformed into EcN, however, both immunoblot and ELISA assessment revealed low production of the prototype construct by EcN across several tested promoters (FIG. 6C).

Given the dependency on antigen dosage for establishing an effective and immunodominant antigen-specific immune response, a system for improved recombinant neoantigen construct production was developed. The incorporation of 5-mer glycine-serine (GS) linkers between neoantigen LPs in the prototype increased expression approximately 6-fold (FIGS. 6C and 6D). Conversely, expressing only minimal neoepitopes, decreasing the number of neoantigen LPs in a construct, or incorporating 10-mer GS or immunoprotease-sensitive linkers did not improve production (FIG. 6E).

To evaluate the capacity of constructs with 5-mer GS linkers to accommodate production of various neoantigens, and for eventual in vivo testing, three additional constructs with unique neoantigens from the predicted set were created, selected on a spectrum of predicted affinity for MHC class I and MHC class II (MHCIa, MHCIIa, MHCI/II^{v}) (Table 2).

TABLE 2
CT26 mutant peptides in neoantigen constructs

| Construct | Gene | AA Mutation | Mutant Sequence in Construct | SEQ ID NO |
|---|---|---|---|---|
| MHCIa | Gid8 | P7S | MSYAEKSDEITKDEWMEKLNN | 16 |
| | Glud1 | V546I | NLGLDLRTAAYVNAIEKIFKVYNEAGVTFT | 17 |
| | Mia2 | A317T | KKLIYAAKLNTSLKALEGERN | 18 |
| | Sel1I | A299T | IAHMILGYRYWTGIGVLQSCESALTHYRLV | 19 |
| | C3 | V254I | EPTETFYYIDDPNGLEISIIAKFLYGKNVD | 20 |
| | Hnrnpl | I203R | RSVNSVLLFTILNPIYSRTTDVLYTICNPC | 21 |
| MHCIIa | Birc2 | E395K | EDVVMMSTPVVKAALKMGFSRSLVRQTVQ | 22 |
| | Uvrag | P519L | KRASSENERLQYKTPLPSYNSALTQPGVA | 23 |
| | Aldh18a1 | P154S | LHSGQNHLKEMAISVLEARACAAAGQSGL | 24 |
| | Tnks2 | A1116V | VTLGKSFLQFSAMKMVHSPPGHHSVTGRP | 25 |
| | Tnfaip1 | D50N | SLYYTTVRALTRHNTMLKAMFSGRMEVLT | 26 |
| | Rab3ip | C341R | IEPVGLQPIRFVKASAVERGGPKKCALTG | 27 |
| MHCI/IIV | Mtpap | E341K | MSGFQCDLTANNSIALKSSKLLYIYGSLDS | 28 |
| | Akap9 | V88I | PPYSLEHAKITSETNTQLEHAKITQTELMR | 29 |
| | Gorasp1 | G91D | EVEWPSNMWGDQGLLGASVRFCSFRRAS | 30 |
| | Maged1 | A169G | GPGTTYNFPQSPSGNEMTNNQPKTAKA | 31 |
| | Top1 | T413I | WKEVRHDNKVIWLVSWTENIQGSIKYIML | 32 |
| | Col8a1 | G493D | PKGEPGIPGDQDLQGPPGIPGIVGPSGPI | 33 |
| | Ciao2b | T134A | AIPSPAVQDGCTHYTRDPRLRACSEQTACR | 34 |

| | Mutant MHC-I | SEQ | Predicted MHC-I | MHC-I |
|---|---|---|---|---|
| Construct | Epitope | ID NO | IC₅₀ (nM) | Allele |
| MHCIa | SYAEKSDEI | 35 | 58.46 | H2Kd |
| | AYVNAIEKI | 36 | 34.21 | H2Kd |
| | IYAAKLNTSL | 37 | 90.05 | H2Kd |
| | RYWTGIGVL | 38 | 118.72 | H2Kd |
| | YYIDDPNGLEI | 39 | 69.16 | H2Kd |
| | IYSRTTDVL | 40 | 169.83 | H2Kd |
| MHCIIa | KMGFSRSLV | 41 | 5116.93 | H2Kd |
| | TPLPSYNSAL | 42 | 650.89 | H2Ld |
| | NHLKEMAISV | 43 | 4355.5 | H2Kd |
| | SFLQFSAMKMV | 44 | 3314.24 | H2Kd |
| | RHNTMLKAM | 45 | 381.54 | H2Kd |
| | RGGPKKCALT | 46 | 1067.93 | H2Dd |
| MHCI/IIV | KLLYIYGSL | 47 | 9045.19 | H2Kd |
| | PYSLEHAKI | 48 | 3350.47 | H2Kd |
| | VPSNMWGDQGL | 49 | 454.12 | H2Ld |
| | FPQSPSGNEMT | 50 | 11612.58 | H2Ld |
| | VIWLVSWTENI | 51 | 8233.62 | H2Kd |
| | EPGIPGDQDLQ | 52 | 11943.12 | H2Kd |
| | LRACSEQTA | 53 | 23017.505 | H2Kd |

| | Mutant MHC-II | SEQ | Predicted MHC-II | MHC-II |
|---|---|---|---|---|
| Construct | Epitope | ID NO | IC₅₀ (nM) | Allele |
| MHCIa | EKSDEITKDEWMEKLN | 54 | 16524.89 | H2-IAd |
| | IEKIFKVYNEAGVTF | 55 | 1457.52 | H2-IAd |
| | KKLIYAAKLNTSLKALE | 56 | 93.66 | H2-IAd |
| | YWTGIGVLQSCESAL | 57 | 704.1 | H2-IAd |
| | IDDPNGLEISIIAKF | 58 | 1629.03 | H2-IAd |
| | LFTILNPIYSRTTDV | 59 | 250.11 | H2-IAd |
| MHCIIa | VMMSTPVVKAALKMGFSRS | 60 | 37.7 | H2-IAd |
| | NERLQYKTPLPSYNSALTQ | 61 | 40 | H2-IAd |
| | NHLKEMAISVLEARA | 62 | 48.03 | H2-IAd |
| | GKSFLQFSAMKMVHSPPGH | 63 | 46.2 | H2-IAd |
| | SLYYTTVRALTRHNT | 64 | 58.8 | H2-IAd |
| | EPVGLQPIRFVKASAVERG | 65 | 52.2 | H2-IAd |
| MHCI/IIV | CDLTANNSIALKSSKLLYI | 66 | 114 | H2-IAd |
| | NTQLEHAKITQTELM | 67 | 978.1 | H2-IAd |
| | MWGDQGLLGASVRFCSF | 68 | 1038.2 | H2-IAd |
| | TYNFPQSPSGNEMTNN | 69 | 1091.83 | H2-IAd |
| | DNKVIWLVSWTENIQGSIK | 70 | 1791.8 | H2-IAd |
| | QDLQGPPGIPGIVGPSGPI | 71 | 2775.7 | H2-IAd |
| | RDPRLRACSEQTACR | 72 | 4266.04 | H2-IAd |

Neoantigens were grouped based on high predicted affinity for MHC-I (MHCIa) or MHC-II (MHCIIa), or low-moderate affinity for MHC-I or MHC-II (MHCII^{v}). Prototype and novel construct expression was evaluated in EcN versus BL21, a strain which harbors chromosomal deletions of the Lon (ΔIon) and OmpT (ΔompT) proteases to facilitate recombinant protein production. Unlike BL21, EcN also bears cryptic plasmids which can suppress the copy number of transformed recombinant plasmids. Indeed, on average, BL21 produced 10-fold higher levels of neoantigen construct relative to EcN (FIG. 6F).

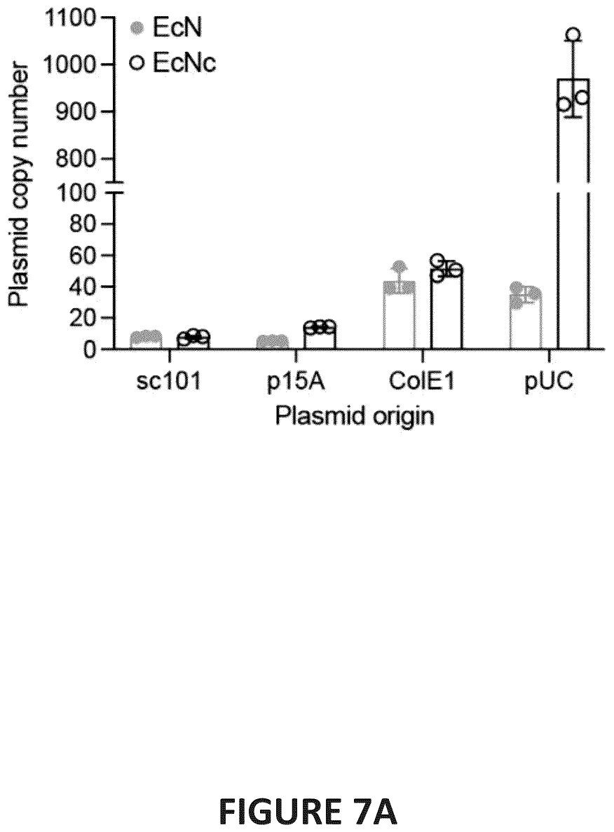

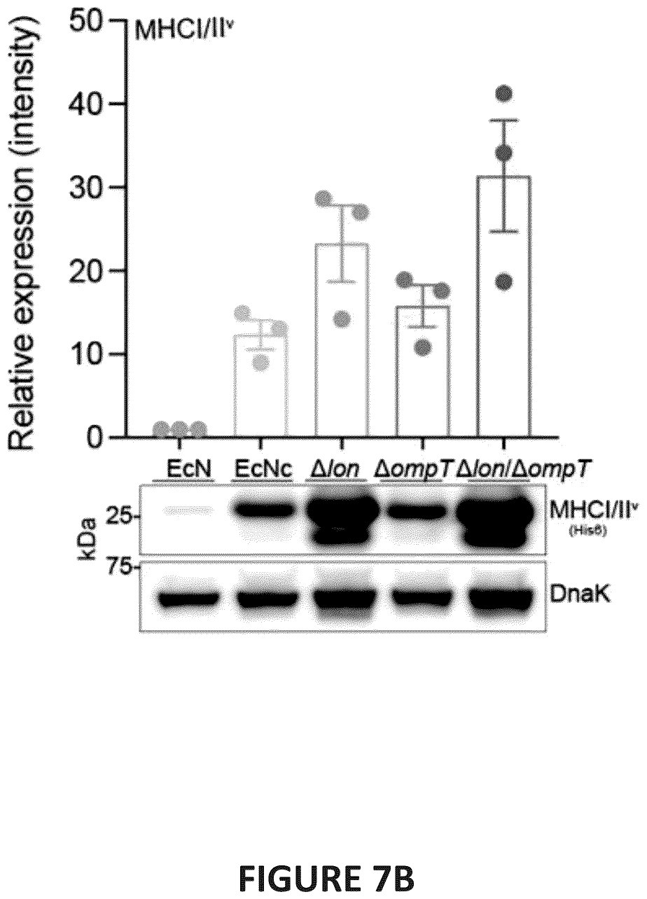

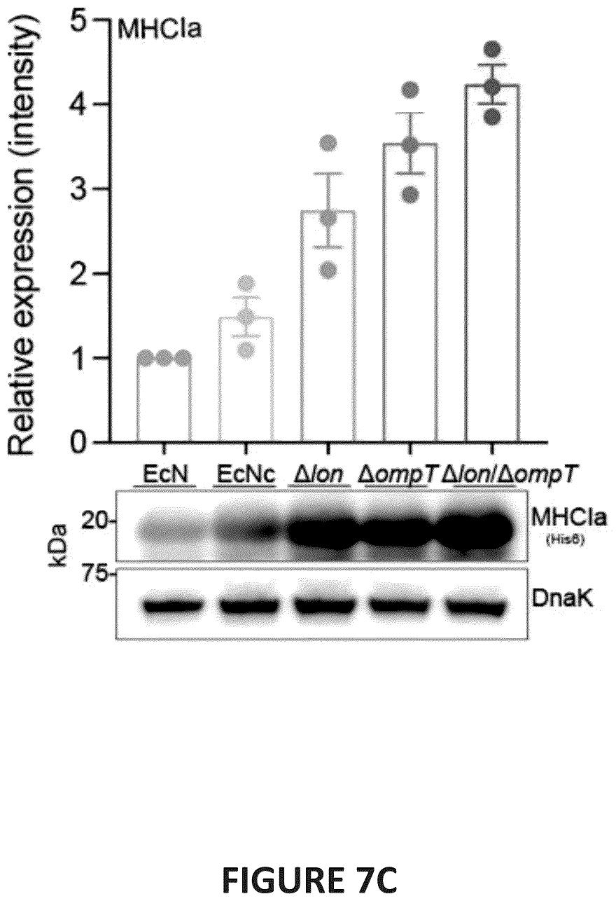

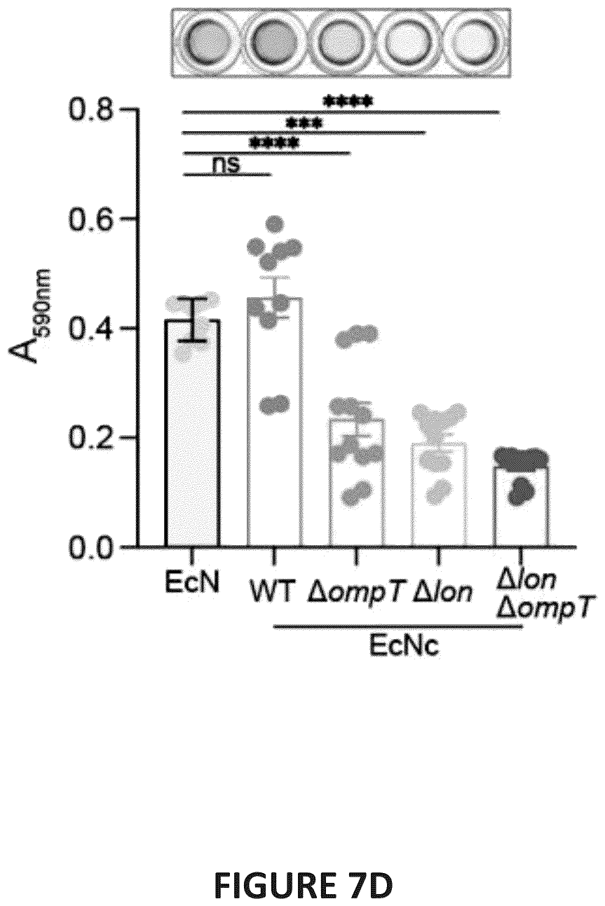

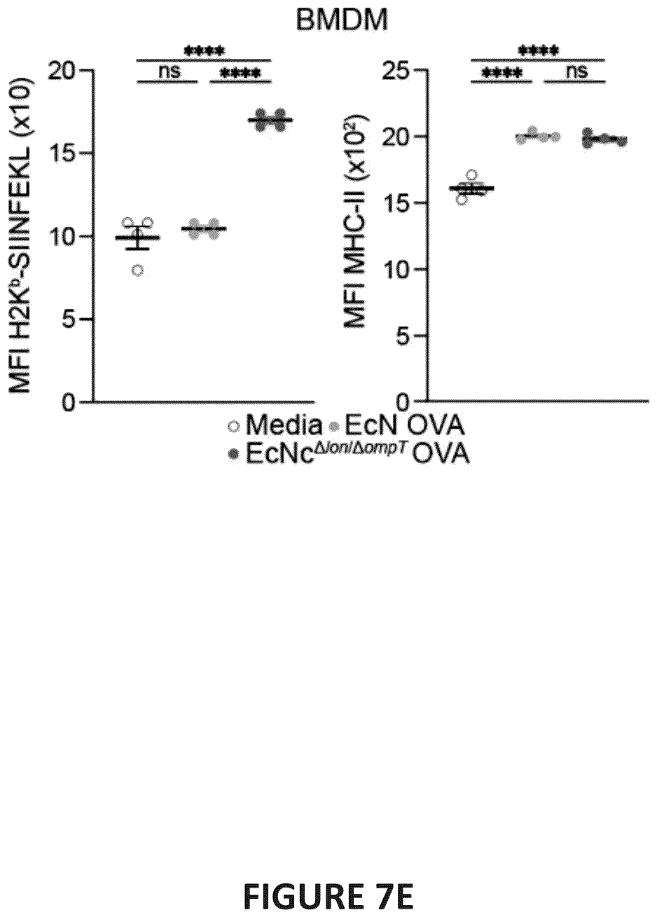

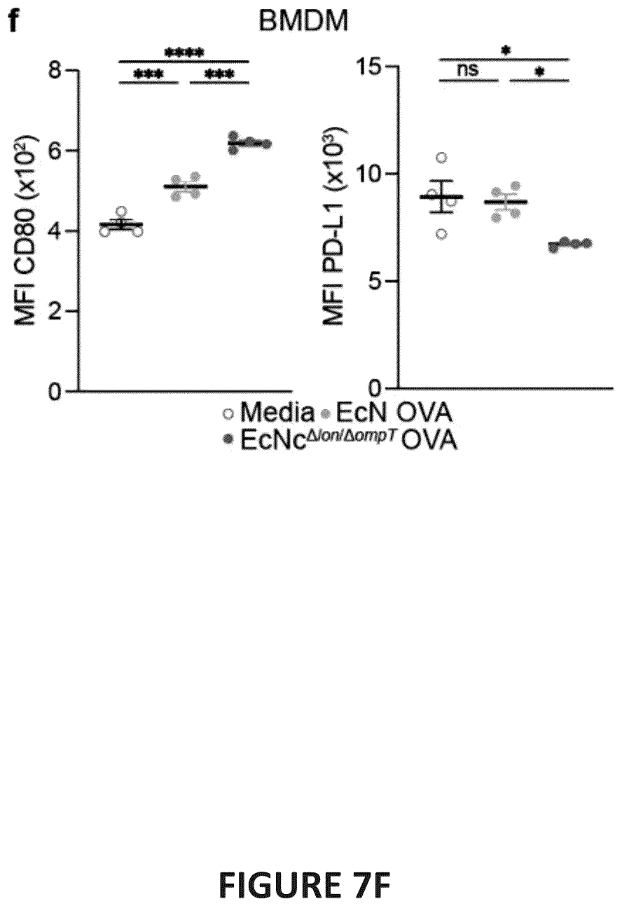

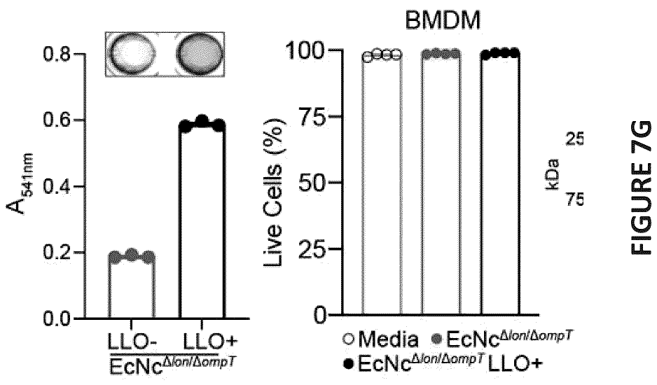

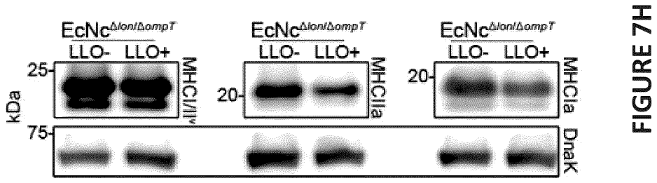

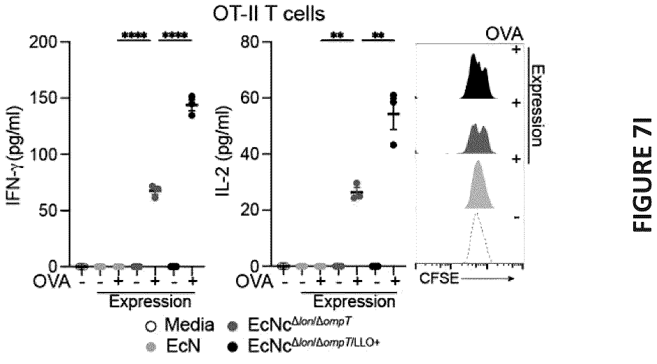

To further enhance construct expression, EcN was synthetically modified. Removal of the EcN cryptic plasmids led to maintenance of approximately 300-fold higher levels of therapeutic plasmid DNA (EcNc) (FIG. 7A), with successive deletion of the Lon protease (EcNc^{ΔIon}), OmpT protease (EcNc^{ΔompT}), or both proteases (EcNc^{ΔIon/ΔompT}) allowing up to 80-fold increased production of synthetic neoantigen constructs relative to the parental EcN strain (FIGS. 1B, 7B, and 7C).

Since the Lon protease has been connected to capsule and biofilm regulation, and OmpT with the degradation of complement, the susceptibility of the engineered vector EcNc^{ΔIon/ΔompT} to phagocytosis and blood clearance and its proficiency in biofilm formation was tested. Notably, EcNc^{ΔIon/ompT} was 4-fold more susceptible to phagocytosis by bone marrow-derived macrophages (BMDMs) relative to EcN (FIG. 1C). Incubation in human blood further revealed a 100-fold greater sensitivity to blood clearance for EcNc^{ΔIon/ΔompT} vs. EcN (FIG. 1D). Moreover, EcNc^{ΔIon/ΔompT} was significantly attenuated in biofilm formation, a major mechanism of microbial resistance to immunity and antimicrobial agents in humans (FIG. 7D). Electron microscopy examination of wildtype EcN, EcNc^{ΔIon}, EcNc^{ΔompT} and EcNc^{ΔIon/ΔompT} revealed altered membrane morphology and progressive reduction of capsular polysaccharides in protease knockout strains, likely facilitating the enhanced sensitivity to blood clearance and phagocytosis (data not shown).

As an anti-tumor vaccine, the microbial platform must additionally facilitate presentation of recombinant antigens and activation of APCs. To evaluate the system in this capacity, the model antigen ovalbumin (OVA) was expressed in the cytosol of EcNc^{ΔIon/ΔompT} using a strategy analogous to that used for synthetic neoantigen constructs. BMDMs pulsed with EcNc^{ΔIon/ΔompT}-OVA, but not EcN-OVA, presented the H2K^{b}-SIINFEKL complex, indicating efficient processing and cross-presentation of recombinant antigens from EcNc^{ΔIon/ΔompT} (FIG. 7E). Furthermore, pulsed BMDMs upregulated MHC class II and CD80, and downregulated PD-L1, demonstrating effective APC activation by EcNc^{ΔIon/ΔompT} expressing a recombinant antigen (FIGS. 7E and 7F).

### Example 24

### Synthetic Neoantigen Construct Optimization

To refine the immune program orchestrated by APCs, it was reasoned that constitutive co-expression of LLO—a pH-dependent protein derived from Listeria which forms pores in the phagolysosomal membrane—would enhance cytosolic delivery of encoded neoantigens for presentation to CD8⁺ T cells, and support APC activation and induction of Th1 immunity. Of note, engineered microbes produced functional LLO, and LLO expression did not affect viability of antigen-presenting cells incubated with LLO-expressing strains (EcNc^{ΔIon/ΔompT/LLO+}) or the co-expression of neoantigen constructs (FIGS. 7G and 7H).

Immunofluorescence microscopy of BMDMs co-incubated with either live EcNc^{ΔIon/ΔompT} OVA or EcNc^{ΔIon/ΔompT/LLO+} OVA confirmed that LLO co-expression enabled recombinant antigen escaping into the cytosol (data not shown). Bone-marrow derived dendritic cells (BMDCs) pulsed with live EcNc^{ΔIon/ΔompT/LLO+} OVA secreted 3-fold higher levels of IL-12p70 as compared to those pulsed with EcNc^{ΔIon/ΔompT} OVA (FIG. 1E), indicating greater Th1 instruction by APCs.

Moreover, BMDCs pulsed with live EcNc^{ΔIon/ΔompT/LLO+} OVA mediated superior activation of naïve OT-I and OT-II T cells, with 2- to 2.5-fold increased secretion of interferon-γ (IFN-γ) and interleukin-2 (IL-2) from both T cell types relative to EcNc^{ΔIon/ΔompT} OVA, and marked proliferation of both OT-I and OT-II T cells. Conversely, BMDCs pulsed with wildtype EcN OVA induced no measurable proliferation of either T cell type, 13- to 15-fold lower secretion of IL-2 and IFN-γ from OT-I T cells, and no detectable cytokine secretion from OT-II T cells. Conversely, BMDCs pulsed with wildtype EcN OVA induced no measurable proliferation of either T cell type, 13- to 15-fold lower secretion of IL-2 and IFN-γ from OT-I T cells, and no detectable cytokine secretion from OT-II T cells (FIGS. 1F and 7I). Taken together, these data suggest recombinant antigens expressed in EcNc^{ΔIon/ΔompT/LLO+} lead to potent antigen-specific activation of both naïve cytotoxic and helper T cells, with incorporation of LLO facilitating both enhanced presentation to CD8⁺ T cells and Th1-type immunity.

Overall, synthetic neoantigen construct optimization and genetic engineering achieved a microbial platform (EcNc^{ΔIon/ΔompT/LLO+}) capable of robust production across diverse sets of tumor neoantigens, which was attenuated in immune-resistance mechanisms, effectively taken up by and proficient in activating antigen-presenting cells, and able to drive potent activation of T cells specific for encoded recombinant antigens to support enhanced cellular immunity.

### Example 25

### Therapeutic Efficacy in Murine Colorectal Carcinoma

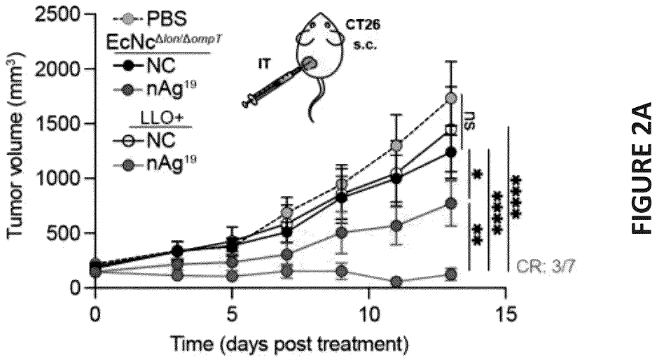

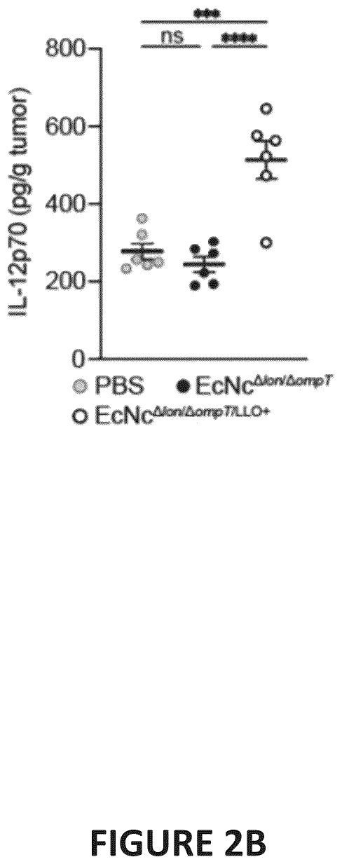

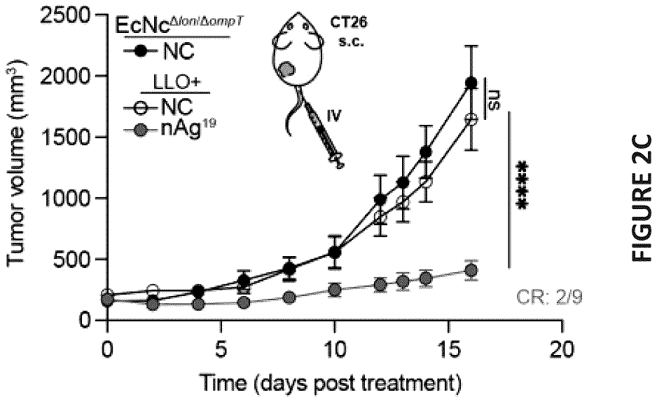

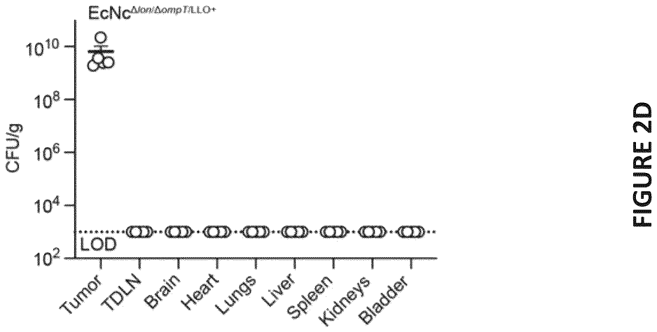

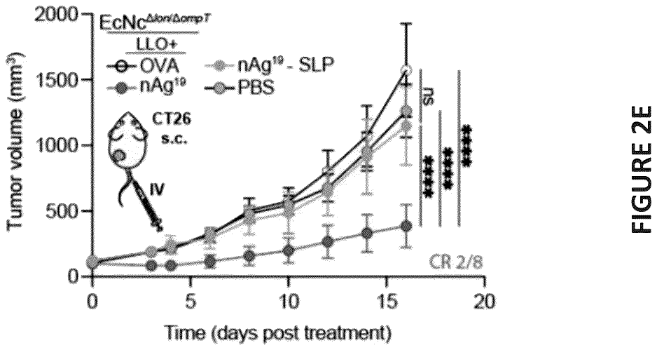

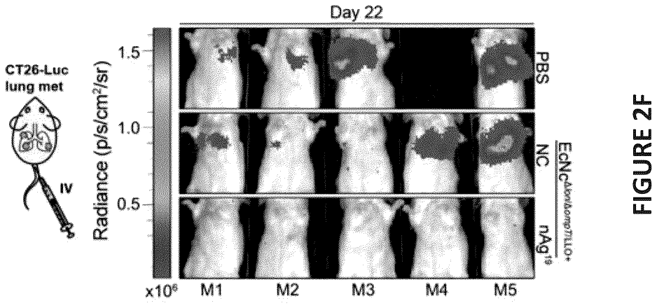

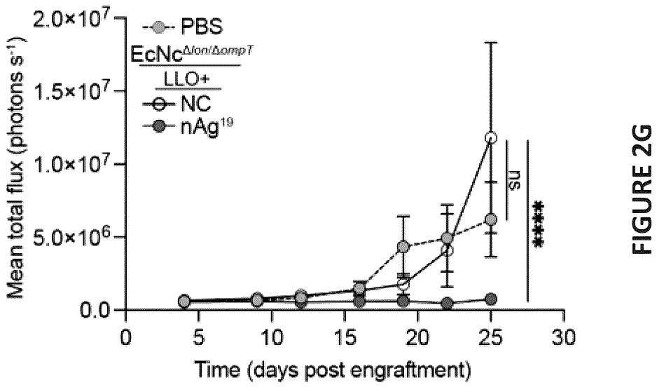

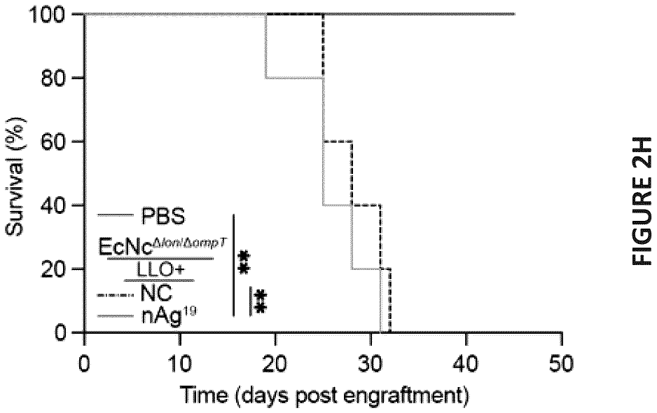

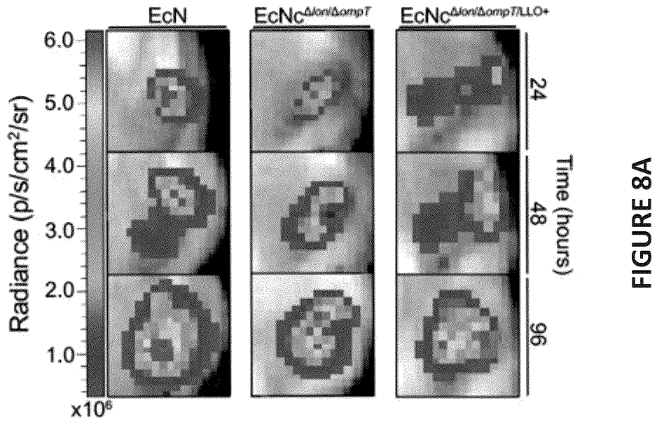

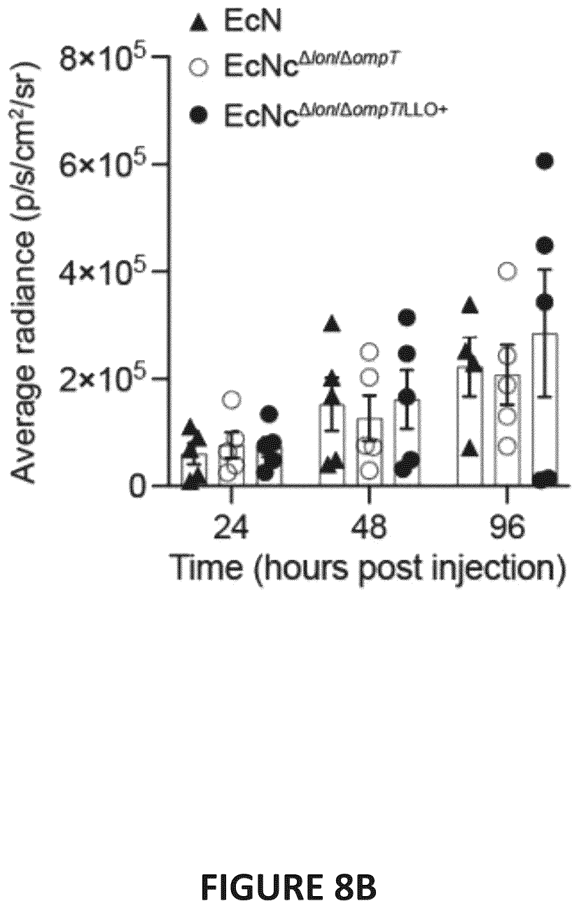

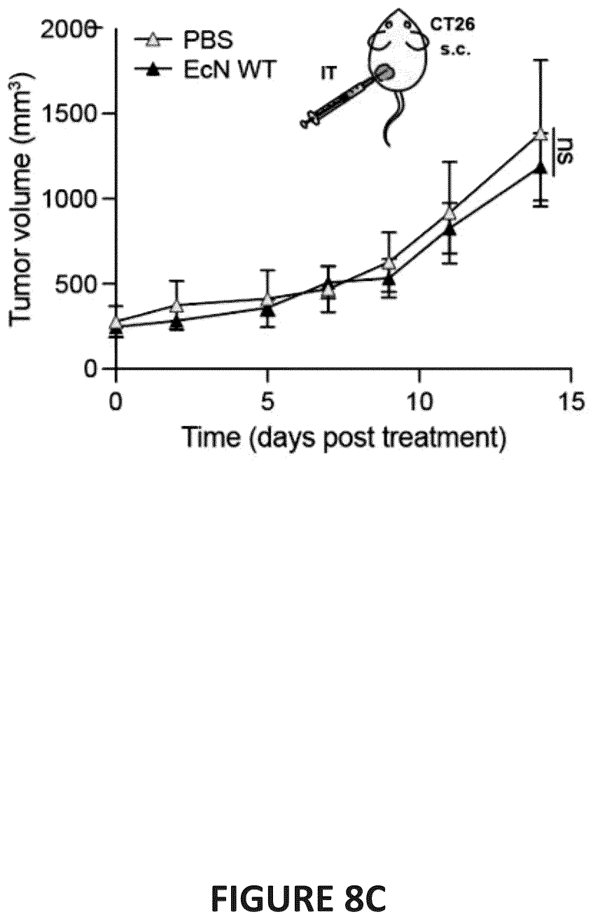

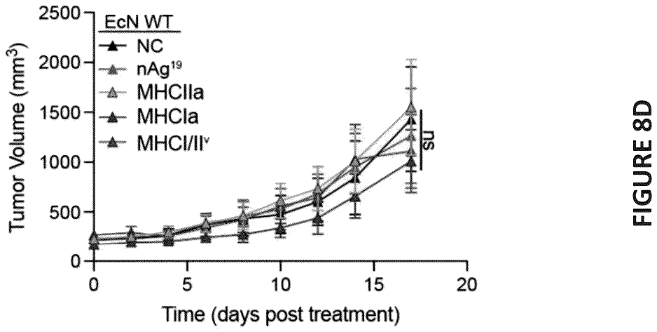

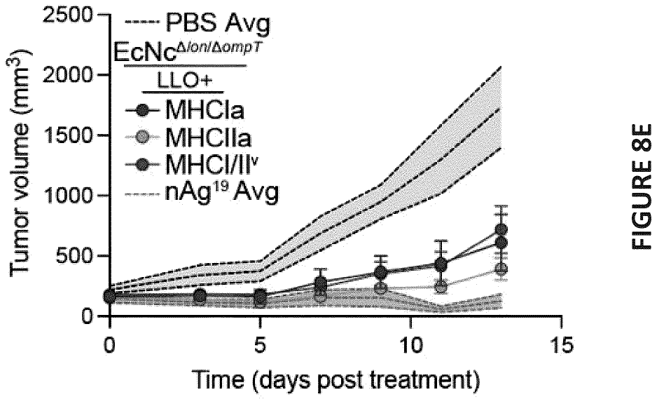

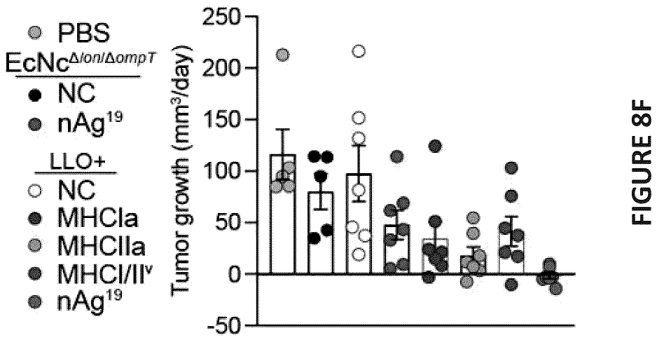

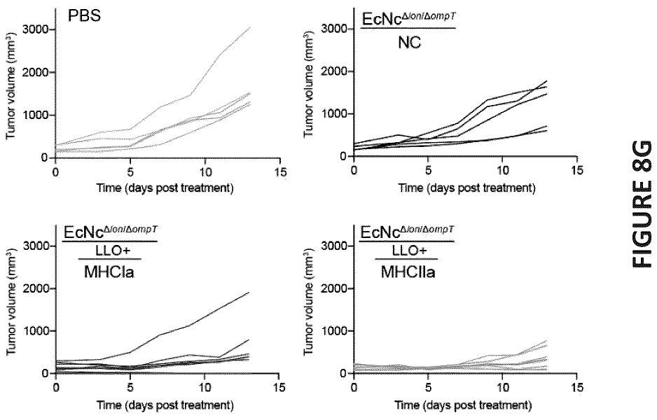

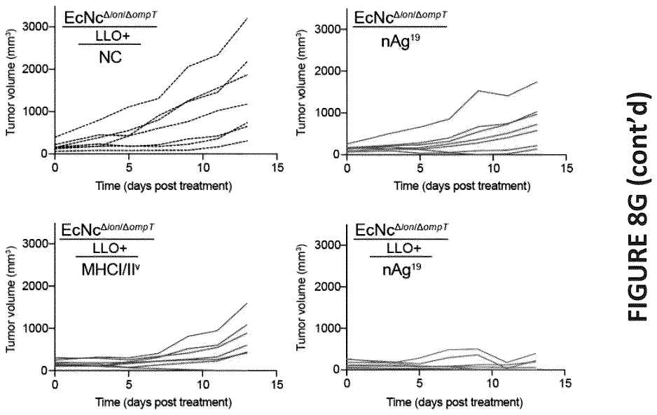

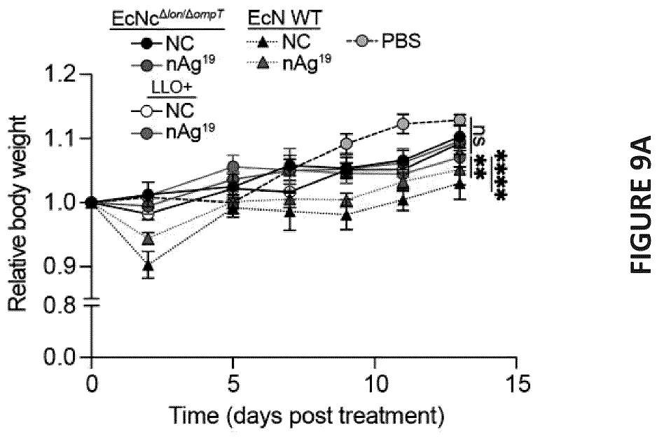

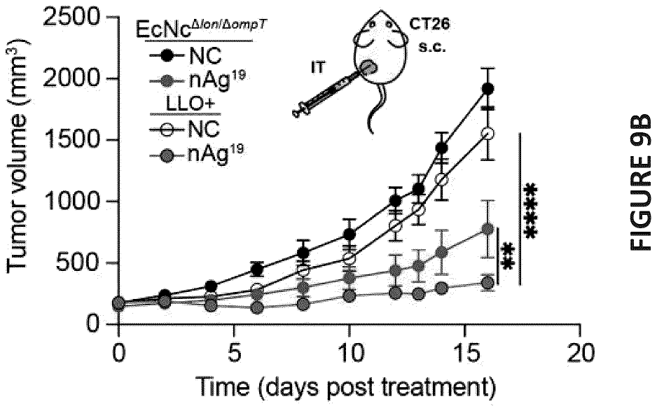

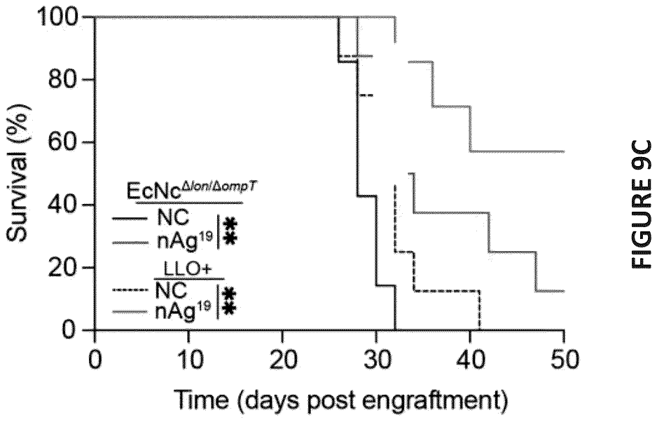

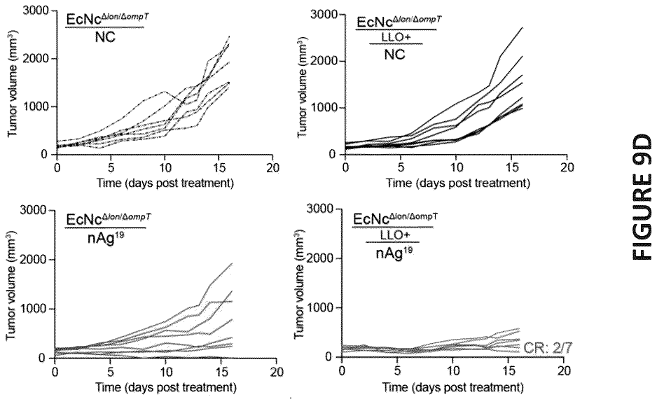

To assess the in vivo efficacy of the engineered system, BALB/c mice bearing advanced CT26 tumors on a single hind-flank received an intratumoral injection of EcN wildtype, EcNc^{ΔIon/ΔompT} or EcNc^{ΔIon/ΔompT/LLO+} strains. These strains were tested either without any neoantigen plasmid (NC), or as a combination of the three neoantigen construct-expressing strains (MHCIa, MHCIIa, and MHCI/II^{v}) in equal parts—a microbial anti-tumor vaccine delivering 19 total unique neoantigens (nAg¹⁹). Notably, no difference in tumor colonization efficiency was observed for EcNc^{ΔIon/ΔompT} strains as compared to wildtype EcN (FIGS. 8A and 8B). Whereas intratumoral treatment with wildtype EcN nAg¹⁹ did not demonstrate any therapeutic benefit (FIGS. 8C and 8D), a single intratumoral injection of EcNc^{ΔIon/ΔompT/LLO+} nAg¹⁹ provided strong anti-tumor efficacy, with a complete response observed for 3 out of 7 tumors and the combination nAg¹⁹ more effective than any construct alone (FIGS. 2A and 8E-8G). Moreover, treatment with EcNc^{ΔIon/ΔompT} and EcNc^{ΔIon/ΔompT/LLO+} strains was well-tolerated, with significantly attenuated body weight change as compared to wildtype EcN, and no significant body weight differences as compared to PBS treatment over the course of observation (FIG. 9A). Direct comparisons of intratumoral treatment with EcNc^{ΔIon/ΔompT/LLO+} nAg¹⁹ vs. EcNc^{ΔIon/ΔompT} nAg¹⁹ revealed that the inclusion of LLO significantly enhanced tumor control and extended survival (FIGS. 2A and 9B-9D). Assessment of intratumoral IL-12p70 levels demonstrated that inclusion of LLO also significantly enhanced IL-12p70 levels in tumors, suggestive of enhanced Th1 instruction in vivo (FIG. 2b). Thus, the combination of all synthetic modifications (EcNc^{ΔIon/ΔompT/LLO+} nAg¹⁹) synergized to produce a microbial anti-tumor vaccine with favorable toxicity profile and strong therapeutic effect in vivo.

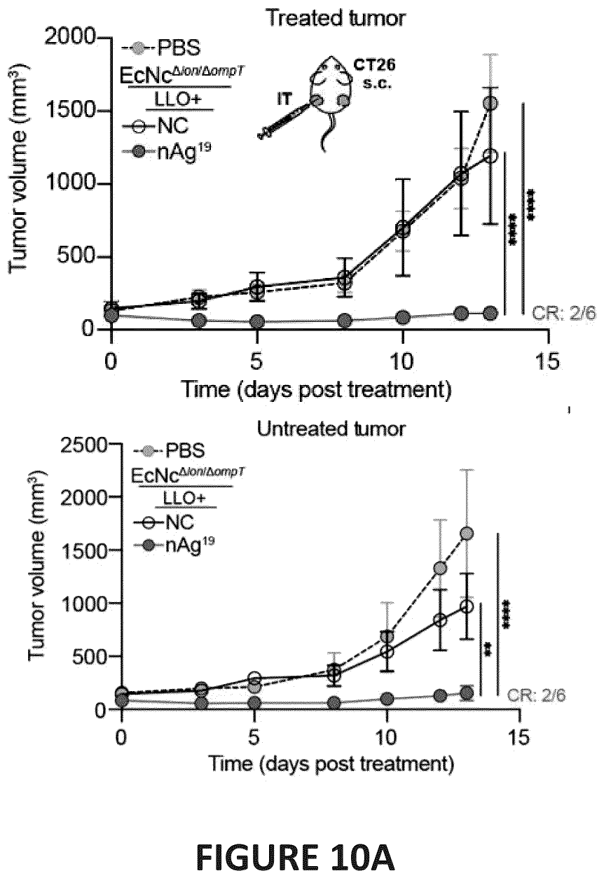

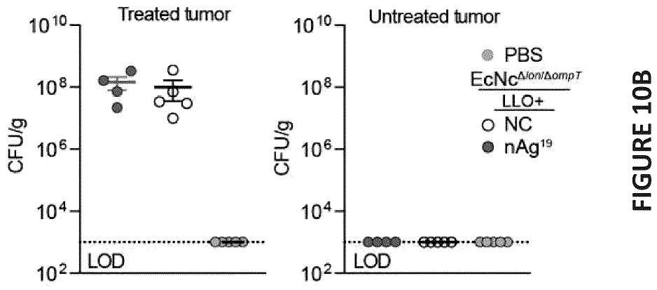

To evaluate the induction of systemic anti-tumor immunity after treatment with the microbial neoantigen vaccines, mice with established CT26 tumors on both hind-flanks were treated with an injection of microbes into a single tumor. Whereas treatment with EcNc^{ΔIon/ΔompT/LLO+} without neoantigen expression (NC) did not suppress tumor growth, a single injection of EcNc^{ΔIon/ΔompT/LLO+} nAg¹⁹ induced tumor control and complete regression of 2 of 6 treated and untreated tumors (FIG. 10A). Microbial quantification from tumors 14-days after injection revealed that microbes colonized treated tumors at high densities, with no bacteria able to be cultured from untreated tumors (FIG. 10B). This demonstrates that the engineered neoantigen vaccines stimulate systemic anti-tumor immunity capable of eliminating distant tumors.

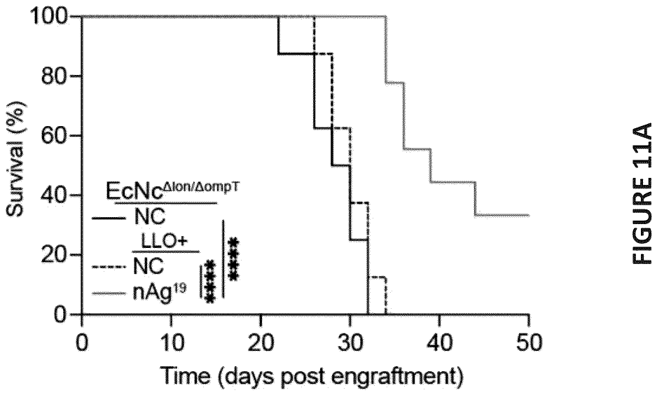

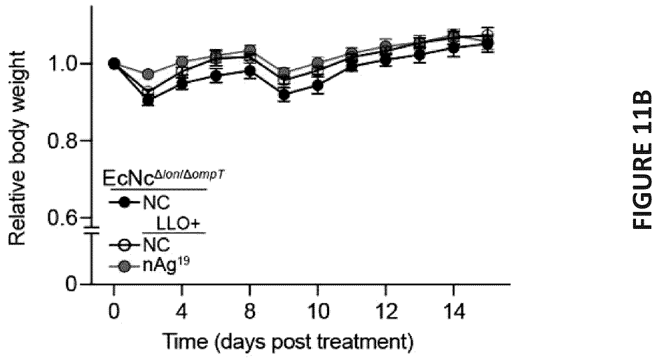

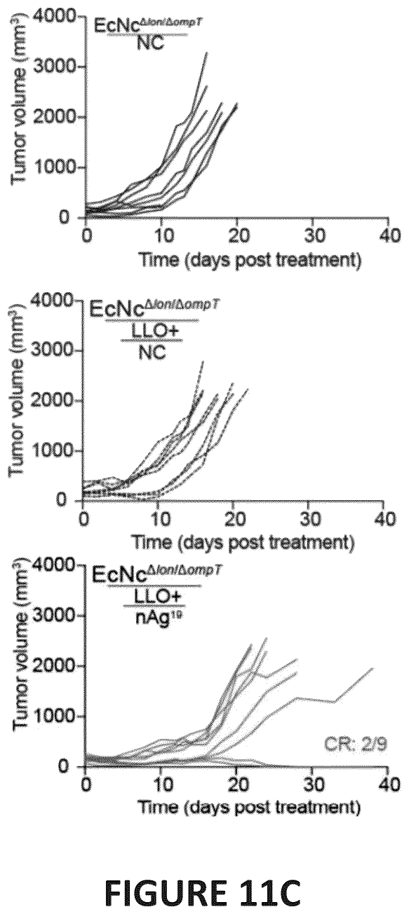

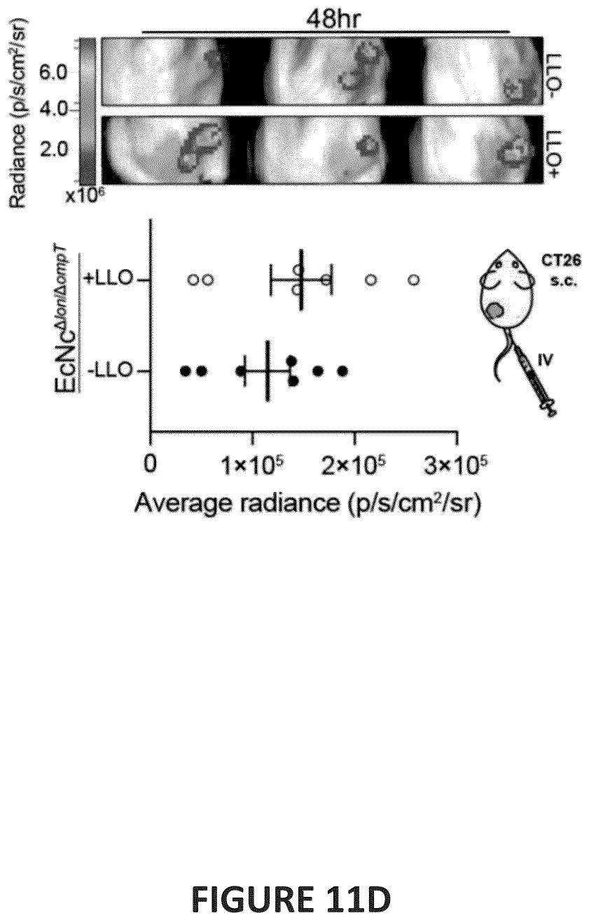

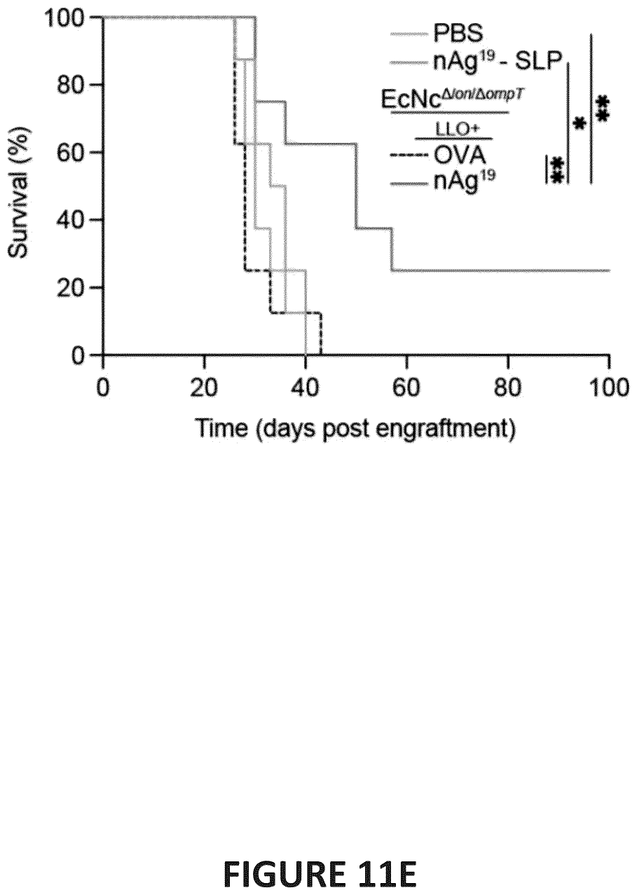

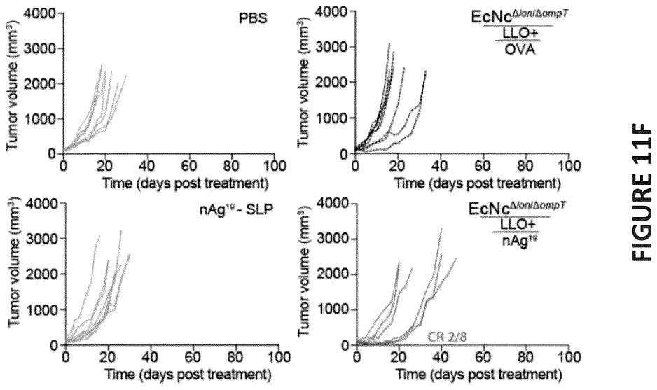

The efficacy of the microbial anti-tumor vaccination platform following intravenous administration was subsequently evaluated. Similar to intratumoral treatment, intravenous administration of EcNc^{ΔIon/ΔompT/LLO+} nAg¹⁹ to mice with advanced CT26 tumors again provided potent anti-tumor efficacy and survival benefit with minimal body weight alteration (FIGS. 2C and 11A-11C). After intravenous injection, the engineered microbes persisted at high density within tumors and were cleared rapidly from all other surveyed organs (FIGS. 2D and 11D).

Given the potent efficacy observed, intravenous treatment was compared to a standard anti-tumor vaccination strategy utilizing synthetic long peptide (SLP) vaccination. Mice bearing established hind-flank CT26 tumors received intravenous injections of either PBS, EcNc^{ΔIon/ΔompT/LLO+} expressing the strong irrelevant xenoantigen OVA, or EcNc^{ΔIon/ΔompT/LLO+} nAg19, or subcutaneous injections of SLP vaccination containing the 19 neoantigens (nAg19-SLP). Compared to SLP vaccination, treatment with EcNc^{ΔIon/ΔompT/LLO+} nAg19 significantly reduced tumor growth and extended survival, with complete regression of 2 of 8 tumors in the EcNc^{ΔIon/ΔompT/LLO+} nAg19 treated group (FIGS. 2E, 11E, and 11F).

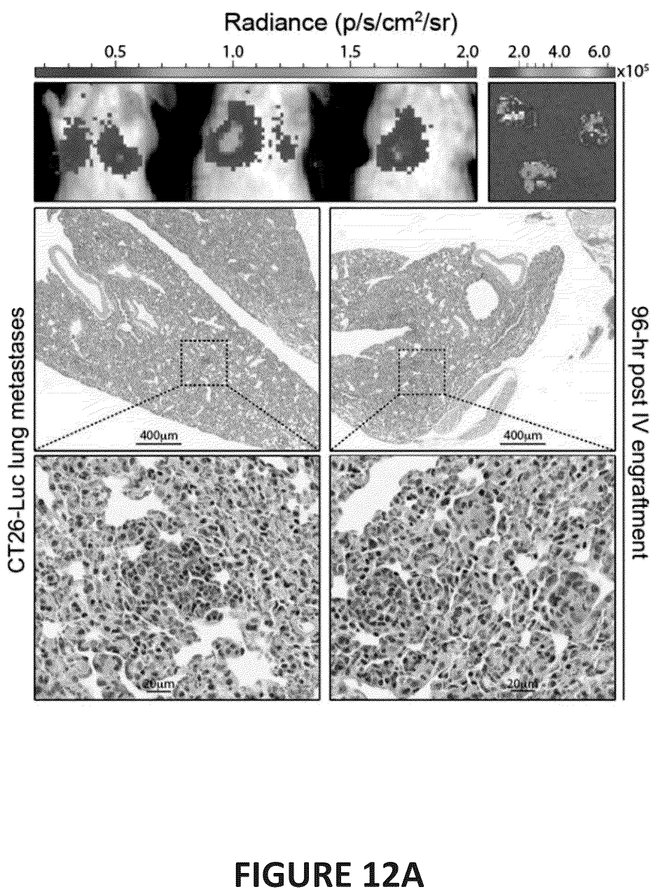

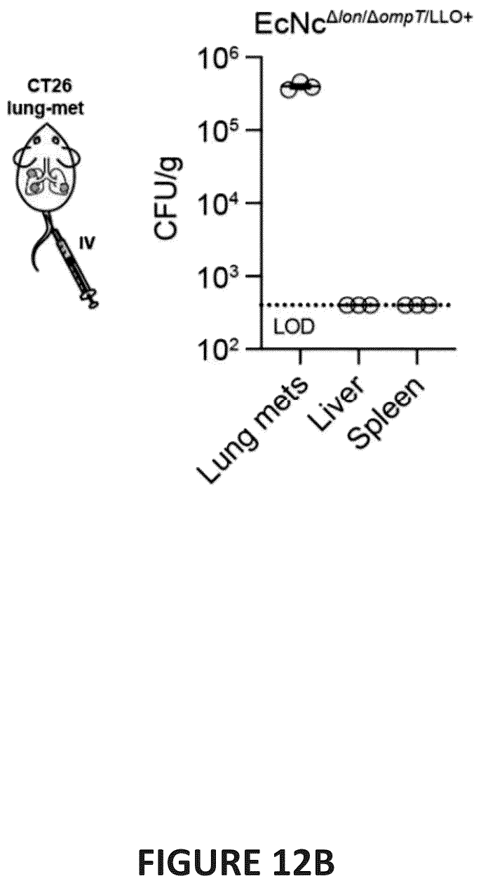

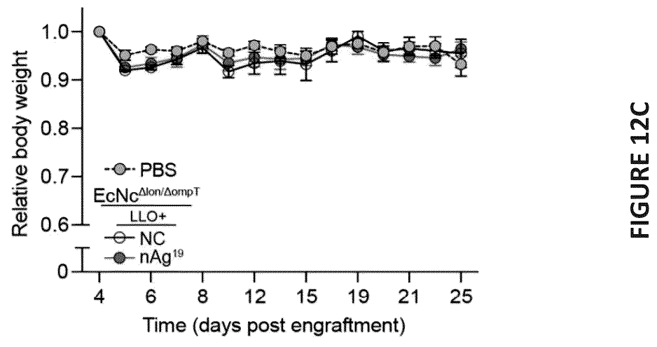

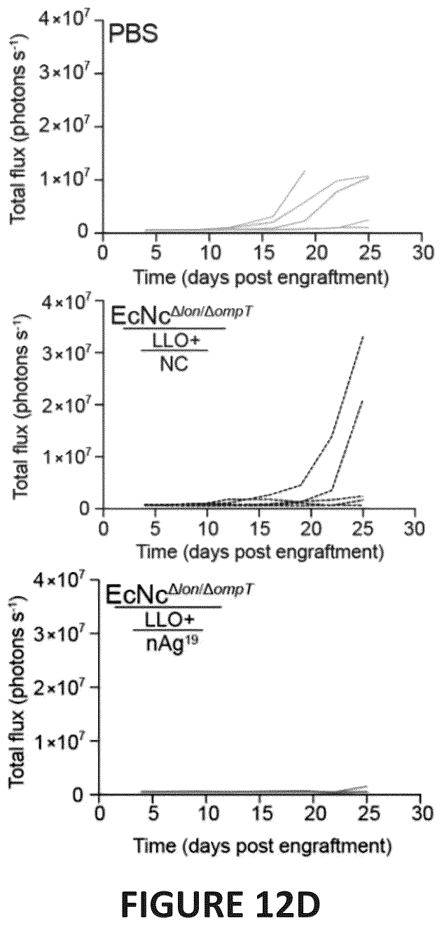

Having observed robust efficacy via intravenous delivery, therapeutic efficacy against established metastatic disease was then evaluated (FIG. 12A). CT26 carcinoma cells with genomically-integrated firefly luciferase (CT26-Luc) were injected intravenously, which form rapidly progressive lung-metastases traceable by bioluminescence imaging (FIG. 12A). Intravenous treatment with PBS, EcNc^{ΔIon/ΔompT/LLO+} without neoantigen expression (NC), or EcNc^{ΔIon/ΔompT/LLO+} nAg¹⁹ was initiated 4-days after engraftment. Engineered microbes colonized metastases-bearing lungs and were not detectable in other tissues (FIG. 12B). Microbial treated groups again demonstrated minimal body weight fluctuation, similarly to mice treated with PBS (FIG. 12C). Treatment with EcNc^{ΔIon/ΔompT/LLO+} nAg¹⁹ strongly restrained metastatic growth, with 100% of neoantigen therapeutic-treated mice surviving to 45 days after engraftment, whereas both control groups had completely succumbed to disease (FIGS. 2F-2H and 12D). These data demonstrate both safety and efficacy of intravenously administered EcNc^{ΔIon/ΔompT/LLO+} nAg¹⁹ in the setting of aggressive, established metastatic disease.

### Example 26

### Dynamics of Microbial-Stimulated Immunity in Colorectal Carcinoma

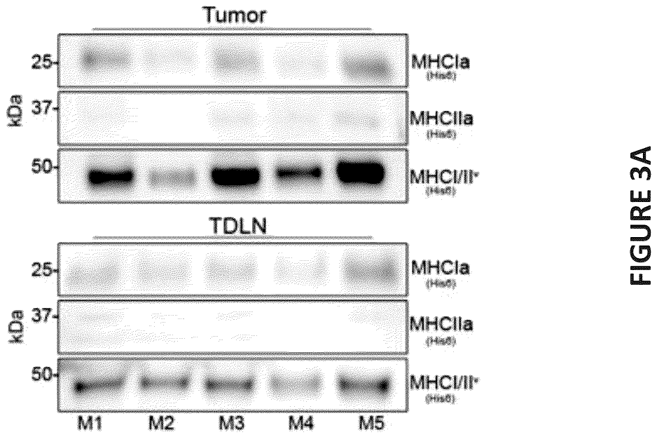

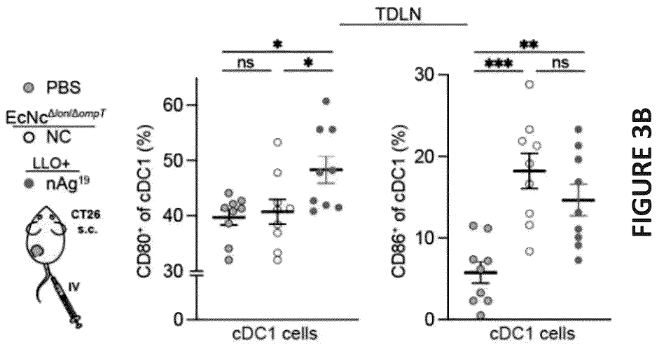

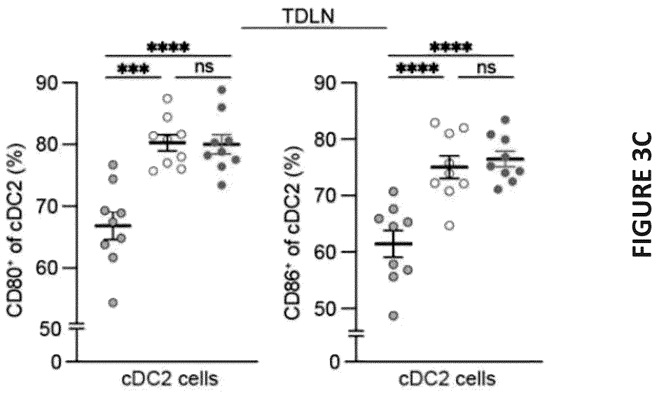

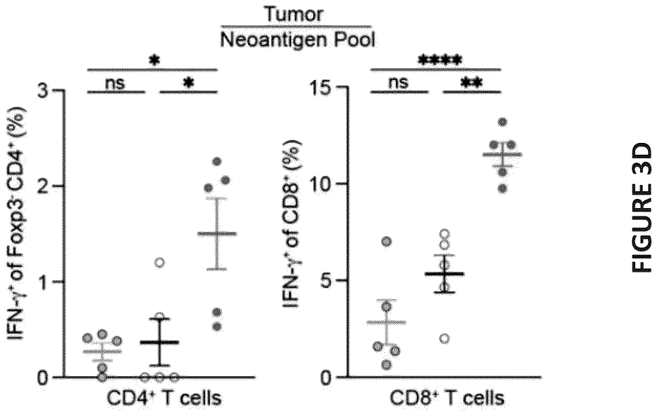

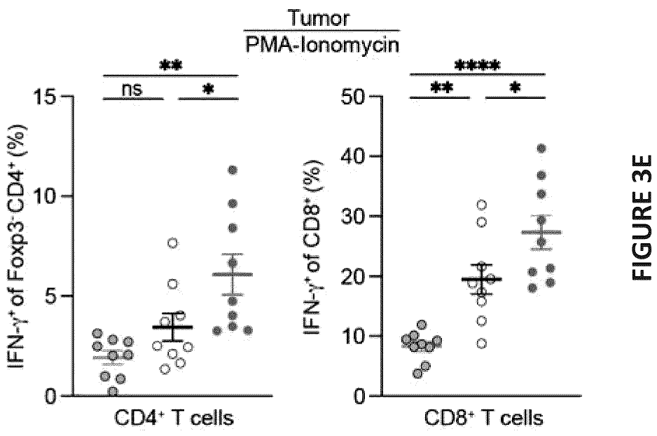

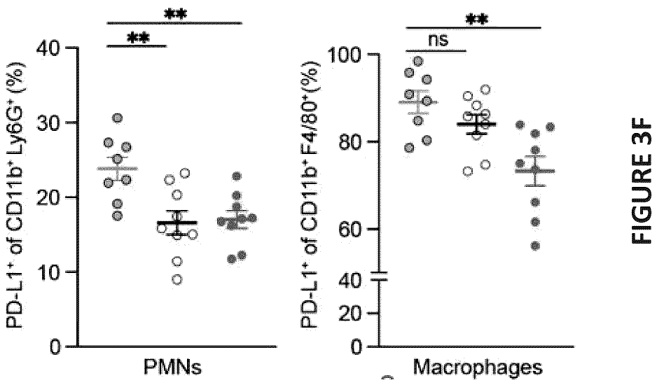

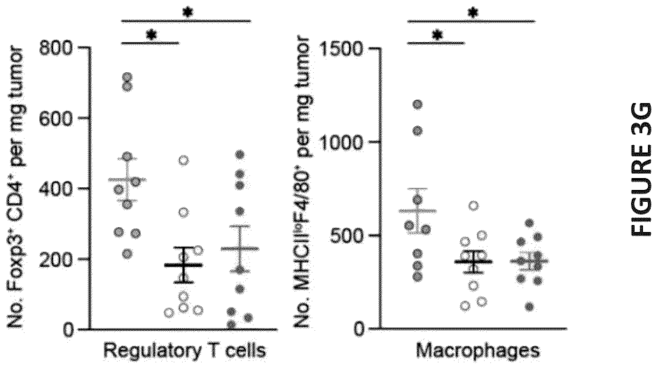

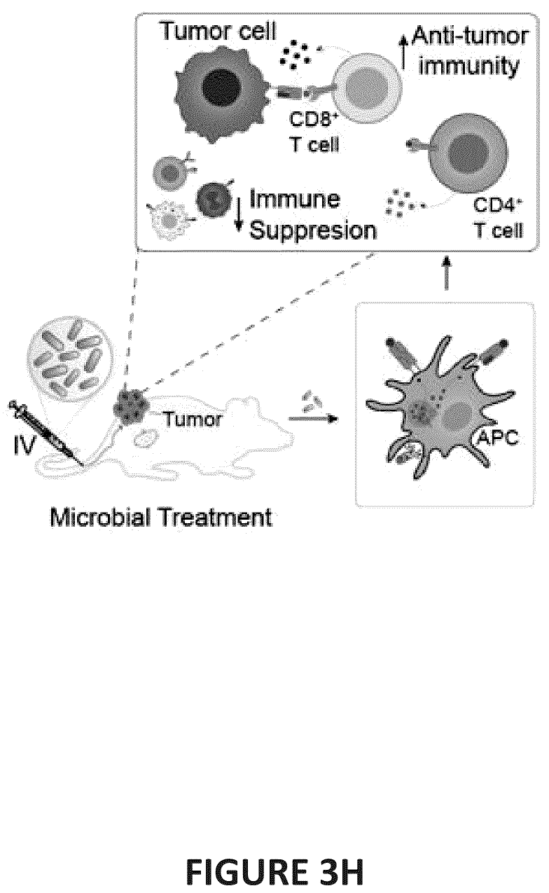

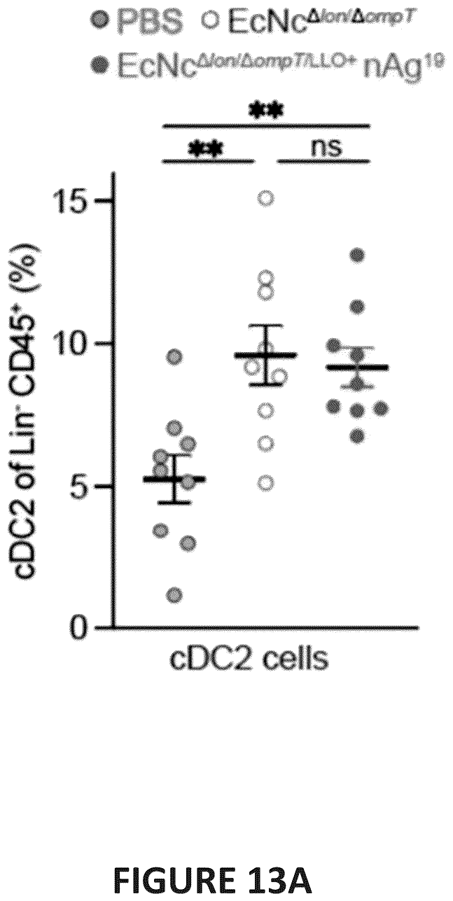

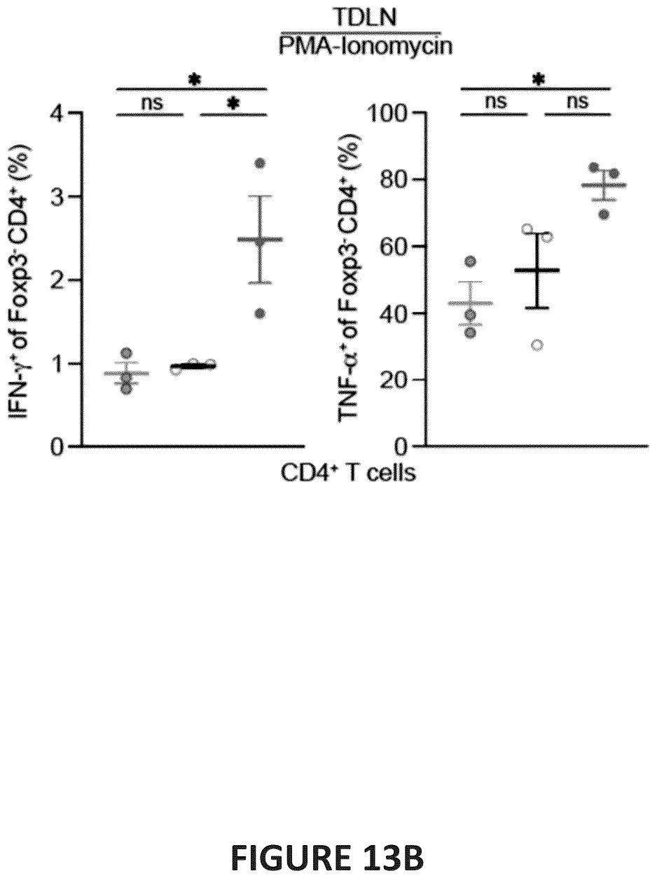

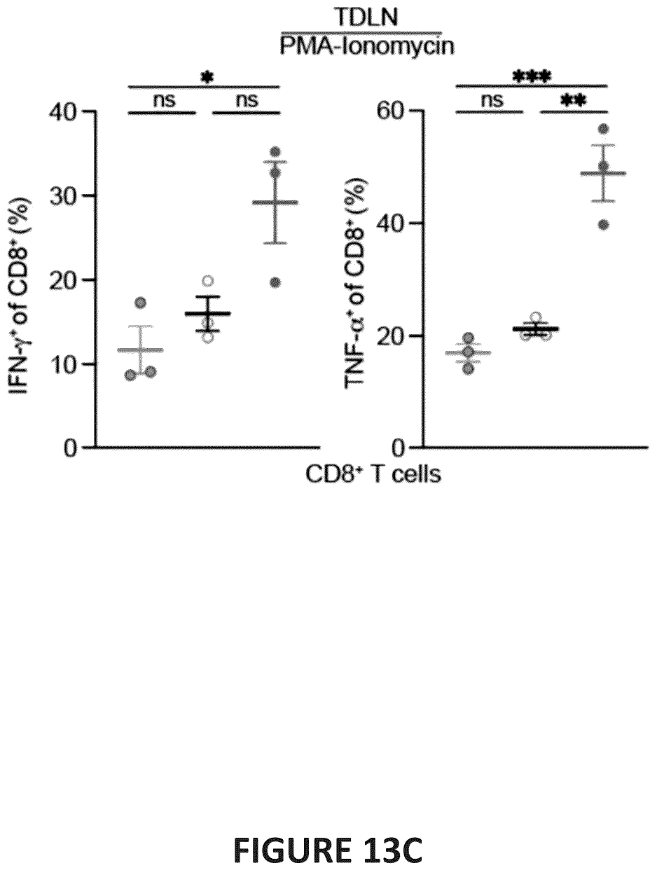

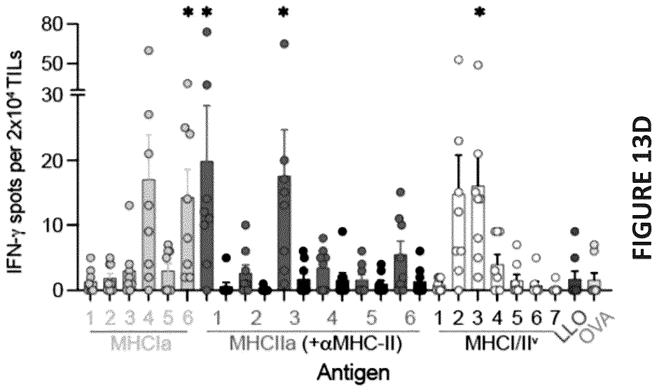

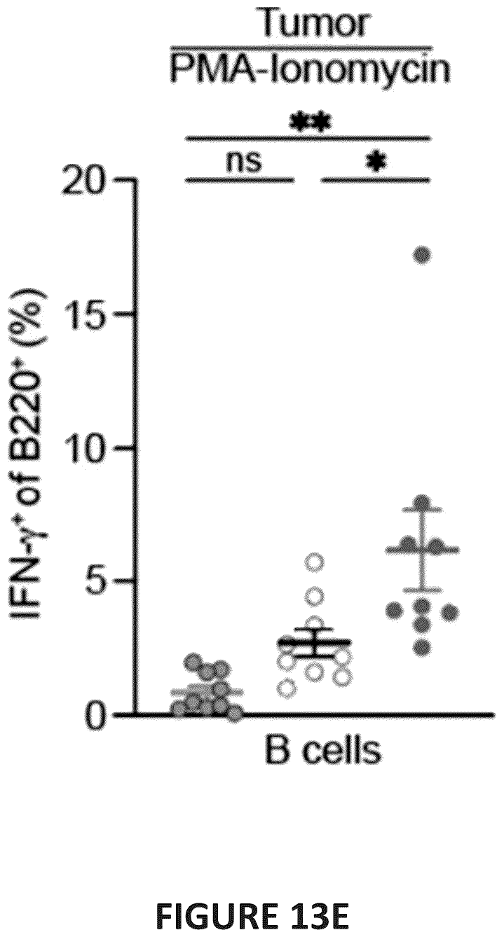

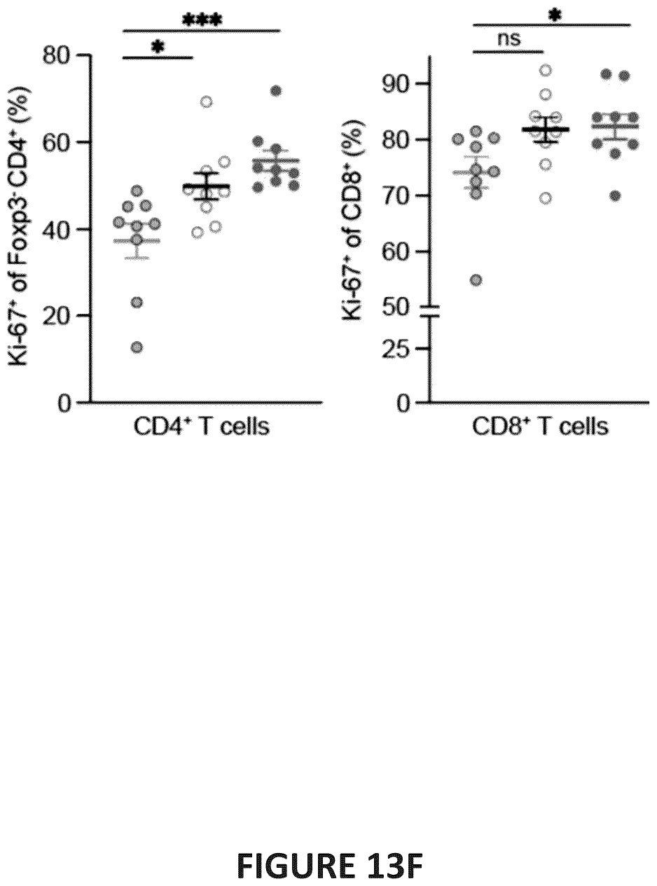

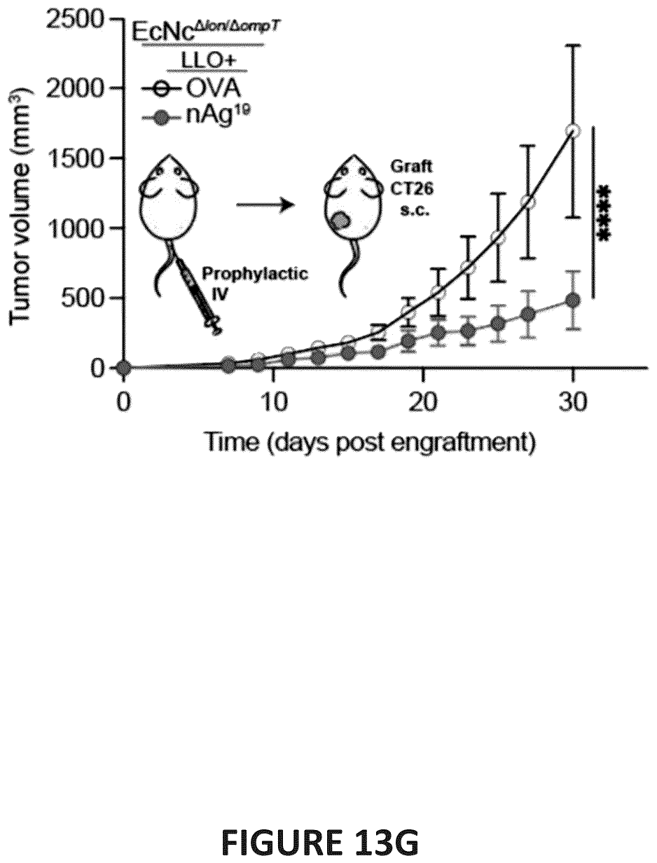

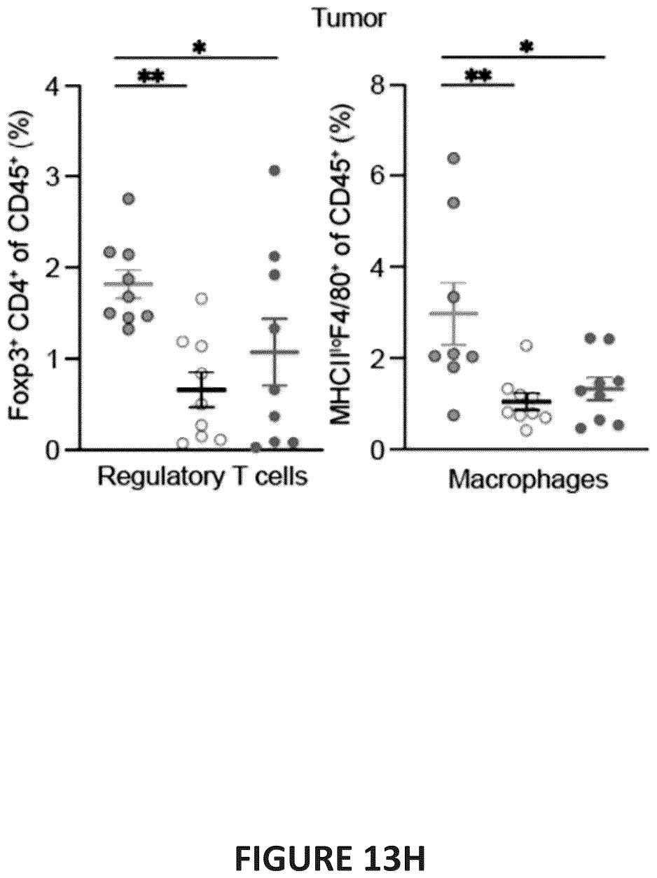

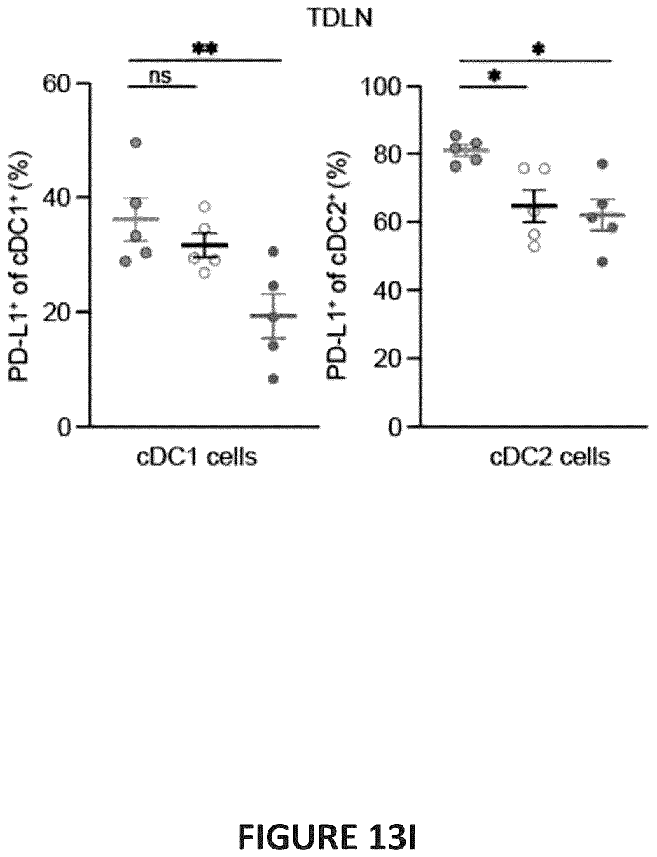

As the engineered microbial neoantigen vaccines are strong immunostimulants and persist within the TME, it was reasoned that sustained intratumoral neoantigen production and reduced immunosuppression would facilitate enhanced activation of adaptive immunity to mediate the observed tumor control. To confirm in situ delivery of encoded neoantigens, EcNc^{ΔIon/ΔompT/LLO+} nAg^{19-His} (wherein all three neoantigen constructs contain a C-terminal 6×His-tag) were intravenously administered and immunoblots of tumor and tumor draining lymph node (TDLN) lysates 2 days following treatment were performed. Three His-tagged protein species —corresponding to the 3 encoded neoantigen constructs—were observed (FIG. 3A). Immunophenotyping of TDLNs 2 days following intravenous treatment revealed significantly higher frequencies of cDC2 cells in TDLNs of microbial vector treated mice (FIG. 13A). Enhanced frequencies of both CD80⁺ and CD86⁺ cDC1 and cDC2 cells were also observed (FIGS. 3B, 3C), demonstrating that intravenously delivered microbial vectors recruit and activate dendritic cells within the TDLN. Consistent with delivered neoantigens enhancing T cell activation, ex vivo restimulation of lymphocytes isolated from TDLNs at 8 days post-treatment with phorbol myristate acetate (PMA) and ionomycin revealed increased production of IFN-γ and TNF-α by conventional CD4⁺ (Foxp3⁻CD4⁺) and CD8⁺ T cells in mice treated with EcNc^{ΔIon/ΔompT/LLO+} nAg¹⁹ vs. those treated with PBS or control bacteria (EcNc^{ΔIon/ΔompT}) (FIGS. 13B, 13C).

Next, to assess the ability for engineered neoantigen therapeutics to drive neoantigen-specific immunity, tumor-infiltrating lymphocytes (TILs) were isolated at 8 days post-treatment and restimulated ex vivo with a pool of synthetic peptides representing the 19 bacterially-encoded tumor neoantigens. Flow cytometric analysis revealed increased frequencies of IFN-γ secreting conventional Foxp3⁻CD4⁺ and CD8⁺ TILs, demonstrating that treatment with EcNc^{ΔIon/ΔompT/LLO+} nAg¹⁹ enhanced encoded neoantigen-specific immunity (FIG. 3D). Analysis of TIL reactivity ex vivo demonstrated that multiple predicted MHC-I and MHC-II binding neoantigens from each neoantigen construct were targeted in EcNc^{ΔIon/ΔompT/LLO+} nAg¹⁹ treated mice (FIG. 13D). Compared to peptide stimulation, restimulation with PMA and ionomycin revealed even greater levels of IFN-γ secreting Foxp3⁻CD4⁺ and CD8⁺ TILs in EcNc^{ΔIon/ΔompT/LLO+} nAg¹⁹-treated tumors, and IFN-γ producing B220⁺ B-cells, suggestive of epitope spreading and expanded immune activation (FIGS. 3E and 13E). Additionally, increased frequencies of proliferating CD4⁺ and CD8⁺ tumor-infiltrating T cells were observed in mice treated with EcNc^{ΔIon/ΔompT/LLO+} nAg¹⁹ in comparison to treatment with EcNc^{ΔIon/ΔompT} or PBS (FIG. 13F). To establish whether microbial neoantigen vaccine treatment generates tumor-neoantigen specific immune memory, naïve mice were prophylactically vaccinated with EcNc^{ΔIon/ΔompT/LLO+} OVA or nAg19 and grafted CT26 tumors post vaccination. Tumor growth in mice prophylactically treated with EcNc^{ΔIon/ΔompT/LLO+} nAg¹⁹ was significantly reduced as compared to those treated with EcNc^{ΔIon/ΔompT/LLO+} OVA, indicating generation of tumor-neoantigen specific immune memory (FIG. 13G).

Beyond induction of tumor antigen-specific T cell responses, treatment with EcNc^{ΔIon/ΔompT/LLO+} nAg¹⁹ resulted in reduced frequencies of tumor-resident immunosuppressive PD-L1⁺ Ly6G⁺ polymorphonuclear cells (PMNs) and PD-L1⁺ F4/80⁺ macrophages (FIG. 3F). Bacteria-treated groups further displayed reduced numbers and frequencies of Foxp3⁺CD4⁺ regulatory T cells and MHC-II^{lo}F4/80⁺ tumor-associated macrophages (FIGS. 3G and 13H), two cell populations known for their roles in inhibiting anti-tumor immunity. Moreover, myeloid immunophenotyping revealed a reduction of PD-L1 on cDC1 and cDC2 populations within TDLNs of the neoantigen therapeutic treated group (FIG. 13I), which has been shown to facilitate and sustain anti-tumor immunity. In summary, intravenously delivered microbial neoantigen therapeutics sustain neoantigen production and availability in lymphoid tissue in vivo, stimulate both neoantigen-specific and broad adaptive immunity, and reduce immunosuppression within the TME, shaping a more effective environment for productive anti-tumor immunity (FIG. 3H).

### Example 27

### Microbial Anti-Tumor Vaccination in Melanoma

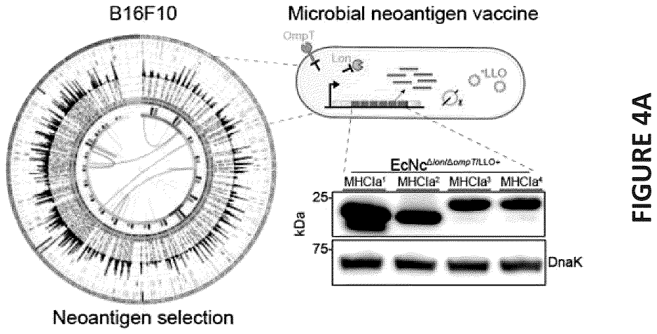

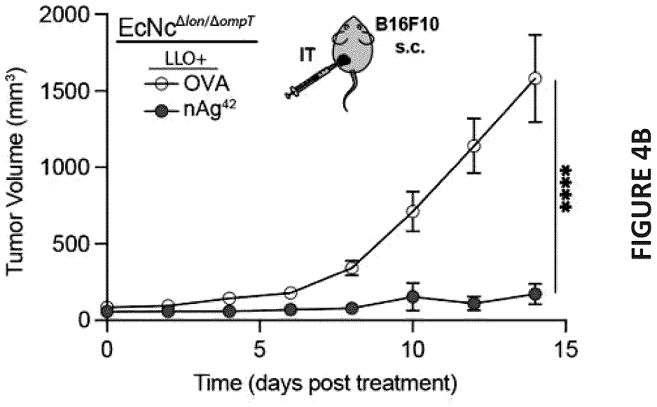

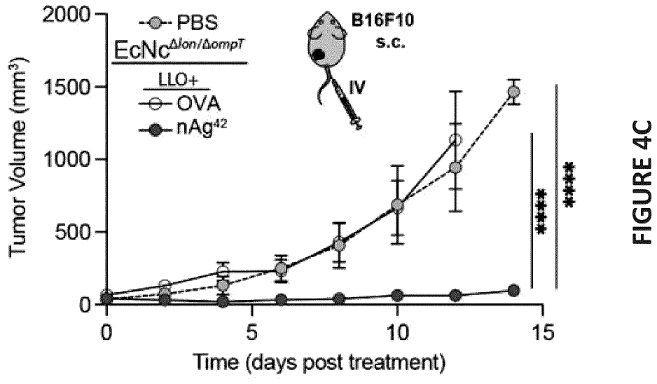

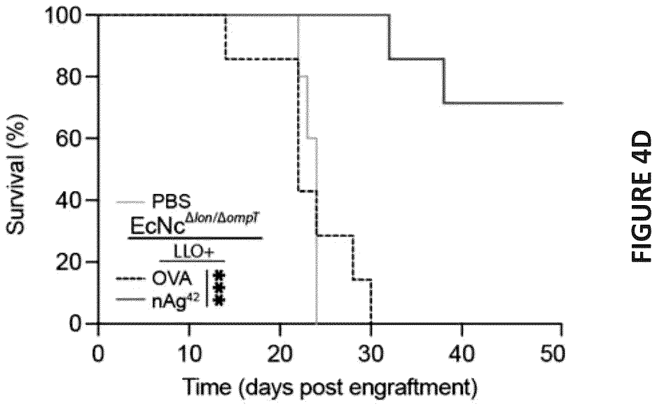

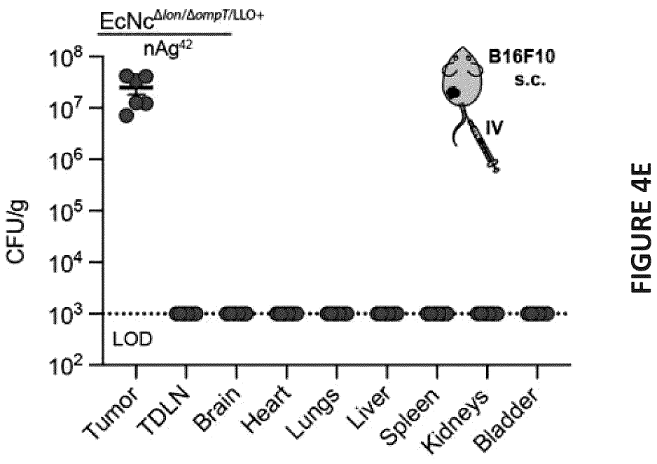

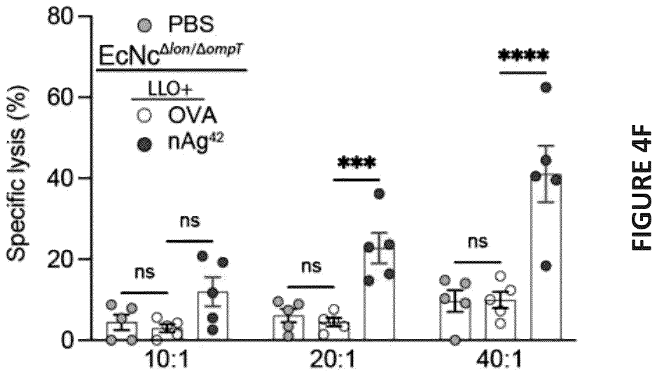

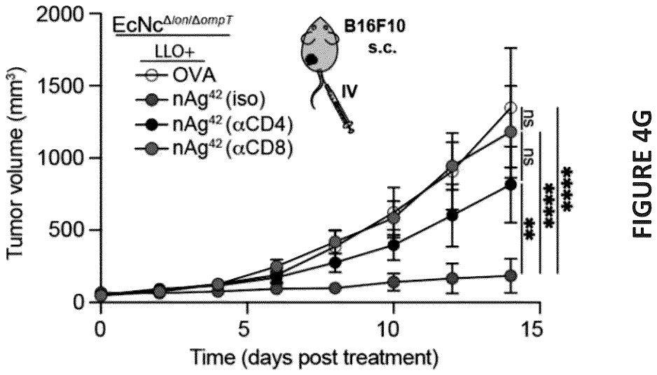

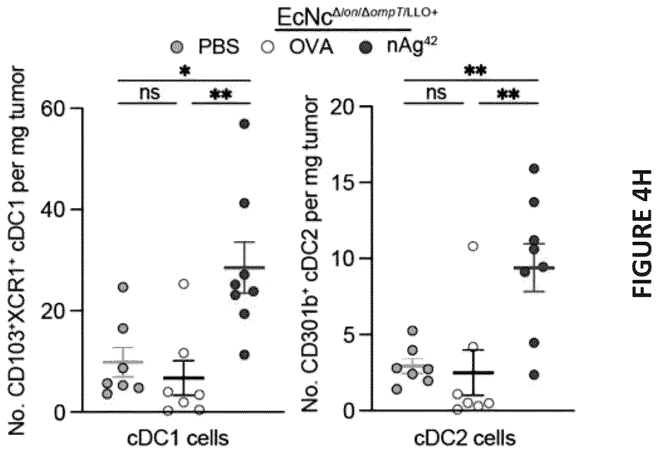

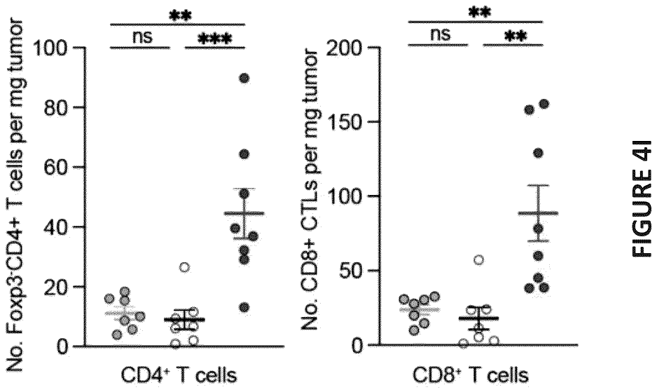

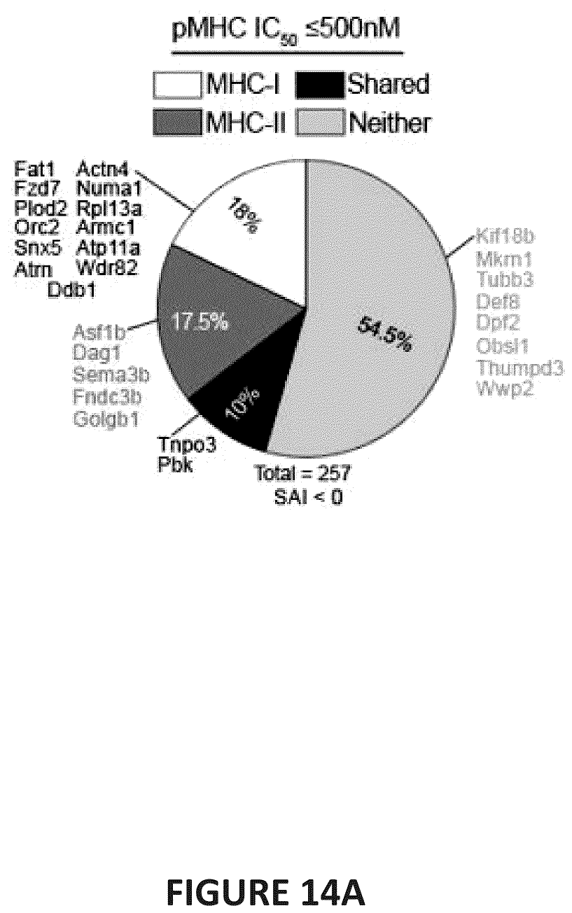

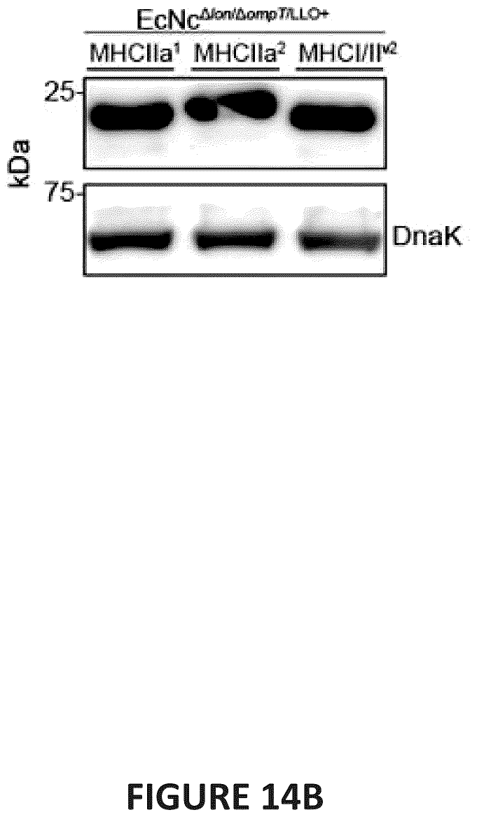

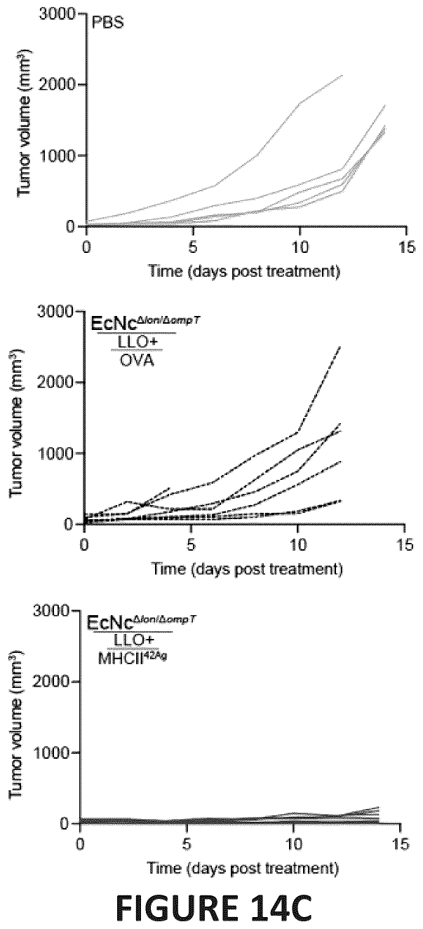

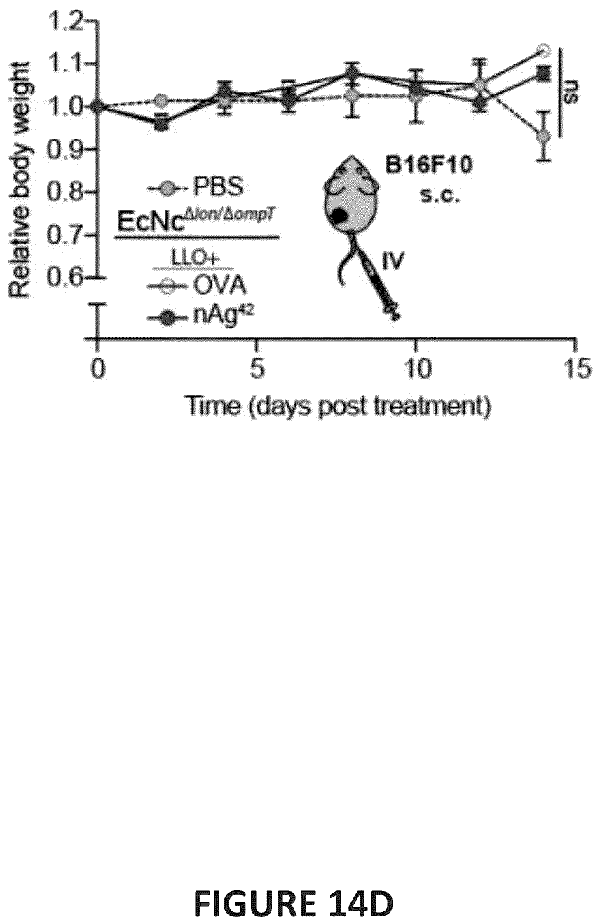

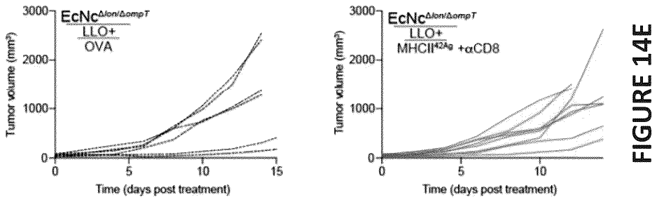

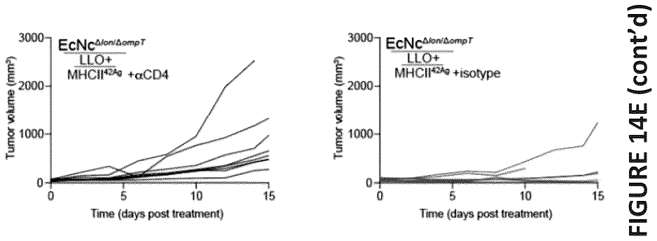

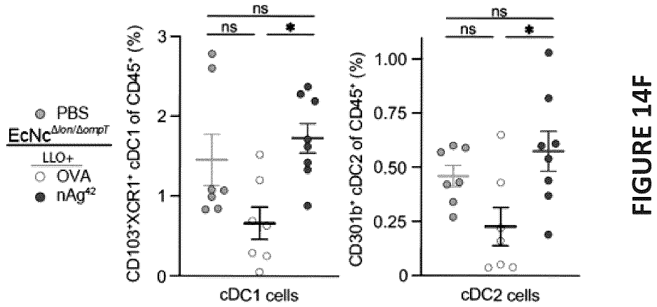

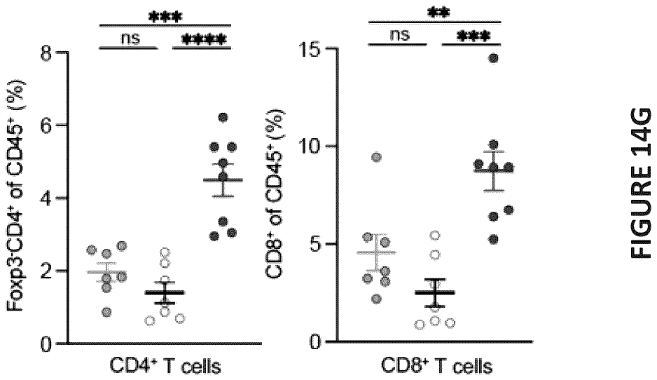

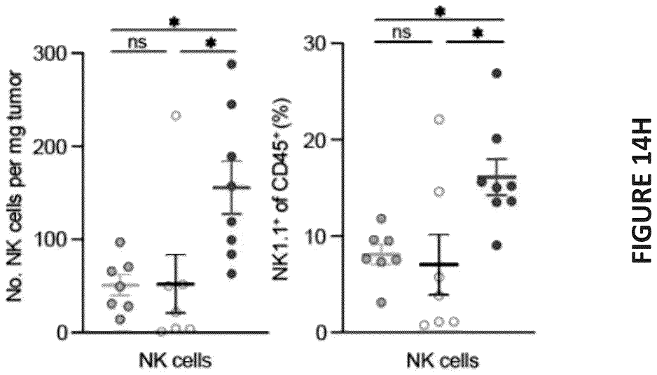

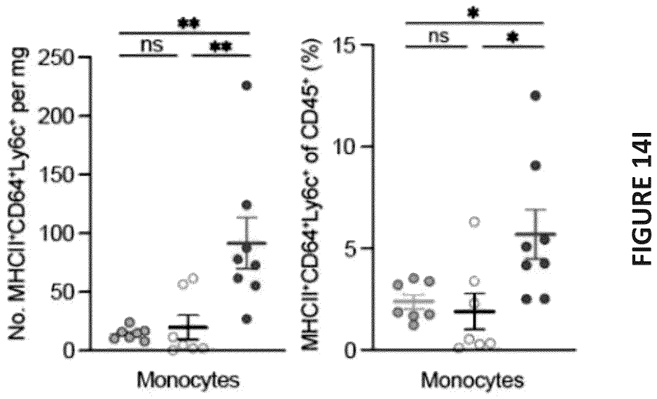

Neoantigens are generally unique to the individual patient, thus vaccination platforms must be able to flexibly incorporate and deliver diverse sets of neoantigens based on the unique mutations present in a particular tumor. To evaluate the suitability of the engineered microbial platform in this regard, paired exome and transcriptome sequencing was carried out on a second, more aggressive tumor cell type (B16F10 melanoma) grown orthotopically in C57BL/6 mice and designed tumor-specific therapeutics (FIG. 4A). An equivalent neoantigen prediction algorithm was developed and identified numerous putative B16F10-specific neoantigens (FIG. 14A). A set of seven constructs were devised from neoantigens of varying imputed MHC-I and MHC-II affinities, with each construct containing 6 unique predicted neoantigens (Table 3) and confirmed to be robustly expressed by EcNc^{ΔIon/ΔompT/LLO+} (FIGS. 4A and 14B).

TABLE 3
B16F10 mutant peptides in neoantigen constructs

| Construct | Gene | AA Mutation | Mutant Sequence in Construct | SEQ ID NO |
|---|---|---|---|---|
| MHCIa3 | Pcmtd1 | P222L | ESKNILAVSFAPLVQLSKNDNGTPDSVGLP | 73 |
| | Hipk3 | S702F | WQQVTPMAPAAATLTFEGMAGSQRLGDWGK | 74 |
| | Map1s | F881V | LPGGGAGHLDQNVFLRVRALCYVISGQGQR | 75 |
| | Haus6 | L176V | PQDMHKCLARSHVARNRFVQILQREHYVMQ | 76 |
| | Tab2 | S77T | QGFNVFGMPSTSGASNTTPHLGFHLGSKGT | 77 |
| | Sec23a | F94L | AKLWACNFCYQRNQLPPTYAGISELNQPAE | 78 |
| MHCIIa2 | Ipo13 | E441G | DTLMYVYEMLGAGLLSNLYDKLGRLLT | 79 |
| | Desi2 | N10S | GANQLWLSVYDMYWMNEYTSSIGIGV | 80 |
| | Rnf146 | A106V | TLLSPEELKAASRGNGEYVWYYEGRNG | 81 |
| | Ncor1 | H673P | KSEAQCKNFYFNYKRRPNLDNLLQQHK | 82 |
| | Man2b2 | D605N | RLVPVMNDCYILLFNQDTNMLHSIQDR | 83 |
| | Wrap53 | S144F | TYSFSQVPRYLSGFWSEFSTRSENFLK | 84 |
| MHCIIa3 | Vps13c | S1256P | IDLKAPVIVIPQSSLPTNAVVVDLGLIRVH | 85 |
| | Tmem246 | L11P | MTTSTSPAAMPLRRLRRLSWGSTAVQLFIL | 86 |
| | Dop1b | T838I | NHSQSLALVIEDKMKRYKISGNNPFFGKLQ | 87 |
| | Angel2 | D144N | RNVDSTCEDREDKFNFSVMSYNILSQDLLE | 88 |
| | Chd6 | D1602G | TAKHGLNRTDYYIMNGPQLSFLDAYRNYAQ | 89 |
| | Smarcc2 | G65A | LWVQLLQFQEEVFAKHVSNAPLTKLPIKCF | 90 |
| | Wipi2 | T304A | SYLPSQVTEMFNQGRAFAAVRLPFCGHKNI | 91 |
| MHCIIa1 | Sec16a | S970F | QTPQSPHPNAEKGPFEFVSSPAGNTSVMLV | 92 |
| | Ehbp1I1 | P1343A | EGGGGSGTYRVGNAQPSLADCLDAGDLAQR | 93 |
| | Atp6v1h | K146T | NRQDPFTVHMAARIIATLAAWGKELMEGSD | 94 |
| | Pnrc1 | T196I | INRQKSKYNLPLTKIISAKRNESDFWQDSA | 95 |
| | Klhl26 | E487A | YDPAADRWEPRAPMRAPRVLHAMLGAAGRI | 96 |
| | Cnst | G59A | AGDGPAGLTTSEGAMARATVSEQDSLNNNE | 97 |
| MHCIIa2 | Ints11 | D314N | KHIKAFDRTFANNPGPMVVFATPGMLHAGQ | 98 |
| | Hipk1 | E413G | YDQIRYISQTQGLPAGYLLSAGTKTTRFFN | 99 |
| | Atf7 | G249A | GPPVNNSGSISPSAHPMPSEAKMRLKATLH | 100 |
| | Gpatch4 | G192A | AQEQAFLAQLKGSKALATSQPLTDSEPSQK | 101 |
| | Heatr5a | T347A | AFLSHILSLVSQSNPKAAQTQIDAVCCRRC | 102 |
| | Fig4 | F498V | CVDCLDRTNTAQVMVGKCALAYQLYSL | 103 |
| MHCI/IIV1 | Etv5 | E472K | LFSMAFPDNQRPFLKAESKCPLNEEDTL | 104 |
| | Hccs | R199T | PSLVRFGGKAREYSPTARIRSWMGYEL | 105 |
| | Cubn | S3211L | LVKLTFNAFTLEEPSLPGKCTFDYVQI | 106 |
| | Dlg5 | P666S | PQPSKRVGSLTPPKSPRRSDSIKFQHR | 107 |
| | Dlg4 | R66Q | NDSILFVNEVDVQEVTHSAAVEALKEA | 108 |
| | Ift52 | D43A | LQKRLRSNWKIQSLKAEITSEKLIGVKLWI | 109 |
| MHCI/IIV2 | Mta1 | P547L | KPLEAVLRYLETHPRLPKPDPVKSSSSVLS | 110 |
| | Rpl12 | I82N | NRQAQIEVVPSASALINKALKEPPRDRKKQ | 111 |
| | Ctsd | G397S | WILGDVFIGSYYTVFDRDNNRVSFANAVVL | 112 |
| | Trrap | K2783R | QAQESYEKAMDKAKREHERSNASPAIFPEY | 113 |
| | Nsun2 | K72M | EELAWHTNLSRKILRMSPLLAKFHQFLVSE | 114 |
| | Pcmtd1 | P222L | ESKNILAVSFAPLVQLSKNDNGTPDSVGLP | 115 |

| | Mutant MHC-I | SEQ | Predicted MHC-I | MHC-I |
|---|---|---|---|---|
| Construct | Epitope | ID NO | IC₅₀ (nM) | Allele |
| MHCIa3 | VSFAPLVQL | 116 | 30.93 | H2Kb |
| | ATLTFEGM | 117 | 107.21 | H2Kb |
| | VFLRVRAL | 118 | 119.32 | H2Kb |
| | VARNRFVQI | 119 | 72.41 | H2Kb |
| | SGASNTTPHL | 120 | 188.87 | H2Db |
| | CNFCYQRNQL | 121 | 241.87 | H2Kb |
| | YVYEMLGAGL | 122 | 254.77 | H2Kb |
| MHCIIa2 | SVYDMYWM | 123 | 107.59 | H2Kb |
| | KAASRGNGEYV | 124 | 178.28 | H2Db |
| | FNYKRRPNL | 125 | 21.35 | H2Kb |
| | ILLFNQDTNM | 126 | 524.65 | H2Db |
| | QVPRYLSGF | 127 | 262.17 | H2Kb |
| MHCIIa3 | SSLPTNAVVV | 128 | 27.86 | H2Db |
| | TSPAAMPL | 129 | 123.91 | H2Kb |
| | ISGNNPFFGKL | 130 | 159.09 | H2Kb |
| | FNFSVMSYNIL | 131 | 452.07 | H2Kb |
| | YYIMNGPQL | 132 | 503.18 | H2Db |
| | FAKHVSNAPL | 133 | 538.04 | H2Db |
| | QGRAFAAV | 134 | 101.34 | H2Kb |
| MHCIIa1 | KGPFEFVSS | 135 | 4986.04 | H2Kb |
| | AQPSLADCL | 136 | 713.71 | H2Db |
| | MAARIIATL | 137 | 359.42 | H2Kb |
| | YNLPLTKII | 138 | 7072 | H2Kb |
| | RAPMRAPRV | 139 | 484.02 | H2Kb |
| | GAMARATV | 140 | 6796.53 | H2Kb |
| MHCIIa2 | RTFANNPGPM | 141 | 591.33 | H2Db |
| | AGYLLSAGT | 142 | 1011.08 | H2Kb |
| | ISPSAHPM | 143 | 272.28 | H2Kb |
| | KALATSQPL | 144 | 374.15 | H2Db |
| | AAQTQIDAV | 145 | 5195.03 | H2Db |
| | VMVGKCALA | 146 | 1185.97 | H2Kb |
| MHCI/IIv1 | KCPLNEEDTL | 147 | 2173.74 | H2Db |
| | KAREYSPT | 148 | 2378.58 | H2Kb |
| | NAFTLEEPSL | 149 | 3931.28 | H2Db |
| | KSPRRSDSI | 150 | 3310.37 | H2Kb |
| | VQEVTHSAAV | 151 | 5253.65 | H2Kb |
| | KIQSLKAEI | 152 | 5032.54 | H2Kb |
| MHCI/IIV2 | RYLETHPRL | 153 | 922.68 | H2Kb |
| | SALINKAL | 154 | 1058.79 | H2Db |
| | VSFANAWL | 155 | 14.98 | H2Db |
| | KAMDKAKRE | 156 | 19244.21 | H2Kb |
| | KILRMSPL | 157 | 39.75 | H2Kb |
| | VSFAPLVQL | 158 | 30.93 | H2Kb |

| | Mutant MHC-II | SEQ | Predicted MHC-II | MHC-II |
|---|---|---|---|---|
| Construct | Epitope | ID NO | IC₅₀ (nM) | Allele |
| MHCIa3 | KNILAVSFAPLVQLSK | 159 | 269.38 | H2-IAb |
| | AATLTFEGMAGSQRLG | 160 | 5250.81 | H2-IAb |
| | LDQNVFLRVRALCYVISGQ | 161 | 3944.2 | H2-IAb |
| | QDMHKCLARSHVARNRFVQ | 162 | 3080.52 | H2-IAb |
| | GFNVFGMPSTSGASNT | 163 | 73.1 | H2-IAb |
| | RNQLPPTYAGISELNQ | 164 | 1206.7 | H2-IAb |
| MHCIIa2 | VYEMLGAGLLSNLYDKLGR | 165 | 1170.5 | H2-IAb |
| | SVYDMYWMNEYTSSIGIGV | 166 | 1424.5 | H2-IAb |
| | TLLSPEELKAASRGNGEYV | 167 | 7840.8 | H2-IAb |
| | NFYFNYKRRPNLDNLL | 168 | 5770.7 | H2-IAb |
| | YILLFNQDTNMLHSIQ | 169 | 7182 | H2-IAb |
| | YLSGFWSEFSTRSENF | 170 | 2484.81 | H2-IAb |
| MHCIIa3 | VIVIPQSSLPTNAVVV | 171 | 231.5 | H2-IAb |
| | MTTSTSPAAMPLRRLR | 172 | 92.5 | H2-IAb |
| | MKRYKISGNNPFFGKL | 173 | 663.4 | H2-IAb |
| | REDKFNFSVMSYNILS | 174 | 654.97 | H2-IAb |
| | TDYYIMNGPQLSFLDA | 175 | 151 | H2-IAb |
| | EEVFAKHVSNAPLTKL | 176 | 230.3 | H2-IAb |
| | SQVTEMFNQGRAFAAVRLP | 177 | 59.7 | H2-IAb |
| MHCIIa1 | AEKGPFEFVSSPAGNTSVM | 178 | 74.8 | H2-IAb |
| | GGSGTYRVGNAQPSLADCL | 179 | 80.1 | H2-IAb |
| | RQDPFTVHMAARIIATLAA | 180 | 190.2 | H2-IAb |
| | KSKYNLPLTKIISAK | 181 | 460.17 | H2-IAb |
| | EPRAPMRAPRVLHAML | 182 | 99.2 | H2-IAb |
| | DGPAGLTTSEGAMARATVS | 183 | 198.3 | H2-IAb |
| MHCIIa2 | TFANNPGPMVVFATPGMLH | 184 | 54.2 | H2-IAb |
| | TQGLPAGYLLSAGTKTTRF | 185 | 384.6 | H2-IAb |
| | VNNSGSISPSAHPMPSEAK | 186 | 111 | H2-IAb |
| | FLAQLKGSKALATSQP | 187 | 113 | H2-IAb |
| | SHILSLVSQSNPKAAQTQI | 188 | 203.4 | H2-IAb |
| | DCLDRTNTAQVMVGKCALA | 189 | 2981.7 | H2-IAb |
| MHCI/IIV1 | LFSMAFPDNQRPFLKAESK | 190 | 1149.5 | H2-IAb |
| | LVRFGGKAREYSPTARIRS | 191 | 297.1 | H2-IAb |
| | VKLTFNAFTLEEPSLP | 192 | 738.5 | H2-IAb |
| | PSKRVGSLTPPKSPRR | 193 | 1027.3 | H2-IAb |
| | VDVQEVTHSAAVEALK | 194 | 323.73 | H2-IAb |
| | RLRSNWKIQSLKAEITSEK | 195 | 2910.8 | H2-IAb |
| MHCI/IIv2 | AVLRYLETHPRLPKPDPVK | 196 | 544.4 | H2-IAb |
| | QIEVVPSASALINKAL | 197 | 216.2 | H2-IAb |
| | FDRDNNRVSFANAVVL | 198 | 232.9 | H2-IAb |
| | KAKREHERSNASPAIF | 199 | 1245.3 | H2-IAb |
| | LSRKILRMSPLLAKFH | 200 | 674.7 | H2-IAb |
| | KNILAVSFAPLVQLSK | 201 | 269.38 | H2-IAb |

The anti-tumor efficacy of these therapeutics was then tested against advanced B16F10 tumors. When established orthotopic tumors were injected with microbial therapeutics intratumorally, tumors grew progressively after treatment with EcNc^{ΔIon/ΔompT/LLO+} expressing the strong irrelevant xenoantigen ovalbumin (OVA), whereas treatment with the equal-parts combination of all seven construct expressing strains—encompassing 42 unique B16F10 neoantigens (nAg⁴²)—significantly repressed growth in EcNc^{ΔIon/ΔompT/LLO+} nAg⁴² treated tumors over the same time course (FIG. 4B). Similarly, intravenous treatment with EcNc^{ΔIon/ΔompT/LLO+} nAg⁴² potently restrained orthotopic tumor growth, with 72% of nAg⁴² treated mice alive 50 days post-tumor engraftment, while all control group mice succumbed to malignancy by day 24 or 30 (FIGS. 4C, 4D and 14C). Treatment with intravenous microbial vaccines again induced no significant body weight change as compared to PBS treated mice (FIG. 14D).

To evaluate the tissue biodistribution of the microbial neoantigen vaccines after systemic administration in this setting, organs were examined after intravenous injection of EcNc^{ΔIon/ΔompT/LLO+} nAg⁴². As observed for BALB/c mice with CT26 tumors, live microbial vectors specifically colonized the B16F10 tumor at high density without detectable presence in any other organs examined (FIG. 4E). To confirm that the microbial B16F10 neoantigen vaccine generated T cells capable of direct tumor cell killing, tumor-free C57BL/6 mice were intravenously administered microbial therapeutics and purified splenic T cells were co-incubated with B16F10 tumor cells in vitro. Indeed, T cells from mice treated intravenously with EcNc^{ΔIon/ΔompT/LLO+} nAg⁴² but not EcNc^{ΔIon/ΔompT/LLO+} OVA demonstrated enhanced killing of B16F10 tumor cells (FIG. 4F). These data verify tumor-specific colonization and antigen-specific T cell induction by microbial neoantigen vaccines in B16F10 melanoma. To assess the dependency of anti-tumor efficacy on CD4⁺ and CD8⁺ T cells in vivo, we depleted either CD4⁺ or CD8⁺ T cells from mice treated intravenously with EcNc^{ΔIon/ΔompT/LLO+} nAg⁴². Depletion of either CD4⁺ or CD8⁺ T cells ablated therapeutic efficacy, indicating that both conventional CD4⁺ and cytotoxic CD8⁺ T cells are required for productive anti-tumor immunity in vivo (FIGS. 4G, 14E).

### Example 28

### Microbial Vectors Modulate Anti-Tumor Immunity and Metastases in Melanoma

To characterize the immunologic changes associated with anti-tumor efficacy in this alternate model, immunophenotyping of the orthotopic B16F10 tumors was performed 8 days after intravenous microbial treatment. Tumors treated intravenously with EcNc^{ΔIon/ΔompT/LLO+} nAg⁴² as compared to controls had significantly higher numbers and frequencies of cDC1 and cDC2 cells, conventional CD4⁺ and cytotoxic CD8⁺ infiltrating T cells, NK cells, and inflammatory monocytes (FIGS. 4H, 4I, and 14F-14I).

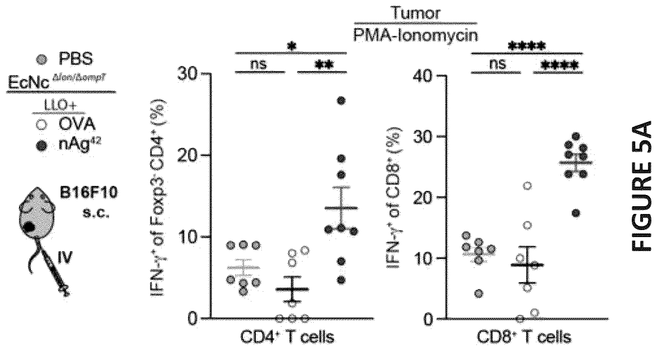

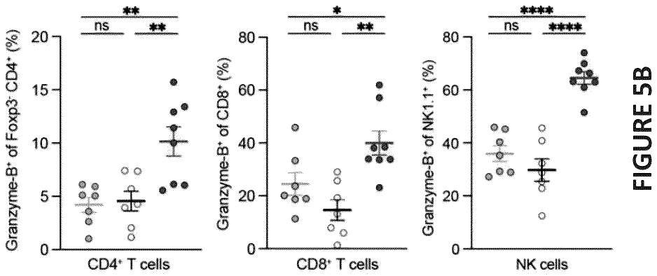

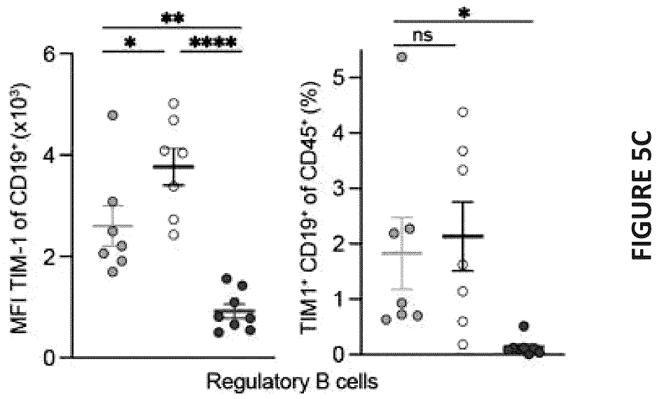

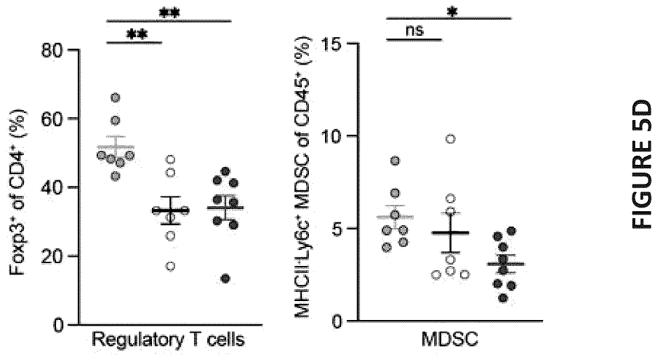

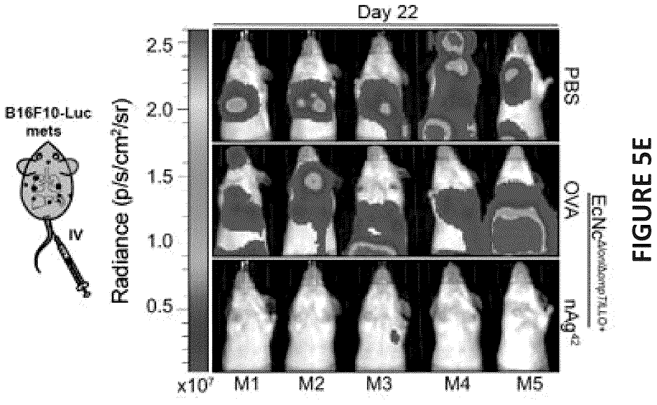

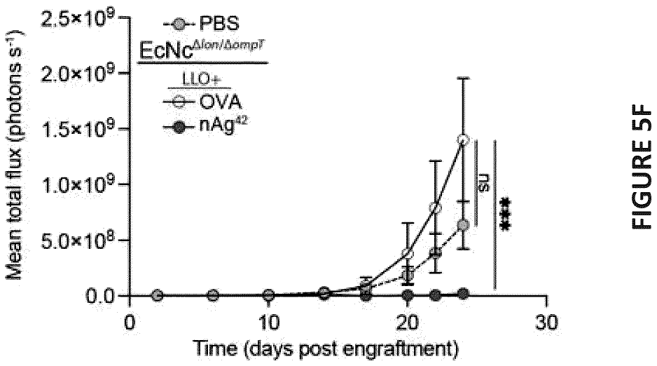

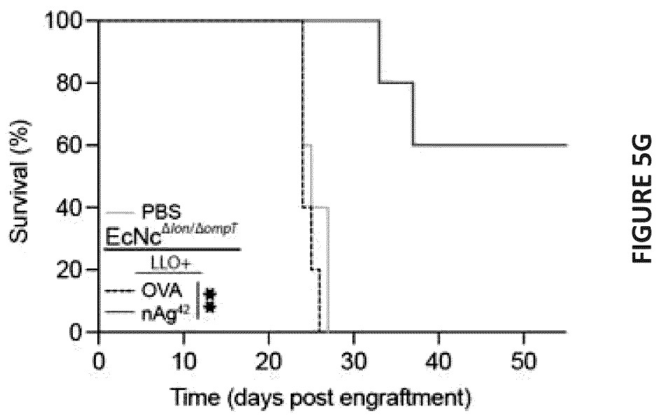

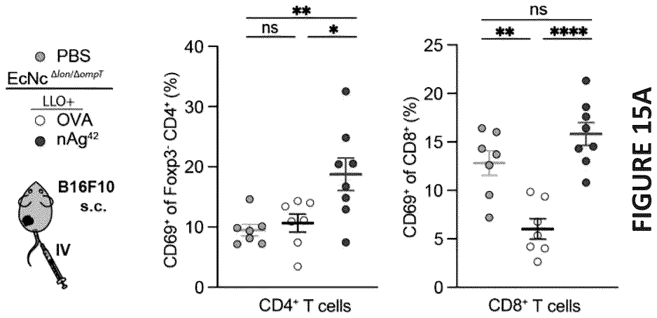

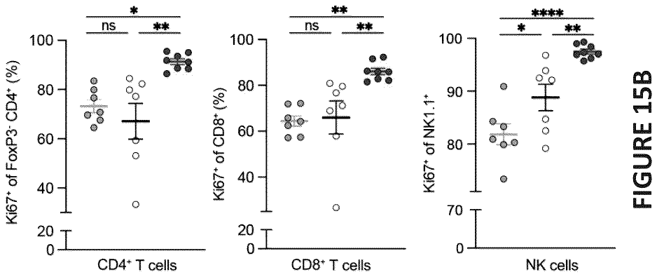

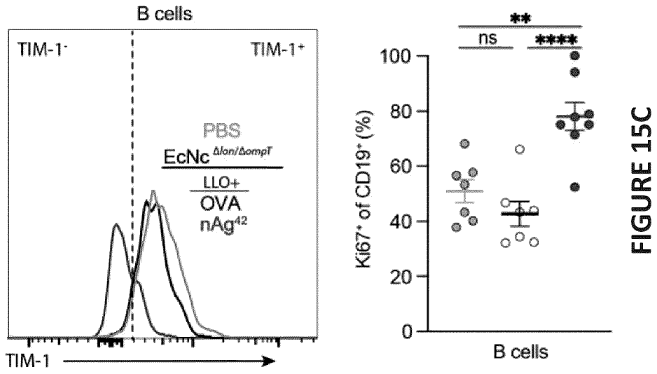

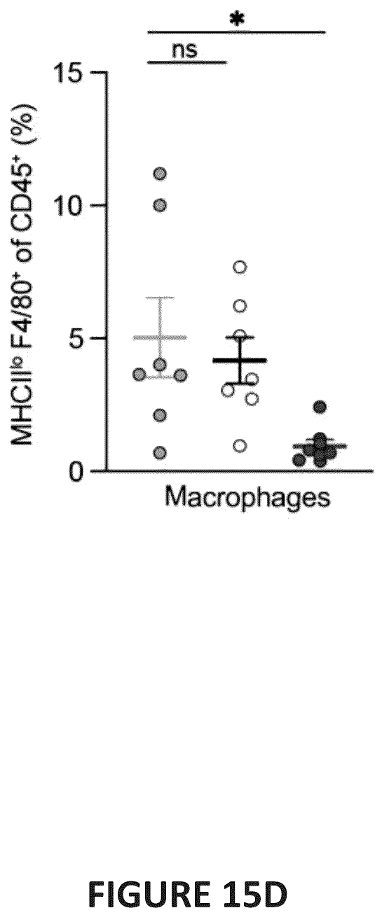

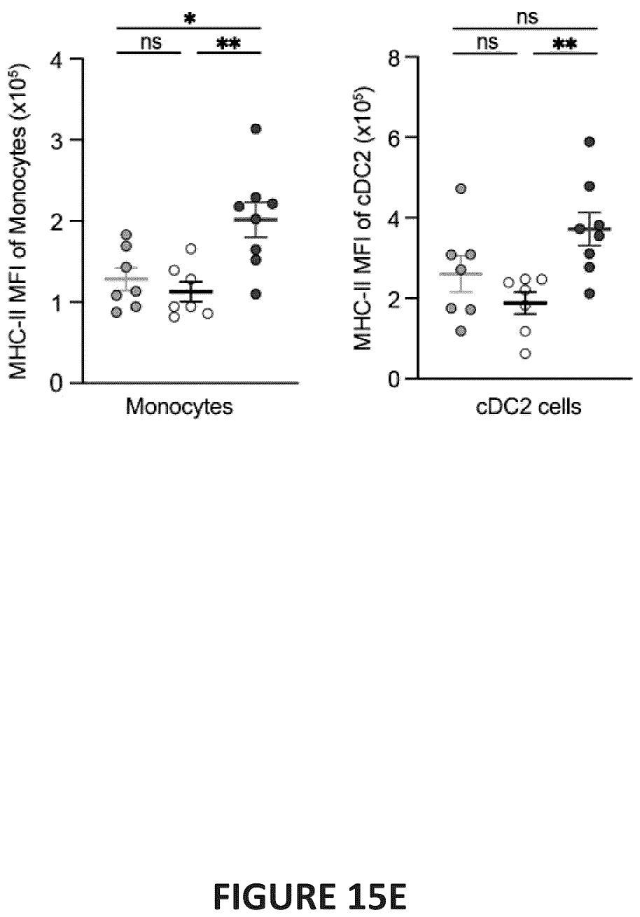

Analyses of the intratumoral lymphoid compartment revealed enhanced expression of CD69 on Foxp3⁻CD4⁺ and CD8⁺ TILs, and significantly increased frequencies of IFN-γ secreting conventional Foxp3⁻CD4⁺ and cytotoxic CD8⁺ TILs after restimulation with PMA and ionomycin in EcNc^{ΔIon/ΔompT/LLO+} nAg⁴²-treated tumors, indicating enhanced T cell activation and effector cytokine production within the TME (FIGS. 5A and 15A). Tumor-infiltrating Foxp3⁻CD4⁺ and CD8⁺ T cells and NK cells also expressed significantly higher levels of granzyme-B after EcNc^{ΔIon/ΔompT/LLO+} nAg⁴² treatment, suggestive of amplified cytolytic function (FIG. 5B). Consistent with enduring activity of anti-tumor immunity, higher levels of proliferating tumor-infiltrating CD4⁺ and CD8⁺ T cells, and NK cells as assessed by Ki-67 staining were also observed (FIG. 15B).

In addition to the enhanced activation of tumor-infiltrating T and NK cells, treatment with EcNc^{ΔIon/ΔompT/LLO+} nAg⁴² significantly reduced TIM-1 expression by tumor-infiltrating CD19⁺ B cells and thus the frequency of regulatory TIM-1⁺ B cells—an important immunosuppressive cell population in the B16F10 model—and increased B cell proliferation (FIGS. 5C and 15C). Moreover, EcNc^{ΔIon/ΔompT/LLO+} nAg⁴² vaccination reduced the frequency of immunosuppressive Foxp3⁺ regulatory T cells, myeloid-derived suppressor cells (MDSC), and MHC-II^{lo} macrophages within tumors (FIGS. 5D and 15D). Infiltrating monocytes and dendritic cells in EcNc^{ΔIon/ΔompT/LLO+} nAg⁴²-treated tumors exhibited increased expression of MHC-II (FIG. 15E), suggestive of enhanced antigen presentation capacity of these cell types within tumors. Overall, these data demonstrate that intravenous microbial tumor neoantigen vaccination mediates immunologic restructuring within the melanoma TME, recruiting APCs and activating NK cells, and CD4⁺ and CD8⁺ T cells while diminishing central immunosuppressive cell populations.

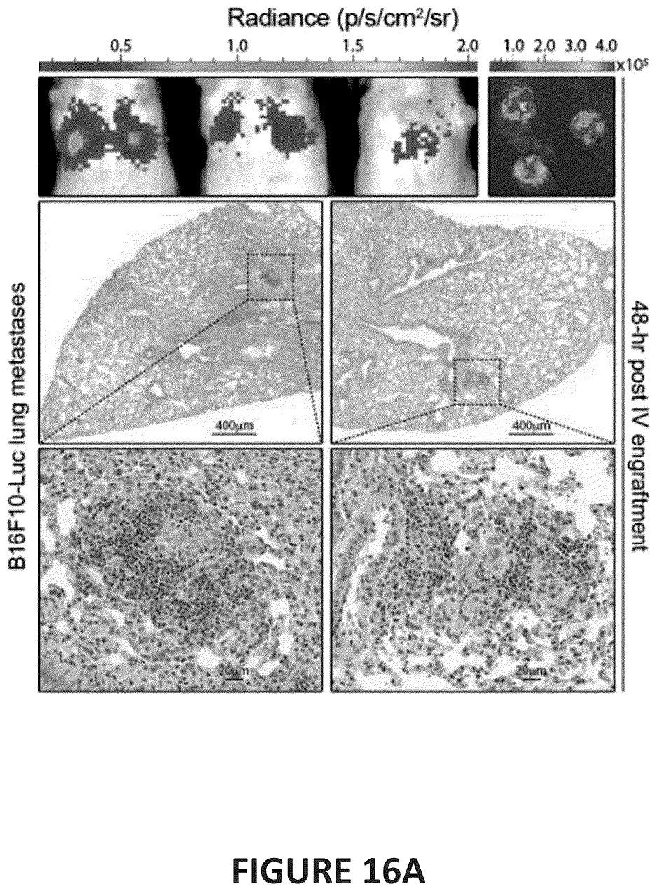

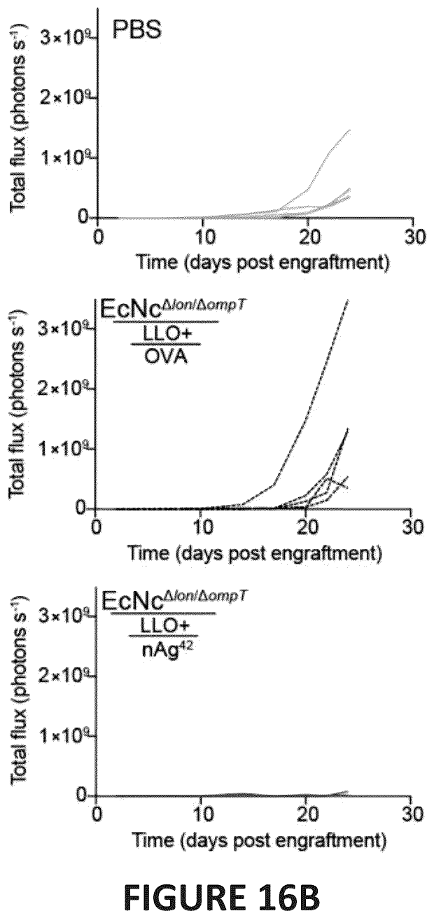

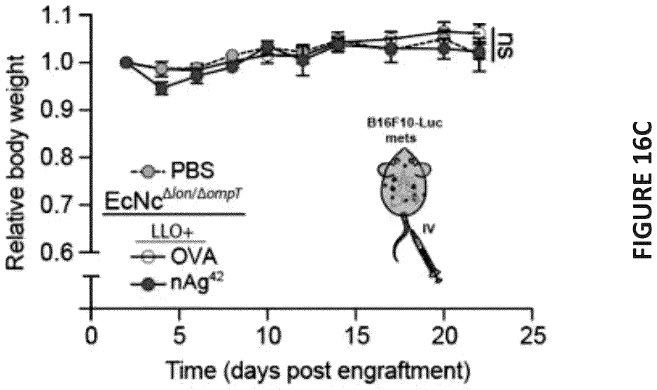

Given the robust anti-tumor efficacy induced by vaccination in orthotopic B16F10, the efficacy of EcNc^{ΔIon/ΔompT/LLO+} nAg⁴² in established, systemic B16F10-Luc metastases was investigated (FIG. 16A). Whereas systemic metastases rapidly progressed in PBS or EcNc^{ΔIon/ΔompT/LLO+} OVA treated mice, EcNc^{ΔIon/ΔompT/LLO+} nAg⁴² strongly inhibited metastatic growth (FIGS. 5E, 5F and 16B). Treatment with EcNc^{ΔIon/ΔompT/LLO+} nAg⁴² significantly extended survival, with 60% of mice surviving to 55 days, with no metastases detectable by imaging, whereas all control treated mice had died by day 27 (FIG. 5G). Treatment was well-tolerated, with no significant weight change relative to PBS (FIG. 16C). These data demonstrate that the microbial tumor neoantigen vaccination system stimulates robust and effective anti-tumor immunity in vivo after intravenous administration in established, systemic metastatic melanoma.

While this invention has been disclosed with reference to particular embodiments, it is apparent that other embodiments and variations of the inventions disclosed herein can be devised by others skilled in the art without departing from the true spirit and scope thereof. The appended claims include all such embodiments and equivalent variations.

What is claimed is:

1. An engineered Escherichia coli Nissle bacteria comprising one or more heterologous nucleic acids that encode one or more neoantigen epitopes present on the surface of a tumor cell.

2. The engineered Escherichia coli Nissle bacteria of claim 1, which comprises between one and ten therapeutic vectors each comprising between one and ten heterologous nucleic acids that each encode a neoantigen epitope.

3. The engineered Escherichia coli Nissle bacteria of claim 1, comprising two or more heterologous nucleic acids, wherein the two or more heterologous nucleic acids are separated by a nucleic acid sequence encoding a spacer that is between three and seven amino acids in length.

4. The engineered Escherichia coli Nissle bacteria of claim 1, wherein the spacer has the amino acid sequence GGGGS (SEQ ID NO: 202).

5. The engineered Escherichia coli Nissle bacteria of claim 1, which lack cryptid plasmids pMUT1 and pMUT2.

6. The engineered Escherichia coli Nissle bacteria of claim 1, which lack a protease selected from the group of ion, OmpT, or both.

7. The engineered Escherichia coli Nissle bacteria of claim 1, which constitutively expresses cytosolic listeriolysin.

8. A method of fabricating a personalized vaccine for a subject with cancer, comprising: (a) optionally obtaining a biological sample from the subject, wherein the biological sample comprises one or more nucleic acids derived from a cancer cell present in the subject; (b) identifying one or more potential neoantigens from the one or more nucleic acids by comparing nucleic acid sequences of the one or more nucleic acids to nucleic acid sequences from a non-cancerous cell of the same type; (c) optionally screening peptides comprising the one or more potential neoantigens for an immunogenic response; (d) optionally identifying one or more epitopes on one or more neoantigens that produced an immunogenic response; and (e) transforming a Escherichia coli Nissle bacteria with one or more vectors comprising one or more heterologous nucleic acids that encode one or more neoantigen epitopes, producing the personalized vaccine thereby.

9. A method of treating a subject with cancer with a personalized vaccine, comprising: (a) optionally obtaining a biological sample from the subject, wherein the biological sample comprises one or more nucleic acids derived from a cancer cell present in the subject; (b) identifying one or more potential neoantigens from the one or more nucleic acids by comparing nucleic acid sequences of the one or more nucleic acids to nucleic acid sequences in a non-cancerous cell of the same type; (c) optionally screening peptides comprising the one or more potential neoantigens for an immunogenic response; (d) optionally identifying one or more epitopes on one or more neoantigens that produced an immunogenic response; (e) transforming a Escherichia coli Nissle bacteria with one or more vectors comprising one or more heterologous nucleic acids that encode one or more neoantigen epitopes, producing a personalized vaccine thereby; and (f) administering the personalized vaccine to the subject.

10. The method of claim 9, further comprising administering a second personalized vaccine produced by (g) obtaining a second biological sample from the subject after the subject exhibits an immune response, wherein the second biological sample comprises one or more tumor-infiltrating lymphocytes (TILs); (h) screening the one or more TILs with one or more peptides comprising the epitopes on one or more neoantigens that produced an immunogenic response; (i) selecting one or more neoantigens, wherein the selected neoantigens are different from the neoantigens in the personalized vaccine; (j) transforming a second Escherichia coli Nissle bacteria with one or more vectors comprising one or more heterologous nucleic acids that encode one or more neoantigen epitopes present on the selected neoantigens; and (k) administering the second personalized vaccine to the subject.

11. The method of claim 9, wherein the personalized vaccine is administered to the subject intravenously or intratumorally.

12. The method of claim 9, wherein the one or more neoantigen epitopes have an amino acid sequence set forth as any one of SEQ ID NOs: 1-201.

13. The method of claim 10, wherein the one or more neoantigen epitopes have an amino acid sequence set forth as any one of SEQ ID NOs: 1-201.

14. A pharmaceutical composition comprising the engineered Escherichia coli Nissle bacteria of claim 1 and one or more pharmaceutically acceptable carriers, diluents, or excipients.

15. The pharmaceutical composition of claim 12, comprising a plurality of different engineered Escherichia coli Nissle bacteria strains, wherein each strain expresses a different combination of neoantigen epitopes.
